SEQUENCE LISTING (12 sequences)

SEQ ID NO: 1 - DNA, 59 bp, Artificial Sequence
  Synthetic Primer
tcgttaactaattaatttaagaaggagatatacatatggtatcaaaaggggaagagttg

SEQ ID NO: 2 - DNA, 40 bp, Artificial Sequence
  Synthetic Primer
ttagaataggcgcgccttatttgtacagttcgtccatgcc

SEQ ID NO: 3 - DNA, 62 bp, Artificial Sequence
  Synthetic Primer
tcgttaactaattaatttaagaaggagatatacatatgagtaaaggagaagaacttttcact

SEQ ID NO: 4 - DNA, 46 bp, Artificial Sequence
  Synthetic Primer
ttagaataggcgcgccttattatttgtatagttcatccatgccatg

SEQ ID NO: 5 - DNA, 59 bp, Artificial Sequence
  Synthetic Primer
tcgttaactaattaatttaagaaggagatatacatatggtgagtgtgattaaaccagag

SEQ ID NO: 6 - DNA, 42 bp, Artificial Sequence
  Synthetic Primer
ttagaataggcgcgccttaggaatgagctactgcatcttcta

SEQ ID NO: 7 - DNA, 39 bp, Artificial Sequence
  Synthetic Primer
tataatagcatgcacataacaccaaaaagaagaaggtgc

SEQ ID NO: 8 - DNA, 36 bp, Artificial Sequence
  Synthetic Primer
ccgcaaaggcgcctgtcactttgcttgatatatgag

SEQ ID NO: 9 - DNA, 60 bp, Artificial Sequence
  Synthetic Primer
aatacgccgttaactgactttattatcttattatatttttttaacgtttctcaccgatgc

SEQ ID NO: 10 - DNA, 60 bp, Artificial Sequence
  Synthetic Primer
ggaggggctcacgaccatacttacatgtcaacgataatacaaaatataatacaaaatata

SEQ ID NO: 11 - DNA, 60 bp, Artificial Sequence
  Synthetic Primer
ttgaatacgccgttaactgactttattatcttattatatttttttaacgtttctcaccga

SEQ ID NO: 12 - DNA, 23 bp, Artificial Sequence
  Synthetic Primer
ggaggggctcacgaccatactta

(19) United States
(12) Patent Application Publication
(10) Pub. No.: US 2018/0125066 A1
(43) Pub. Date: May 10, 2018

(54) SURFACES COMPRISING ATTACHED QUORUM SENSING MODULATORS

(71) Applicant: The Trustees of Princeton University, Princeton, NJ (US)

(72) Inventors: Bonnie L. Bassler, Princeton, NJ (US); Howard A. Stone, Princeton, NJ (US); Min Young Kim, Seoul (KR); Thomas William Muir, Princeton, NJ (US); Aishan Zhao, Princeton, NJ (US)

(73) Assignee: The Trustees of Princeton University, Princeton, NJ (US)

(21) Appl. No.: 15/793,090

(22) Filed: Oct. 25, 2017

Related U.S. Application Data
(63) Continuation-in-part of application No. PCT/US16/29213, filed on Apr. 25, 2016.
(60) Provisional application No. 62/478,485, filed on Mar. 29, 2017.
(60) Provisional application No. 62/459,482, filed on Feb. 15, 2017.
(60) Provisional application No. 62/152,896, filed on Apr. 26, 2015.

(51) Int. Cl. A01N 25/10; A01N 25/08; A01N 43/16; A01N 35/04; C12Q 1/02

(52) U.S. Cl. CPC A01N 25/10; A01N 25/08; C12Q 1/025; A01N 35/04; A01N 43/16

(57) ABSTRACT

The invention relates to compositions comprising QS modulating molecules attached to a surface via a linker. This QS modulator attached surface can then be used to modulate QS, biofilm production, biofilm streamer production and/or virulence factor production. The length of the linker that attaches the QS modulating molecule to the surface as well as the surface coverage density are features that impact QS modulation on surfaces. These QS modulator attached surfaces can be used to treat areas known to contain human pathogens notorious for causing hospital-acquired infections as well as fatal infections that occur outside of health care settings. Other surfaces that can be coated according to embodiments of the invention include abiotic materials, such as intravenous catheters, implants, medical devices, and cooling towers. Preferred microorganisms that can be treated with the compositions of the invention include, but are not limited to S. aureus and/or P. aeruginosa. The QS modulator attached surface also demonstrates exceptional stability.

### CROSS-REFERENCE TO RELATED APPLICATIONS

This application is a Continuation in Part of PCT/US16/29213 filed on Apr. 25, 2016, which claims priority to U.S. 62/152,896 filed on Apr. 26, 2015, and this application also claims priority to U.S. 62/459,482 filed on Feb. 15, 2017 and U.S. 62/478,485 filed on Mar. 29, 2017, all which are incorporated by reference in their entirety.

### STATEMENT REGARDING FEDERALLY SPONSORED RESEARCH OR DEVELOPMENT

This invention was made with government support under Grant No MCB-1119232, Grant No. MCB-1344191 and Grant No. MCB-0948112 awarded by the National Science Foundation and Grant No. GM-065859 awarded by the National Institutes of Health. The government has certain rights in the invention.

### SEQUENCE LISTING

The instant application contains a Sequence Listing which has been submitted in ASCII format via EFS-Web and is hereby incorporated by reference in its entirety. Said ASCII copy, created on Jan. 15, 2018, is named PRN-06-CIP_Sequence_Listing_ST25.txt and is 2,746 bytes in size.

### BACKGROUND

In a process referred to as quorum sensing, microorganisms, such as bacteria, communicate using chemical signaling molecules called autoinducers. Quorum sensing involves the production, release, and population-wide detection of extracellular signal molecules called autoinducers¹⁻⁸. By monitoring increases and decreases in autoinducer concentration, quorum-sensing bacteria track changes in cell-population density and synchronously switch into and out of collective group behaviors. Quorum sensing allows bacteria to collectively carry out tasks that would be unsuccessful if carried out by an individual bacterium acting alone.

Both Gram-positive and Gram-negative infectious bacteria, which include human, animal, plant, and marine pathogens, use quorum sensing to control virulence. Quorum sensing also controls biofilm and in some cases streamer formation. Biofilms are communities of bacterial cells adhered to surfaces and encased in a self-produced matrix of extracellular polymeric substances. In most environments, bacteria are found predominantly in biofilms. These biofilms are also widespread in industrial systems and are associated with increased risk of infection when found in clinical environments and in indwelling medical devices. These bacterial biofilm communities can cause chronic infections in humans by colonizing, for example, in medical implants, heart valves, or lungs.

In settings involving fluid flow across the biofilm, as in the environment for example in rivers or in industrial and medical systems that are subject to flow, filamentous biofilms, called streamers, can be formed. These streamers can have a dramatic effect on the biofilm environment. In rivers, for example, the biofilm streamers can increase transient storage and cycling of nutrients and can enhance the retention of suspended particles. In industrial and medical settings, the biofilm streamers have been associated with increased issues associated with clogging and pressure drops.

Bacterial infections are treated with bactericidal or bacteriostatic molecules that impede at least five major processes: cell wall formation, DNA replication, transcription, translation or tetrahydrofolic acid synthesis. Existing methods for treating bacterial infection unfortunately exacerbate the growing antibiotic resistance problem because they inherently select for growth of bacteria that can resist the drug.

For example, Staphylococcus aureus is a human pathogen notorious for causing hospital-acquired infections as well as fatal infections that occur outside of health care settings. S. aureus is responsible for multiple fatal diseases including bacteremia, toxic shock syndrome, and medical device-related infections. Many strains of S. aureus are multi-drug resistant (i.e. Methicillin-resistant Staphylococcus aureus (MRSA))^{24,25}. In this context, the quorum-sensing system plays a central regulatory role in S. aureus pathogenicity and biofilm dynamics^{1,8}.

S. aureus infections that are associated with abiotic materials, such as intravenous catheters and implants, are of primary concern as S. aureus readily colonizes such medical devices, forming biofilms, biofilm streamers and initiates virulence factor production under these conditions.

In fact, methicillin-resistant S. aureus (MRSA) is a major concern due to its potent virulence coupled with resistance to many antibiotics. MRSA is the most widespread cause of hospital-associated infections in the United States and Europe with a high mortality rate. S. aureus and MRSA cause a variety of infections ranging from minor skin infections to serious illnesses such as infections of indwelling medical devices, osteomyelitis, endocarditis, sepsis, and toxic shock syndrome. S. aureus is just one example of a microorganism that uses quorum-sensing-mediated communication to control virulence factor production and to regulate biofilm formation.

Administration of compounds in general can be difficult, due to lack of or inefficient compound transport to the site of infection, compound insolubility, compound instability, complex topography (i.e. grooves/crevices at the site of infection), and resistance to chemical perturbations¹⁸⁻²¹. These issues complicate therapies, often necessitating delivery of large and frequent doses of the compounds, which in turn, can cause complications such as cytoxicity or off target effects.

Thus, what is needed are methods of modulating quorum sensing to combat bacterial infections which can be effectively delivered without causing adverse side effects. Preferably, these methods will demonstrate improved efficacy and safety over conventional methods.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

The present invention targets quorum sensing (“QS”) systems utilized by microorganisms, which are crucial in microorganism infection and pathogenicity. This technology involves conjugation of QS modulating molecules (either QS antagonists or agonists¹⁴⁻¹⁷) to a surface to influence and/or control QS-controlled phenotypes, wherein the attached molecule retains the ability to influence bacterial behaviors.

More specifically, presented herein are methods of attaching compounds to a surface to interact with receptors involved in QS, and the ability of the modified surface to resist degradation, e.g., from long-term storage, repeated infections, host plasma components and/or flow-generated stresses. The techniques presented herein can be used to make colonization-resistant materials against S. aureus and other pathogens and promote beneficial behaviors of bacteria on surfaces. By tethering quorum modulating molecules to surfaces, site-specific targeting (e.g., at the site of the medical device) can be achieved for a wide range of applications to combat bacterial infections.

Preferred examples of altered QS phenotypes (also referred to as traits) include, but are not limited to, significant reductions in biofilm formation, biofilm streamer formation, and virulence factor production. This technology can be immediately applied to many current and urgent issues in healthcare settings, such as the accidental introduction of pathogens into patients during medical procedures and the entry of bacteria at wound sites. This technology can be used to modify existing (e.g., medical, food processing, agricultural, etc.) devices, create new devices, and can also be applied as a direct treatment of bacterial infections for patients. Beyond medicine, this technology can also be applied to other fields including, but not limited to, industrial and engineering processes, food processing, cooling towers, and mining. Additional applications include prevention of biofilm formation to reduce biofouling in industry, to halt recurrent infections in medicine, and to suppress virulence factor production to limit pathogen infectivity⁹⁻¹¹.

Additionally, the techniques presented herein may be used to enhance QS-controlled behaviors in beneficial bacteria. For example, biofilm formation from beneficial bacterial is used in wastewater treatment and food processing, and, in the context of the microbiome, such beneficial/commensal bacterial are thought to fend off invading pathogens by relying on QS-controlled traits^{12,13}.

Thus, the present invention relates to a method of modulating QS, biofilm production, biofilm streamer production, and/or virulence factor production by a microorganism using: (1) an antagonist to decrease QS, or (2) an agonist to increase QS, wherein the QS modulator is attached to a surface through a linker. A microorganism that is exposed to the surface will exhibit altered biofilm production, biofilm streamer production, and/or virulence factor production. In some embodiments, inhibiting QS will lead to a decrease in biofilm production, biofilm streamer production, and/or virulence factor production. In other embodiments, agonizing QS will lead to a decrease in biofilm production, biofilm streamer production, and/or virulence factor production.

For example, a QS modulator molecule attached to a surface can be used to promote or inhibit the pathogenic behaviors of the microorganism on a surface, including, but not limited to, a medical device and/or at any wound site in patients. By conjugating a QS modulating molecule to a surface, the surface will then promote or inhibit QS, in turn, leading to an alteration in biofilm formation, biofilm streamer formation, and/or virulence factor production.

Alternatively, a QS modulator molecule attached to a surface can be used to promote beneficial behaviors of the microorganism on a surface, including, but not limited to, in food processing, engineering or industrial settings. By conjugating a QS modulating molecule to a surface, the surface will then control QS regulated beneficial phenotypes, including, but not limited to, enzyme or metabolite production, such as enzymes that can degrade plastics and petroleum products, enzymes that help digestion in humans, and metabolites that can be consumed by animals or humans.

In specific microorganisms, a QS agonist can actually repress biofilm formation and/or virulence factor expression. These microorganisms are virulent at low cell density and in response to QS autoinducers, can escape the host cell defenses. For example, Vibrio cholerae dissociates from the host's epithelial cells at high cell densities to become extremely contagious. In this situation, a QS agonist attached to a surface, rather than a QS antagonist, could be used to inhibit biofilm formation and thus repress virulence. Examples of such microorganisms include, but are not limited to S. aureus, Vibrio cholerae, Vibrio parahaemolyticus, Vibrio vulnificus, and Vibrio harveyi.

In other specific microorganisms, a QS antagonist can repress biofilm formation and/or virulence factor expression. These microorganisms are virulent at high cell density, and in response to QS autoinducers, can damage the host cells. In this situation, a QS antagonist attached to a surface, rather than a QS agonist, could be used to inhibit biofilm formation and/or repress virulence. Examples of such microorganisms include, but are not limited to Pseudomonas aeruginosa and Enterococcus faecalis.

As used herein, the QS autoinducers, a biofilm, a biofilm streamer, and/or a virulence factor are produced or formed by a microorganism(s). In preferred embodiments, the microorganism is selected from the following groups: bacteria, archaea, protozoa, fungi, and/or algae. In further embodiments, the bacteria, archaea, protozoa, fungi, and/or algae are pathogenic to humans, animals and/or plants. Alternatively, the bacteria, archaea, protozoa, fungi, and/or algae are beneficial to humans, animals and/or plants. In further embodiments the bacteria, archaea, protozoa, fungi, or algae are common to industrial settings, including, but not limited to, industrial fluid handling processes, medical processes, agricultural processes, and/or machinery. In further embodiments, the bacteria, archaea, protozoa, fungi, or algae are common to an apparatus and/or process that involves fluid flow.

In still further embodiments, the bacteria are selected from the following genera: Abiotrophia, Achromobacter, Acidaminococcus, Acidovorax, Acinetobacter, Actinobacillus, Actinobaculum, Actinomadura, Actinomyces, Aerococcus, Aeromonas, Afipia, Agrobacterium, Alcaligenes, Alloiococcus, Alteromonas, Amycolata, Amycolatopsis, Anabaena, Anabaenopsis, Anaerobospirillum, Anaerorhabdus, Aphanizomenon, Arachnia, Arcanobacterium, Arcobacter, Arthrobacter, Atopobium, Aureobacterium, Bacillus, Bacteroides, Balneatrix, Bartonella, Bergeyella, Bifidobacterium, Bilophila, Bordetella, Borrelia, Brachyspira, Branhamella, Brevibacillus, Brevibacterium, Brevundimonas, Brucella, Burkholderia, Buttiauxella, Butyrivibrio, Calymmatobacterium, Camesiphon, Campylobacter, Capnocytophaga, Capnylophaga, Cardiobacterium, Catonella, Cedecea, Cellulomonas, Centipeda, Chlamydia, Chlamydophila, Chromobacterium, Chryseomonas, Chyseobacterium, Citrobacter, Clostridium, Collinsella, Comamonas, Corynebacterium, Coxiella, Cryptobacterium, Cyanobacteria, Cylindrospermopsis, Delftia, Dermabacter, Dermatophilus, Desulfomonas, Desulfovibrio, Dialister, Dichelobacter, Dolosicoccus, Dolosigranulum, Edwardsiella, Eggerthella, Ehrlichia, Eikenella, Empedobacter, Enterobacter, Enterococcus, Erwinia, Erysipelothrix, Escherichia, Eubacterium, Ewingella, Exiguobacterium, Facklamia, Filifactor, Flavimonas, Flavobacterium, Francisella, Fusobacterium, Gardnerella, Gemella, Globicatella, Gloeobacter, Gordona, Haemophilus, Hafnia, Hapalosiphon, Helicobacter, Helococcus, Hemophilus, Holdemania, Ignavigranum, Johnsonella, Kingella, Klebsiella, Kocuria, Koserella, Kurthia, Kytococcus, Lactobacillus, Lactococcus, Lautropia, Leclercia, Legionella, Leminorella, Leptospira, Leptospirae, Leptotrichia, Leuconostoc, Listeria, Listonella, Lyngbya, Megasphaera, Methylobacterium, Microbacterium, Micrococcus, Microcystis, Mitsuokella, Mobiluncus, Moellerella, Moraxella, Morganella, Mycobacterium, Mycoplasma, Myroides, Neisseria, Nocardia, Nocardiopsis, Nodularia, Nostoc, Ochrobactrum, Oeskovia, Oligella, Orientia, Paenibacillus, Pantoea, Parachlamydia, Pasteurella, Pediococcus, Peptococcus, Peptostreptococcus, Phormidium, Photobacterium, Photorhabdus, Phyllobacterium, Phytoplasma, Planktothrix, Plesiomonas, Porphyromonas, Prevotella, Propionibacterium, Proteus, Providencia, Pseudoanabaena, Pseudomonas, Pseudonocardia, Pseudoramibacter, Psychrobacter, Rahnella, Ralstonia, Rhodococcus, Rickettsia, Rochalimaea, Roseomonas, Rothia, Ruminococcus, Salmonella, Schizothrix, Selenomonas, Serpulina, Serratia, Shewenella, Shigella, Simkania, Slackia, Sphaerotilus, Sphingobacterium, Sphingomonas, Spirillum, Spiroplasma, Spirulina, Staphylococcus, Stenotrophomonas, Stomatococcus, Streptobacillus, Streptococcus, Streptomyces, Succinivibrio, Sutterella, Suttonella, Tatumella, Tissierella, Trabulsiella, Treponema, Trichodesmium, Tropheryma, Tsakamurella, Turicella, Umezakia, Ureaplasma, Vagococcus, Veillonella, Vibrio, Weeksella, Wolinella, Xanthomonas, Xenorhabdus, Yersinia, and Yokenella.

In still further embodiments the bacteria are selected from the following species: Acinetobacter baumannii, Actinobacillus actinomycetemcomitans, Actinobacillus pleuropneumoniae, Actinomyces bovis, Actinomyces Israelii, Bacillus anthracis, Bacillus ceretus, Bacillus coagulans, Bacillus liquefaciens, Bacillus popillae, Bacillus subtilis, Bacillus thuringiensis, Bacteroides distasonis, Bacteroides fragilis, Bacteroides thetaiotaomicron, Bacteroides vulgatus, Bartonella bacilliformis, Bartonella Quintana, Beneckea parahaemolytica, Bordetella bronchiseptica, Bordetella parapertussis, Bordetella pertussis, Borelia burgdorferi, Brevibacterium lactofermentum, Brucella abortus, Brucella canis, Brucella melitensis, Brucella suis, Burkholderia cepacia, Burkholderia mallei, Burkholderia pseudomallei, Campylobacter fetus, Campylobacter jejuni, Campylobacter pylori, Cardiobacterium hominis, Chlamydia pneumoniae, Chlamydia psittaci, Chlamydia trachomatis, Chlamydophila abortus, Chlamydophila caviae, Chlamydophila felis, Chlamydophila pneumonia, Chlamydophila psittaci, Chryseobacterium eningosepticum, Clostridium botulinum, Clostridium butyricum, Clostridium coccoides, Clostridium dijficile, Clostridium leptum, Clostridium tetani, Corynebacterium xerosis, Cowdria ruminantium, Coxiella burnetii, Edwardsiella tarda, Ehrlichia sennetsu, Eikenella corrodens, Elizabethkingia meningoseptica, Enterobacter aerogenes, Enterobacter cloacae, Enterococcus faecalis, Escherichia coli, Escherichia hirae, Flavobacterium meningosepticum, Fluoribacter bozemanae, Francisella tularensis, Francisella tularensis biovar Tularensis, Francisella tularensis subsp. Holarctica, Francisella tularensis subsp. nearctica, Francisella tularensis subsp. Tularensis, Francisella tularensis var. palaearctica, Fudobascterium nucleatum, Fusobacterium necrophorum, Haemophilus ducreyi, Haemophilus influenzae, Helicobacter pylori, Kingella kingae, Klebsiella mobilis, Klebsiella oxytoca, Klebsiella pneumoniae, Lactobacillus acidophilus, Lactobacillus casei, Lactobacillus hilgardii, Lactobacillus pentosus, Lactobacillus plantarum, Lactobacillus rhamnosus, Lactococcus lactis, Legionella bozemanae corrig., Legionella pneumophila, Leptospira alexanderi, Leptospira borgpetersenii, Leptospira fainei, Leptospira inadai, Leptospira interrogans, Leptospira kirschneri, Leptospira noguchii, Leptospira santarosai, Leptospira weilii, Leuconostoc lactis, Leuconostoc oenos, Listeria ivanovii, Listeria monocytogenes, Moraxella catarrhalis, Morganella morganii, Mycobacterium africanum, Mycobacterium avium, Mycobacterium avium subspecies paratuberculosis, Mycobacterium bovis, Mycobacterium bovis strain BCG, Mycobacterium intracellulare, Mycobacterium kansasii, Mycobacterium leprae, Mycobacterium marinum, Mycobacterium tuberculosis, Mycobacterium typhimurium, Mycobacterium ulcerans, Mycoplasma hominis, Mycoplasma mycoides, Mycoplasma pneumoniae, Neisseria gonorrhoeae, Neisseria meningitidis, Neorickettsia sennetsu, Nocardia asteroides, Orientia tsutsugamushi, Pasteurella haemolytica, Pasteurella multocida, Plesiomonas shigelloides, Propionibacterium acnes, Proteus mirabilis, Proteus morganii, Proteus penneri, Proteus rettgeri, Proteus vulgaris, Providencia alcalifaciens, Providencia rettgeri, Pseudomonas aeruginosa, Pseudomonas mallei, Pseudomonas pseudomallei, Pyrococcus abyssi, Rickettsia akari, Rickettsia canadensis, Rickettsia canadensis corrig, Rickettsia conorii, Rickettsia montanensis, Rickettsia montanensis corrig, Rickettsia prowazekii, Rickettsia rickettsii, Rickettsia sennetsu, Rickettsia tsutsugamushi, Rickettsia typhi, Rochalimaea quintana, Salmonella arizonae, Salmonella choleraesuis subsp. arizonae, Salmonella enterica subsp. Arizonae, Salmonella enteritidis, Salmonella paratyphi, Salmonella typhi, Salmonella typhimurium, Selenomonas nominantium, Selenomonas ruminatium, Serratia marcescens, Shigella boydii, Shigella dysenteriae, Shigella flexneri, Shigella sonnei, Spirillum minus, Staphylococcus aureus, Staphylococcus epidermidis, Staphylococcus equi, Staphylococcus lugdunensis, Stenotrophomonas maltophila, Streptobacillus moniliformis, Streptococcus agalactiae, Streptococcus bovis, Streptococcus ferus, Streptococcus pneumoniae, Streptococcus pyogenes, Streptococcus viridans, Streptomyces ghanaenis, Streptomyces hygroscopicus, Streptomyces phaechromogenes, Treponema carateum, Treponema denticola, Treponema pallidum, Treponema pertenue, Vibrio cholerae, Vibrio parahaemolyticus, Vibrio vulnificus, Xanthomonas maltophilia, Yersinia enterocolitica, Yersinia pestis, Yersinia pseudotuberculosis, and Zymomonas mobilis.

In still further embodiments, the bacteria are from the class of bacteria known as Fusospirochetes. In further embodiments, the microorganism comprises fungi. In still further embodiments, the fungi are selected from the following genera: Candida, Saccharomyces, and Cryptococcus.

Such pathogenic bacteria can cause bacterial infections and disorders related to such infections that include, but are not limited to, the following: acne, rosacea, skin infection, pneumonia, otitis media, sinusitus, bronchitis, tonsillitis, and mastoiditis related to infection by Streptococcus pneumoniae, Haemophilus influenzae, Moraxella catarrhalis, Staphylococcus aureus, Peptostreptococcus spp. or Pseudomonas spp.; pharynigitis, rheumatic fever, and glomerulonephritis related to infection by Streptococcus pyogenes, Groups C and G streptococci, Clostridium diptheriae, or Actinobacillus haemolyticum; respiratory tract infections related to infection by Mycoplasma pneumoniae, Legionella pneumophila, Streptococcus pneumoniae, Haemophilus influenzae, or Chlamydia pneumoniae; uncomplicated skin and soft tissue infections, abscesses and osteomyelitis, and puerperal fever related to infection by Staphylococcus aureus, coagulase-positive staphylococci (i.e., S. epidermidis, S. hemolyticus, etc.), S. pyogenes, S. agalactiae, Streptococcal groups C-F (minute-colony streptococci), viridans streptococci, Corynebacterium spp., Clostridium spp., or Bartonella henselae; uncomplicated acute urinary tract infections related to infection by S. saprophyticus or Enterococcus spp.; urethritis and cervicitis; sexually transmitted diseases related to infection by Chlamydia trachomatis, Haemophilus ducreyi, Treponema pallidum, Ureaplasma urealyticum, or Nesseria gonorrheae; toxin diseases related to infection by S. aureus (food poisoning and Toxic shock syndrome), or Groups A, S, and C streptococci; ulcers related to infection by Helicobacter pylori; systemic febrile syndromes related to infection by Borrelia recurrentis; Lyme disease related to infection by Borrelia burgdorferi; conjunctivitis, keratitis, and dacrocystitis related to infection by C. trachomatis, N gonorrhoeae, S. aureus, S. pneumoniae, S. pyogenes, H influenzae, or Listeria spp.; disseminated Mycobacterium avium complex (MAC) disease related to infection by Mycobacterium avium, or Mycobacterium intracellulare; gastroenteritis related to infection by Campylobacter jejuni; odontogenic infection related to infection by viridans streptococci; persistent cough related to infection by Bordetella pertussis; gas gangrene related to infection by Clostridium perfringens or Bacteroides spp.; skin infection by S. aureus, Propionibacterium acne; atherosclerosis related to infection by Helicobacter pylori or Chlamydia pneumoniae; or the like. The QS modulating molecule attached to surfaces as described herein can be used to treat any of these disorders.

In certain embodiments the disease or disorder that can be treated with QS modulating molecule attached surfaces as described herein include sepsis, pneumonia, lung infections from cystic fibrosis, otitis media, chronic obstructive pulmonary disease, a urinary tract infection, and/or combinations thereof. In other embodiments, the QS modulating molecule attached surfaces described herein can be used to reduce and/or eliminate a medical device-related infection. In further embodiments, the QS modulating molecule attached surfaces described herein can be used to treat a periodontal disease, such as gingivitis, periodontitis or breath malodor. In still further embodiments, the QS modulating molecule attached surfaces described herein can be used to treat infections, including but not limited to those infections caused by bacteria. In some embodiments, the bacteria are Gram-negative or Gram-positive bacteria. Non-limiting examples of diseases and/or disorders that can be treated and/or prevented with the QS modulating molecule attached surfaces include otitis media, prostatitis, cystitis, bronchiectasis, bacterial endocarditis, osteomyelitis, dental caries, periodontal disease, infectious kidney stones, acne, Legionnaire's disease, chronic obstructive pulmonary disease (COPD), and cystic fibrosis.

In one specific example, subjects with cystic fibrosis can display with an accumulation of biofilm in the lungs and digestive tract. Subjects afflicted with COPD, such as emphysema and chronic bronchitis, display a characteristic inflammation of the airways wherein airflow through such airways, and subsequently out of the lungs, is chronically obstructed. Infections, including biofilm-related disorders, also encompass infections on implanted/inserted devices, medical device-related infections, such as infections from biliary stents, orthopedic implant infections, and catheter-related infections (e.g., kidney, vascular, peritoneal, etc.). An infection can also originate from sites where the integrity of the skin and/or soft tissue has been compromised. Non-limiting examples include dermatitis, ulcers from peripheral vascular disease, burn injury, and trauma. All of these diseases and/or disorders can be treated using the QS modulating molecule attached surfaces as described herein.

A QS modulating molecule (e.g., an antagonist) attached surface as described herein can be used to inhibit QS, thereby inhibiting biofilm formation, biofilm streamer formation and/or virulence factor expression in the healthcare field, in waste water treatment facilities or to treat those microorganisms that up-regulate these traits in response to QS autoinducers. A QS modulating molecule (e.g., an agonist) attached surface as described herein can be used to promote QS thereby inhibiting biofilm formation, biofilm streamer formation and/or virulence factor expression in the healthcare field, in waste water treatment facilities or to treat those microorganisms that down-regulate these traits in response to QS autoinducers. Either of these types of QS modulating molecules could be used to alter QS-controlled traits in beneficial bacteria.

In a preferred embodiment, the QS modulator surface described herein attaches the QS modulating molecule to a surface through a chemical bond including, but not limited to, a covalent bond. Additionally, the QS modulator attached surfaces can be placed in a static environment or under pressure, such as in a fluid flow environment or under controlled pressure. The surface can be any material, e.g., glass, metals, including, but not limited to, stainless steel metals, silicon, plastic, polymers, metals, and/or ceramic materials.

In preferred embodiments, a surface can comprise a polymer, including, but not limited to, polyethylene, polypropylene, polystyrene, polyester, polyester PLA and other biosorbable plastics, polycarbonate, polyvinyl chloride, polyethersulfone, polyacrylate (e.g., Acrylic, PMMA), hydrogel (e.g., acrylate), polysulfone, polyetheretherketone, thermoplastic elastomers (e.g., TPE, TPU), thermoset elastomers, silicone, poly-p-xylylene (e.g., Parylene), fluoropolymers, or any combination thereof.

In other preferred embodiments, a surface can comprise a metal, including, but not limited to stainless steel, cobalt-base alloys, titanium, titanium-base alloys, and/or shape memory alloy.

In other preferred embodiments, a surface can comprise a ceramic material including, but not limited to, glass ceramics, calcium phosphate ceramics, and/or carbon-based ceramics. Moreover, the surface can have any shape such as, e.g., small particles, including but not limited to nanoparticles, and/or flat and/or curved surfaces as described herein.

More specifically, the surface can be circular, oval, square, rectangular, flat and/or irregularly shaped. In further embodiments, the surface may have a constant cross-sectional area and/or it may be variable (e.g., it may constrict in certain areas and/or expand in others). In other embodiments, the surface may change shape along its length. In still further embodiments, the surface may comprise depressions, gutter, groove and/or furrow. This depression may be shallow, deep, narrow and/or wide. In still further embodiments, the surface may be part of a larger device or machine. In other embodiments, the surface may be part of an implantable medical device. In still further embodiments, the surface may be part of machinery used in industrial processes. In some embodiments, the surface may be very small (i.e. just large enough for fluid and bacterial cells to flow through). In some embodiments, the surface may be very large (i.e. the large culverts and pools used in a waste water treatment facility.) In still further embodiments, the surface may be circular. In still further embodiments, the surface may be part of a pipe, a cooling tower, a medical device, and/or other industrial fluid handling machinery.

In some embodiments, the surface comprises at least one biofilm streamer promotion element. In further embodiments, the surface has at least 2, 3, 4, 5, 6, 7, 8, 9, 10, 11, 12, 13, 14, 15, 16, 17, 18, 19, 20, 25, 30, 35, 40, 45, 50, 60, 70, 80, 90 and/or 100 or more biofilm streamer promotion elements. In further embodiments, the surface has between 20-100 biofilm streamer promotion elements.

In further embodiments, the surface is a curved channel, a channel with at least one turn, a channel with at least one corner, an edge projecting into the lumen of the channel, a mound projecting into the lumen of the channel, a channel with roughened surfaces, and/or one or more objects placed on the surface. In further embodiments, the surface has at least 2, 3, 4, 5, 6, 7, 8, 9, 10, 11, 12, 13, 14, 15, 16, 17, 18, 19, 20, 25, 30, 35, 40, 45, 50, 60, 70, 80, 90 and/or 100 or more turns. In further embodiments, the surface has between 20-100 turns. In further embodiments, the surface has at least 2, 3, 4, 5, 6, 7, 8, 9, 10, 11, 12, 13, 14, 15, 16, 17, 18, 19, 20, 25, 30, 35, 40, 45, 50, 60, 70, 80, 90 and/or 100 or more edges projecting onto the surface. In further embodiments, the surface has between 20-100 edges projecting onto the surface. In further embodiments, the surface has at least 2, 3, 4, 5, 6, 7, 8, 9, 10, 11, 12, 13, 14, 15, 16, 17, 18, 19, 20, 25, 30, 35, 40, 45, 50, 60, 70, 80, 90 and/or 100 or more mounds projecting onto the surface. In further embodiments, the surface has between 20-100 mounds projecting onto the surface. In further embodiments, the surface has at least 2, 3, 4, 5, 6, 7, 8, 9, 10, 11, 12, 13, 14, 15, 16, 17, 18, 19, 20, 25, 30, 35, 40, 45, 50, 60, 70, 80, 90 and/or 100 or more roughened surfaces. In further embodiments, the surface has between 20-100 roughened surfaces. In further embodiments, the surface has at least 2, 3, 4, 5, 6, 7, 8, 9, 10, 11, 12, 13, 14, 15, 16, 17, 18, 19, 20, 25, 30, 35, 40, 45, 50, 60, 70, 80, 90 and/or 100 or more objects placed on the surface. In further embodiments, the surface has between 20-100 objects placed on the surface.

In further embodiments, the surface has at least 2 corners, at least 3 corners, at least 4 corners, at least 5 corners, at least 6 corners, at least 7 corners, at least 8 corners, at least 9 corners, at least 10 corners, at least 11 corners, at least 12 corners, at least 13 corners, at least 14 corners, at least 15 corners, at least 16 corners, at least 17 corners, at least 18 corners, at least 19 corners, at least 20 corners, at least 25 corners, at least 30 corners, at least 35 corners, and/or at least 40 corners or more. In further embodiments, the surface has between 20-40 corners. In still further embodiments, the surface has 1 turn about every 100 μm, every 200 μm, every 300 μm, every 400 μm, every 500 μm, every 600 μm, every 700 μm, every 800 μm, 900 μm, and/or every 1000 μm. Other dimensions could also be used.

Additionally, embodiments include a combination of any of these elements (e.g., turns, corners, edges, mounds, roughened surfaces, and/or objects) included on the surface.

In still further embodiments, the surface has NAFION® granules placed on the surface. In further embodiments, the surface comprises glass beads. In further embodiments, the surface comprises sand particles. In further embodiments, the surface comprises a welded polypropylene feed spacer mesh. In further embodiments, the surface is found on a stent. In still further embodiments, the surface is a bare-metal stent.

In some embodiments, the surface is subject to a laminar flow. In further embodiments, the flow of the fluid is characterized by a Reynolds number of less than 2000, of less than 1500, of less than 1000, of less than 750, of less than 500, of less than 400, of less than 300, of less than 200, of less than 100, of less than 50, of less than 25, of less than 10, of less than 5, of less than 4, of less than 3, of less than 2, and/or of less than 1. Other dimensions could also be used.

In some embodiments, the surface is subject to a turbulent flow. In further embodiments, the flow of the fluid is characterized by a Reynolds number of greater than 2000.

In some embodiments, the surface is subject to a shear stress. In further embodiments, the shear stress is characterized by a number between 0.01 and 100 Pa, between 0.01 and 90 Pa, between 0.01 and 80 Pa, between 0.01 and 70 Pa, between 0.01 and 60 Pa, between 0.01 and 50 Pa, between 0.01 and 40 Pa, between 0.01 and 30 Pa, between 0.01 and 20 Pa, between 0.01 and 10 Pa, between 0.02 and 10 Pa, between 0.03 and 10 Pa, between 0.04 and 10 Pa, between 0.05 and 10 Pa, between 0.06 and 10 Pa, between 0.07 and 10 Pa, between 0.08 and 10 Pa, between 0.09 and 10 Pa, between 0.1 and 10 Pa, between 0.02 and 100 Pa, between 0.03 and 100 Pa, between 0.04 and 100 Pa, between 0.05 and 100 Pa, between 0.06 and 100 Pa, between 0.07 and 100 Pa, between 0.08 and 100 Pa, between 0.09 and 100 Pa, between 0.1 and 100 Pa, between 0.1 and 90 Pa, between 0.1 and 80 Pa, between 0.1 and 70 Pa, between 0.1 and 60 Pa, between 0.1 and 50 Pa, between 0.1 and 40 Pa, between 0.1 and 30 Pa, between 0.1 and 20 Pa, between 0.02 and 90 Pa, between 0.03 and 80 Pa, between 0.04 and 70 Pa, between 0.05 and 60 Pa, between 0.06 and 50 Pa, between 0.07 and 40 Pa, between 0.08 and 30 Pa, and/or between 0.09 and 20 Pa. Other dimensions could also be used.

In the present invention, any linker can be used to attach the QS modulator to the surface. Examples of linkers are well known in the art, and can be synthesized in a variety of ways including, but not limited to, atom radical polymerization, reversible-addition fragmentation chain transfer polymerization, nitrous oxide-mediated polymerization, photo initiator-mediated polymerization, and can be selected based on the surface. For example, and in no way limiting, linkers can be selected from polyethylene glycol (PEGs), polyphosphazenes, polylactide, polyglycolide, polycaprolactone, poly(6-azidohexyl methacrylate), poly(2-bromoisobutyryloxyethyl methacrylate), poly(n-butyl methacrylate), poly(benzyl methacrylate), poly(cadmium methacrylate), poly(2-diethylaminoethyl methacrylate), poly(2,3-dihydroxypropyl methacrylate), poly(2-diisopropylaminoethyl methacrylate), poly(l-ethylene glycol dimethacrylate), poly(ethyl methacrylate), poly(3-ethyl-3-(methacryloyloxy methyloxetane), poly(ferrocenylmethyl methacrylate), poly(2-gluconamidoethyl methacrylate), poly(glycidyl methacrylate), poly(heptadecafluorodecyl methacrylate), poly(2-hydroxyethyl methacrylate), poly(2-hydroxylpropyl methacrylate), poly(isobutyl methacrylate), poly(isobornyl methacrylate), poly(2-lactobionamidoethyl methacrylate), poly(methacrylic acid), poly(methaacryloyladenosine), poly(3-O-methacryloly-di-Oisopropylidene-D-glucofuranose), poly(4-(10-methacryloydecyloxy)-4-pentylazobenzene), poly(2-methoxyethyl methacrylate), poly(2-(methacryloyloxy)ethyl succinate), poly(methyl methacrylate), poly(methacryloyluridine), poly(N-hydroxylsuccinimide methacrylate), poly(2-N-morpholinoethyl methacrylate), poly(octadecyl methacrylate), poly(poly(ethylene glycol) dimethacrylate, poly(poly(ethylene glycol) methacrylate, poly(poly(ethylene glycol)methyl ether methacrylate, poly(3-perylenylmethyl methacrylate, poly(2,2-dimethyl-1,3,-dioxolan-4-yl methyl methacrylate), poly(sprirobenzopyran methacrylate), poly(2-(tert-butylamino)ethyl methacrylate), poly(tert-butyl methacrylate), poly(trifluoroethyl methacrylate), poly(trimethylsilyl methacrylate), poly(3-(trimethoxylsilyl)propyl methacrylate), 2-(perfluoroalkyl)ethyl methacrylate, poly(2-(1-butylimidazolium-3-yl)ethyl methacrylate hexafluorophosphate, poly(carboxybetaine methacrylate), poly(l-ethyl 3-(2-methacryloyloxy ethyl) imidazolium chloride), poly(sodium methacrylate), poly(2-methacryloyloxyethyl phosphate), poly(2-(methacryloyloxy)ethyl phosphorylcholine), poly(sulfobetanine methacrylate), poly(2-sulfatoethyl methacrylate), poly(potassium 3-sulfopropyl methacrylate), poly(acrulic acid), poly(n-butyl acrylate), poly(2-bromoacetyloxyethyl acrylate), poly(2-(2-bromopropionyloxy)ethyl acrylate), poly(benzyl acrylate), poly(l1-(4-cyanophenyl-4-phenoxy)undecyl acrylate), poly(2-(dimethylamino)ethyl acrylate), poly(ethyl acrylate), poly(ethylene glycol diacrylate), poly(fluorescein acrylate), poly(l,6-hexanediol diacrylate), poly(heptadecafluorodecyl acrylate), poly(2-hydroxylethyl acrylate), poly(methyl acrylate), poly(octyl acrylate), poly(octadecyl acrylate), poly(poly(ethylene glycol)acrylate), poly(poly(glycol ethylene)acrylate succinyl fluorescein), poly(poly(glycol ethylene) methyl ether acrylate, poly(pentafluoropropyl acrylate), poly(tert-butyl acrylate), poly(trifluoroethyl acrylate), poly(trimethylsilyl acrylate), poly(triphenylamine acrylate), poly(N-(2-hydroxypropyl)methacrylamine, poly(methacrylamide), poly((3-methacryloylamino)propyl)-dimethyl-(3-sulfopropyl)ammonium hydroxide), poly(N-acryloyl glucosamine), poly(acrylamide), poly(potassium 2-acrylamido-2-methylpropane sulfonate), poly(carboxylbetane acrylamide), poly(N-cyclopropyl acrylamide), poly(N,N-dimethylacrylamide), poly(N-(3-(dimethylamino)propyl) acrylamide, poly(N-(3-dimethylamino)propyl) acrylamide methiodide), poly(N-hydroxylmethyl acrylamide), poly(N-N-methylenebisacrylamide), poly(methoxylethylacrylamide), poly(N-(6-(N-tert-butoxy-carbonylaminooxy)hexyl)acrylamide), poly(N-isopropyl acrylamide), poly(poly(ethylene glycol) methyl ether acrylamide), poly(acetoxystryrene), poly(4-chloromethylstyrene), poly(divinylbenzene), poly(4-(perfluoroalkyl)-oxymethylstyrene), poly(tert-butoxy-vinylbenzyl-polyglycidol), poly(4-methylstyrene), poly(N-octadecyl-N-(4-vinyl)-benzoyl-phenylalanineamide), polystyrene, poly(4-(poly(ethylene glycol) methyl ether styrene), poly(4-vinylaniline), poly(4-vinylbenzocyclobutene), poly(vinylquinoline), poly(4-styrenesulfonate), poly(4-vinylbenzoate), poly(l-(4-vinylbenzyl)-3-(butyl-imidazolium hexafluorophophate), poly(2-vinylpyridine), poly(3-vinylpyridine), poly(acrylonitrile), poly(itaconic acid), poly(maleic anhydride), poly(N-vinylimidazole), poly(N-vinyl-2-pyrrolidone), poly(N-vinyl-2-pyrrolidone), poly(m-isopropenyl-dimethyl-benzyl isocyanate), poly(2-vinyl-4,4-dimethyl azlactone), or any other combinations thereof. In some embodiments, combinations include any two or more of the aforementioned linkers attached at one end to a surface and at the other end with the QS modulating molecule. In other embodiments, combinations include any two or more of the aforementioned linkers arranged serially, e.g., a first linker having one end attached to a surface and another end attached to a second linker, the second linker having one end attached to the first linker and another end attached to a third linker, etc.). In other embodiments, any polymer architectures that consist of any combination of two or more of the aforementioned linkers including, but not limited to, end-functional linear polymers, di-end functional linear polymers, telechelic polymers, many-arm star polymers, copolymers, block polymers, dendritic polymers, branched polymers, gradient polymers, grafted polymers, microgel polymers, etc.

Any specific chemistry can be used to form a chemical bond between a surface (e.g., plastic/glass/gold substrates) and a linker. For example, silanization, gold-sulfide bond formation, thiol-ene reactions, surface-initiated polymerization, etc. can be used to form a bond between a surface and a linker. In a preferred embodiment, the linker functionalized with silane moieties at one terminus can be covalently bound to the surface via hydroxyl moieties on the glass substrate using silanization. In yet another preferred embodiment, the linker functionalized with maleimide moieties at one terminus can be covalently bound to —SH— moieties on the glass substrate via thiol-ene reactions. In still another preferred embodiment, the linker can be covalently bound to the surface by using surface-initiated polymerization to catalyze chemical reactions via -SH- moieties on the glass substrate. In yet another preferred embodiment, the linker functionalized with thiol moieties at one terminus can be covalently bound to the surface on a gold substrate via gold-sulfide bond formation, wherein the glass surface has been coated with a gold substrate.

According to embodiments of the present invention, a variety of specific chemistries can be used to form a chemical bond between a surface and a linker and/or between a linker and a QS modulating molecule. Thus, a variety of synthetic chemical strategies are available to create surface-attached molecules for manipulation of QS. Examples of specific chemistries include, but are not limited to, biorthogonal reactions, click chemistry, thiol-ene reactions, gold-sulfide bond formation, esterification reactions, Grignard reactions, Michael reactions, ketone/hydroxylamine condensations, Staudinger ligations, strain-promoted alkyne-azide cycloadditions, photo-click cycloadditions, Diels-Alder cycloadditions, tetrazine-alkene/alkyne cycloadditions, Cu-catalyzed alkyne-azide cycloadditions, Pd-catalyzed cross coupling, strain promoted alkyne-nitrone cycloadditions, Cross-metathesis, Norbornene cycloadditions, Oxanorbornadiene cycloadditions, tetrazine ligations, tetrazole photoclick chemistry, or any other combinations of these chemistries.

The present invention also relates to a method of screening a test compound that can modulate (i.e. reduce/inhibit or promote) QS, biofilm formation, biofilm streamer formation, and/or virulence factor production by a microorganism, by contacting the surface comprising an attached QS modulating molecule (i.e., antagonist or agonist) with the test compound and by measuring the modulation (i.e., reduction/elimination or promotion) of QS, biofilm formation, biofilm streamer formation, growth, and/or morphology changes. This method includes contacting a composition comprising a test compound attached to a surface through a linker as described herein and monitoring either: (1) the reduction and/or elimination of QS, biofilm formation, biofilm streamer formation, virulence factor production, growth, and/or morphology/phenotypic changes; or (2) the promotion and/or increase of QS, biofilm formation, biofilm streamer formation, virulence factor production, growth, and/or morphology/phenotypic changes. For example, by determining the time until clogging (T) and the duration of the clogging transition (τ) or by imaging the formation, growth or morphology/phenotypic changes of the biofilm and/or biofilm streamer and/or production of virulence factors, Alternatively, by measuring the expression of a fluorescently tagged quorum-sensing-repressed molecule such as GFPmut2 (indicating that QS is off) and/or by measuring the expression of a second fluorescently tagged quorum-sensing-activated molecule such as mKate2 (indicating that QS is on), one can readily determine the ability of a test compound attached to a surface through a linker as described herein to inhibit or enhance biofilm and/or biofilm streamer growth. Other embodiments including a method of screening test compounds to identify compounds that can inhibit, promote or affect biofilm and/or biofilm streamer formation are also contemplated.

Bacterial organisms may be Gram-positive or Gram-negative. Gram-positive bacteria have a peptidoglycan coating covering the bacterial cell membrane. Thus, in some embodiments, the linker is of a sufficient length to traverse the peptidoglycan coating in order to interact with the receptor on the underlying cell membrane. In some embodiments, the peptidoglycan coating may have a thickness ranging from about 1 nm to about 200 nm, from about 1 nm to about 100 nm, from about 1 nm to about 50 nm, from about 15 nm to about 50 nm, from about 15 nm to about 30 nm, or from about 1 nm to about 15 nm.

The length of the linker may be designed to be longer than the peptidoglycan coating, so that the QS modulating molecule is able to interact with the receptor on the bacterial cell membrane. As the peptidoglycan coating thickness may vary between different species of bacteria, the length of the linker may vary depending on the bacterial organism targeted. Therefore, in some embodiments, the linker may have a length between 1 nm and about 200 nm, between about 1 nm to about 100 nm, between about 1 nm to about 50 nm, between about 15 nm to about 50 nm, between about 15 nm to about 30 nm, or between about 1 nm to about 15 nm. As an example, the linker PEG₁₀₀₀₀ is of a sufficient length to interact with cell membrane receptors for S. aureus.

In other embodiments, the linker may have a length greater than 1 nm, 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, and 20 nm.

Gram-negative bacteria have an outer membrane layer covering a peptidoglycan layer. In some embodiments, the Gram-negative bacteria are treated with a reagent to make the outer membrane layer porous prior to coming into contact with the surface attached QS modulating molecules. In this embodiment, the length of the linker should be greater than the thickness of the outer membrane layer plus the thickness of the peptidoglycan layer, so that the QS modulatory molecule is able to interact with the receptor on the bacterial inner-cell membrane.

The peptidoglycan layer is an elastic mesh network with pores. In some embodiments, the pores may range in diameter from about 1 nm to 5 nm, from about 1 nm to about 10 nm, from about 1 nm to about 20 nm, or from about 1 nm to about 50 nm. Accordingly, the diameter of the linker may be designed to be smaller than the width of the peptidoglycan pores. Similarly, for Gram-negative bacteria that have been treated with a reagent to make the outer cell layer porous, the diameter of the linker may be desgined to be smaller than the diameter of the pore of the outer cell layer or of the peptidoglycan matrix, whichever is smaller. In some embodiments, the diameter of the linker is less than 5 nm, 6 nm, 7 nm, 8 nm, 9 nm or 10 nm.

The surface density of the QS modulating molecule is a factor in determining whether the QS modulating molecule can affect the behavior of the bacteria. In some embodiments, the average surface coverage density of the QS modulating molecule is between about 2×10² μm⁻² and 2×10⁴ μm⁻². In preferred embodiments, the surface attached QS modulating molecule is 2.1×10² μm⁻² or greater. In some embodiments, the average surface coverage density of the QS modulating molecule is between about 2×10⁰ μm⁻² and 2×10² μm⁻² or between about 2×10⁻² μm⁻² and 2×10⁰ μm⁻².

In still other embodiments, the surface may comprise multiple types of QS modulating molecules e.g., two or more antagonists, two or more agonists, or a combination of antagonists and agonists. Each agonist or antagonist may be attached to the surface via a linker. For example, a first agonist may be tethered to the surface with a first type of linker and a second agonist may be tethered to the surface with a second type of linker. In some embodiments, two or more QS modulating molecules may be attached to the surface with the same type of linker. In other embodiments, a QS modulating molecule may be attached to the surface with the two or more types of linkers.

In still other embodiments, the surfaces having surface attached QS modulating molecules are stable. For example, the surfaces may be stored for up to 40 days, 50 days, 60 days, 70 days, or 80 days at about 4° C. without undergoing significant degradation.

In some embodiments, the QS modulating molecules are attached to the surface of a device (e.g., a medical device, a device to prevent biofouling, etc.). Due to the stability of the surface attached QS modulating molecules, the devices may be exposed to repeat infections in a serial manner and still retain the capability to modulate QS behavior of the bacteria. The surface attached QS modulating molecules are also resistant to degradation by host plasma components and to flow-generated stresses, e.g., from fluid entering and exiting the device. Thus, the surface attached QS modulating molecules described herein exhibit superior stability under a variety of conditions.

Embodiments of the present invention also relate to a method of detecting specific microorganisms that can respond to specific QS modulating molecules. An unknown microorganism that contacts the QS modulating molecule attached surface and responds to the surface will undergo a change in QS phenotype, and this alteration can be used to detect particular types of microorganisms. In one embodiment, a sample that contains an unknown bacterium that causes an infection in a patient in a healthcare setting can be introduced onto the QS modulating molecule-coated surface, and an alteration in a QS phenotype can be measured. In another embodiment, a sample that contains an unknown bacterium that causes contamination in a food processing setting can be introduced onto the QS modulating molecule-coated surface, and an alteration in a QS phenotype, such as byproduct production, or other traits can be measured. This application can be more rapid and provide lower detection limits than conventional microbial detection methodologies such as PCR verification techniques, immunological methods, and amplification methods in use today, which could be important to treat severely ill patients.

Thus, in preferred embodiments, the invention is described as a surface comprising a quorum sensing (“QS”) modulating molecule attached to the surface by a linker. In preferred embodiments: (a) the length of the linker is sufficient to traverse a bacterial cell's peptidoglycan layer, outer membrane layer or both; (b) the QS modulating molecule binds to a receptor on a cell membrane of a bacterial cell; (c) the linker has a diameter of less than 5 nm; (d) the linker has a length greater than 1, 2, 3, 4, 5, 6, 7, 8, 9, 10, 11, 12, 13, 14, 15 nm; (e) the linker is a chemical bound; (f) the linker is selected from polyethylene glycol (PEGs), polyphosphazenes, polylactide, polyglycolide, polycaprolactone, or any other combinations thereof; (g) the linker is attached to the surface using one or more of the following types of chemical reactions: silanization, gold-sulfide bond formation, thiol-ene reactions, and surface-initiated polymerization; (h) the average surface coverage density of the QS modulating molecule is of a sufficient density to modulate QS; (i) the average surface coverage density of the QS modulating molecule is about 2.1×10² μm⁻² or greater; (j) the QS modulating molecule comprises an antagonist of QS that alters QS-controlled phenotypes of biofilm production, biofilm streamer production, and/or virulence factor production; (k) the QS modulating molecule comprises an agonist of QS that alters QS-controlled phenotypes of biofilm production, biofilm streamer production and/or virulence factor production; (l) the QS modulating molecule retains modulating activity after being stored at about 4° C. for up to 40 days; (m) the QS modulating molecule remains bound to the surface via the linker after exposure to laminar fluid flow; (n) the QS modulating molecule exhibits modulating activity when exposed to a population of bacterial cells; (o) the QS modulating molecule exhibits modulating activity when reexposed to another population of bacterial cells; (p) the QS modulating molecule is a molecule selected from Tables 1A or 1B or its derivative molecules; (q) the QS modulating molecule is attached to the linker using one or more of the following types of chemical reactions: biorthogonal reactions, click chemistry, thiol-ene reactions, gold-sulfide bond formation, esterification reactions, Grignard reactions, Michael reactions, ketone/hydroxylamine condensations, Staudinger ligations, strain-promoted alkyne-azide cycloadditions, photo-click cycloadditions, Diels-Alder cycloadditions, tetrazine-alkene/alkyne cycloadditions, Cu-catalyzed alkyne-azide cycloadditions, Pd-catalyzed cross coupling, strain promoted alkyne-nitrone cycloadditions, Cross-metathesis, Norbornene cycloadditions, Oxanorbornadiene cycloadditions, tetrazine ligations, or tetrazole photoclick chemistry; (r) the surface comprises glass, metal, stainless metal, silicon, plastic, polymer, metal, or ceramic material or any combination thereof; (s) the surface is a small particle, a nanoparticle, a flat surface or a curved surface; or (t) any combination of (a)-(s).

In preferred embodiments, the bacterial cell is Gram-negative, Gram-positive or a mixture of Gram-negative and Gram-positve. In further preferred embodiments, the bacterial cell is exposed to a permeability agent that forms holes in the outer membrane layer of the bacterial cell prior to contacting the surface.

Additional preferred embodiments include (a) the polymer is selected from polyethylene, polypropylene, polystyrene, polyester, polyester PLA and other biosorbable plastics, polycarbonate, polyvinyl chloride, polyethersulfone, polyacrylate (e.g., Acrylic, PMMA), hydrogel (e.g., acrylate), polysulfone, polyetheretherketone, thermoplastic elastomers (e.g., TPE, TPU), thermoset elastomers, silicone, poly-p-xylylene (e.g., Parylene), fluoropolymers; (b) the metal is selected from stainless steel, cobalt-base alloy, titanium, titanium-base alloy, and/or shape memory alloy; and/or (c) the ceramic material comprises glass ceramic, calcium phosphate ceramic, and/or carbon-based ceramic.

In further preferred embodiments, the surface comprises a second QS modulating molecule attached the surface by a second linker. In these situations, preferred embodiments include, (a) the length of the second linker is sufficient to traverse the bacterial cell's peptidoglycan layer, outer membrane layer or both; (b) the second QS modulating molecule competitively binds to the receptor on the cell membrane of the bacterial cell; (c) wherein the second QS modulating molecule binds to a different receptor on the cell membrane; (d) the second linker has a diameter of less than 5 nm; (e) the second linker has a length greater than 1, 2, 3, 4, 5, 6, 7, 8, 9, 10, 11, 12, 13, 14, or 15 nm; (f) the second linker is a chemical bound; (g) the second linker is selected from polyethylene glycol (PEGs), polyphosphazenes, polylactide, polyglycolide, polycaprolactone, or any other combinations thereof; (h) the second linker is attached to the surface using one or more of the following types of chemical reactions: silanization, gold-sulfide bond formation, thiol-ene reactions, and surface-initiated polymerization; (i) the second linker is the same as the linker; (j) the second linker is different from the linker; (k) any combination of (a)-(j).

In preferred embodiments, the surface is placed in an environment, and preferably the environment is (a) static; (b) under pressure; (c) a flow environment; (d) under controlled pressure; and/or (e) an implantable medical device, part of machinery used in industrial processes, a culvert, a pool used in a waste water treatment facility, waste water treatment facility, a pipe, a cooling tower, a medical device, industrial fluid handling machinery, a wound, within the body, a medical process, an agricultural process, and/or machinery.

In further preferred embodiments, the invention is a method of modulating QS, biofilm formation, biofilm streamer formation, and/or a virulence factor production by a microorganism, wherein the method comprises contacting a microorganism with the surface described herein. For example, the method inhibits a pathogenic behavior of a microorganism and/or promotes a beneficial behavior of a microorganism.

Examples of microorganism that can be modulated include, but are not limited to bacteria, archaea, protozoa, fungi, and/or algae. Further examples of strains of bacteria include but are not limited to Abiotrophia, Achromobacter, Acidaminococcus, Acidovorax, Acinetobacter, Actinobacillus, Actinobaculum, Actinomadura, Actinomyces, Aerococcus, Aeromonas, Afipia, Agrobacterium, Alcaligenes, Alloiococcus, Alteromonas, Amycolata, Amycolatopsis, Anabaena, Anabaenopsis, Anaerobospirillum, Anaerorhabdus, Aphanizomenon, Arachnia, Arcanobacterium, Arcobacter, Arthrobacter, Atopobium, Aureobacterium, Bacillus, Bacteroides, Balneatrix, Bartonella, Bergeyella, Bifidobacterium, Bilophila, Bordetella, Borrelia, Brachyspira, Branhamella, Brevibacillus, Brevibacterium, Brevundimonas, Brucella, Burkholderia, Buttiauxella, Butyrivibrio, Calymmatobacterium, Camesiphon, Campylobacter, Capnocytophaga, Capnylophaga, Cardiobacterium, Catonella, Cedecea, Cellulomonas, Centipeda, Chlamydia, Chlamydophila, Chromobacterium, Chryseomonas, Chyseobacterium, Citrobacter, Clostridium, Collinsella, Comamonas, Corynebacterium, Coxiella, Cryptobacterium, Cyanobacteria, Cylindrospermopsis, Delftia, Dermabacter, Dermatophilus, Desulfomonas, Desulfovibrio, Dialister, Dichelobacter, Dolosicoccus, Dolosigranulum, Edwardsiella, Eggerthella, Ehrlichia, Eikenella, Empedobacter, Enterobacter, Enterococcus, Erwinia, Erysipelothrix, Escherichia, Eubacterium, Ewingella, Exiguobacterium, Facklamia, Filifactor, Flavimonas, Flavobacterium, Francisella, Fusobacterium, Gardnerella, Gemella, Globicatella, Gloeobacter, Gordona, Haemophilus, Hafnia, Hapalosiphon, Helicobacter, Helococcus, Hemophilus, Holdemania, Ignavigranum, Johnsonella, Kingella, Klebsiella, Kocuria, Koserella, Kurthia, Kytococcus, Lactobacillus, Lactococcus, Lautropia, Leclercia, Legionella, Leminorella, Leptospira, Leptospirae, Leptotrichia, Leuconostoc, Listeria, Listonella, Lyngbya, Megasphaera, Methylobacterium, Microbacterium, Micrococcus, Microcystis, Mitsuokella, Mobiluncus, Moellerella, Moraxella, Morganella, Mycobacterium, Mycoplasma, Myroides, Neisseria, Nocardia, Nocardiopsis, Nodularia, Nostoc, Ochrobactrum, Oeskovia, Oligella, Orientia, Paenibacillus, Pantoea, Parachlamydia, Pasteurella, Pediococcus, Peptococcus, Peptostreptococcus, Phormidium, Photobacterium, Photorhabdus, Phyllobacterium, Phytoplasma, Planktothrix, Plesiomonas, Porphyromonas, Prevotella, Propionibacterium, Proteus, Providencia, Pseudoanabaena, Pseudomonas, Pseudonocardia, Pseudoramibacter, Psychrobacter, Rahnella, Ralstonia, Rhodococcus, Rickettsia, Rochalimaea, Roseomonas, Rothia, Ruminococcus, Salmonella, Schizothrix, Selenomonas, Serpulina, Serratia, Shewenella, Shigella, Simkania, Slackia, Sphaerotilus, Sphingobacterium, Sphingomonas, Spirillum, Spiroplasma, Spirulina, Staphylococcus, Stenotrophomonas, Stomatococcus, Streptobacillus, Streptococcus, Streptomyces, Succinivibrio, Sutterella, Suttonella, Tatumella, Tissierella, Trabulsiella, Treponema, Trichodesmium, Tropheryma, Tsakamurella, Turicella, Umezakia, Ureaplasma, Vagococcus, Veillonella, Vibrio, Weeksella, Wolinella, Xanthomonas, Xenorhabdus, Yersinia, Yokenella. Acinetobacter baumannii, Actinobacillus actinomycetemcomitans, Actinobacillus pleuropneumoniae, Actinomyces bovis, Actinomyces Israelii, Bacillus anthracis, Bacillus ceretus, Bacillus coagulans, Bacillus liquefaciens, Bacillus popillae, Bacillus subtilis, Bacillus thuringiensis, Bacteroides distasonis, Bacteroides fragilis, Bacteroides thetaiotaomicron, Bacteroides vulgatus, Bartonella bacilliformis, Bartonella Quintana, Beneckea parahaemolytica, Bordetella bronchiseptica, Bordetella parapertussis, Bordetella pertussis, Borelia burgdorferi, Brevibacterium lactofermentum, Brucella abortus, Brucella canis, Brucella melitensis, Brucella suis, Burkholderia cepacia, Burkholderia mallei, Burkholderia pseudomallei, Campylobacter fetus, Campylobacter jejuni, Campylobacter pylori, Cardiobacterium hominis, Chlamydia pneumoniae, Chlamydia psittaci, Chlamydia trachomatis, Chlamydophila abortus, Chlamydophila caviae, Chlamydophila felis, Chlamydophila pneumonia, Chlamydophila psittaci, Chryseobacterium eningosepticum, Clostridium botulinum, Clostridium butyricum, Clostridium coccoides, Clostridium dijficile, Clostridium leptum, Clostridium tetani, Corynebacterium xerosis, Cowdria ruminantium, Coxiella burnetii, Edwardsiella tarda, Ehrlichia sennetsu, Eikenella corrodens, Elizabethkingia meningoseptica, Enterobacter aerogenes, Enterobacter cloacae, Enterococcus faecalis, Escherichia coli, Escherichia hirae, Flavobacterium meningosepticum, Fluoribacter bozemanae, Francisella tularensis, Francisella tularensis biovar Tularensis, Francisella tularensis subsp. Holarctica, Francisella tularensis subsp. nearctica, Francisella tularensis subsp. Tularensis, Francisella tularensis var. palaearctica, Fudobascterium nucleatum, Fusobacterium necrophorum, Haemophilus ducreyi, Haemophilus influenzae, Helicobacter pylori, Kingella kingae, Klebsiella mobilis, Klebsiella oxytoca, Klebsiella pneumoniae, Lactobacillus acidophilus, Lactobacillus casei, Lactobacillus hilgardii, Lactobacillus pentosus, Lactobacillus plantarum, Lactobacillus rhamnosus, Lactococcus lactis, Legionella bozemanae corrig., Legionella pneumophila, Leptospira alexanderi, Leptospira borgpetersenii, Leptospira fainei, Leptospira inadai, Leptospira interrogans, Leptospira kirschneri, Leptospira noguchii, Leptospira santarosai, Leptospira weilii, Leuconostoc lactis, Leuconostoc oenos, Listeria ivanovii, Listeria monocytogenes, Moraxella catarrhalis, Morganella morganii, Mycobacterium africanum, Mycobacterium avium, Mycobacterium avium subspecies paratuberculosis, Mycobacterium bovis, Mycobacterium bovis strain BCG, Mycobacterium intracellulare, Mycobacterium kansasii, Mycobacterium leprae, Mycobacterium marinum, Mycobacterium tuberculosis, Mycobacterium typhimurium, Mycobacterium ulcerans, Mycoplasma hominis, Mycoplasma mycoides, Mycoplasma pneumoniae, Neisseria gonorrhoeae, Neisseria meningitidis, Neorickettsia sennetsu, Nocardia asteroides, Orientia tsutsugamushi, Pasteurella haemolytica, Pasteurella multocida, Plesiomonas shigelloides, Propionibacterium acnes, Proteus mirabilis, Proteus morganii, Proteus penneri, Proteus rettgeri, Proteus vulgaris, Providencia akalifaciens, Providencia rettgeri, Pseudomonas aeruginosa, Pseudomonas mallei, Pseudomonas pseudomallei, Pyrococcus abyssi, Rickettsia akari, Rickettsia canadensis, Rickettsia canadensis corrig, Rickettsia conorii, Rickettsia montanensis, Rickettsia montanensis corrig, Rickettsia prowazekii, Rickettsia rickettsii, Rickettsia sennetsu, Rickettsia tsutsugamushi, Rickettsia typhi, Rochalimaea quintana, Salmonella arizonae, Salmonella choleraesuis subsp. arizonae, Salmonella enterica subsp. Arizonae, Salmonella enteritidis, Salmonella paratyphi, Salmonella typhi, Salmonella typhimurium, Selenomonas nominantium, Selenomonas ruminatium, Serratia marcescens, Shigella boydii, Shigella dysenteriae, Shigella flexneri, Shigella sonnei, Spirillum minus, Staphylococcus aureus, Staphylococcus epidermidis, Staphylococcus equi, Staphylococcus lugdunensis, Stenotrophomonas maltophila, Streptobacillus moniliformis, Streptococcus agalactiae, Streptococcus bovis, Streptococcus ferus, Streptococcus pneumoniae, Streptococcus pyogenes, Streptococcus viridans, Streptomyces ghanaenis, Streptomyces hygroscopicus, Streptomyces phaechromogenes, Treponema carateum, Treponema denticola, Treponema pallidum, Treponema pertenue, Vibrio cholerae, Vibrio parahaemolyticus, Vibrio vulnificus, Xanthomonas maltophilia, Yersinia enterocolitica, Yersinia pestis, Yersinia pseudotuberculosis, Zymomonas mobilis, or Fusospirochetes.

Examples of fungi include, but are not limited to Candida, Saccharomyces, or Cryptococcus.

The invention is further directed to use of the surfaces described herein to treat sepsis, pneumonia, infections from cystic fibrosis, otitis media, chronic obstructive pulmonary disease, a urinary tract infection, periodontal disease, gingivitis, periodontitis, breath malodor, treat infections, Gram-negative infections, Gram-positive infections, otitis media, prostatitis, cystitis, bronchiectasis, bacterial endocarditis, osteomyelitis, dental caries, periodontal disease, infectious kidney stones, acne, Legionnaire's disease, chronic obstructive pulmonary disease (COPD), cystic fibrosis, an accumulation of biofilm in the lungs or digestive tract, emphysema, chronic bronchitis, also encompasses infections on implanted/inserted devices, medical device-related infections, biliary stent infections, orthopedic implant infections, catheter-related infections, skin infections, dermatitis, ulcers from peripheral vascular disease, a burn injury, trauma, rosacea, skin infection, pneumonia, otitis media, sinusitus, bronchitis, tonsillitis, and mastoiditis related to infection by Streptococcus pneumoniae, Haemophilus influenzae, Moraxella catarrhalis, Staphylococcus aureus, Peptostreptococcus spp. or Pseudomonas spp.; pharynigitis, rheumatic fever, and glomerulonephritis related to infection by Streptococcus pyogenes, Groups C and G streptococci, Clostridium diptheriae, or Actinobacillus haemolyticum; respiratory tract infections related to infection by Mycoplasma pneumoniae, Legionella pneumophila, Streptococcus pneumoniae, Haemophilus influenzae, or Chlamydia pneumoniae; uncomplicated skin and soft tissue infections, abscesses and osteomyelitis, and puerperal fever related to infection by Staphylococcus aureus, coagulase positive staphylococci (i.e., S. epidermidis, S. hemolyticus, etc.), S. pyogenes, S. agalactiae, Streptococcal groups C-F (minute-colony streptococci), viridans streptococci, Corynebacterium spp., Clostridium spp., or Bartonella henselae; uncomplicated acute urinary tract infections related to infection by S. saprophyticus or Enterococcus spp.; urethritis and cervicitis; sexually transmitted diseases related to infection by Chlamydia trachomatis, Haemophilus ducreyi, Treponema pallidum, Ureaplasma urealyticum, or Nesseria gonorrheae; toxin diseases related to infection by S. aureus (food poisoning and Toxic shock syndrome), or Groups A, S, and C streptococci; ulcers related to infection by Helicobacter pylori; systemic febrile syndromes related to infection by Borrelia recurrentis; Lyme disease related to infection by Borrelia burgdorferi; conjunctivitis, keratitis, and dacrocystitis related to infection by C. trachomatis, N gonorrhoeae, S. aureus, S. pneumoniae, S. pyogenes, H influenzae, or Listeria spp.; disseminated Mycobacterium avium complex (MAC) disease related to infection by Mycobacterium avium, or Mycobacterium intracellulare; gastroenteritis related to infection by Campylobacter jejuni; odontogenic infection related to infection by viridans streptococci; persistent cough related to infection by Bordetella pertussis; gas gangrene related to infection by Clostridium perfringens or Bacteroides spp.; skin infection by S. aureus, Propionibacterium acne; atherosclerosis related to infection by Helicobacter pylori or Chlamydia pneumoniae; or the like.

The surfaces described herein can also be used in a method of screening for a compound that modulates QS, biofilm formation, biofilm streamer formation, and/or a virulence factor production by a microorganism, wherein the method comprises contacting a compound with any one of the surfaces described herein and measuring whether QS, biofilm formation, biofilm streamer formation, and/or a virulence factor production by a microorganism is either increased, decreased or maintained.

### BRIEF DESCRIPTION OF THE DRAWINGS

Embodiments are illustrated by way of example and are not limited to the figures of the accompanying drawings, in which like references indicate similar elements and in which:

FIG. 1a. illustrates the Agr QS responses of S. aureus to exogenously supplied agonists and antagonists. To follow the QS status of S. aureus cells growing on unmodified and chemically-modified surfaces, confocal microscopy and an S. aureus reporter strain that produced the fluorescent protein mKate2 in response to exogenous addition of AIP-I was used. The reporter strain was a ΔagrBDCA ΔRNAIII S. aureus mutant harboring a multicopy plasmid carrying agrCA driven by the native agrP2 promoter. Thus, the strain did not produce endogenous AIP-I but plasmid restoration of AgrC-I and AgrA endowed the strain with the capability to detect AIP-I if it was supplied exogenously. The plasmid also harbored the Agr-activated agrP3 promoter fused to mkate2. Therefore, in response to exogenously provided AIP-I, the reporter strain fluoresced red. This QS response was repressed by the administration of TrAIP-II. A constitutively expressed sarAP1-gfpmut2 gene was introduced into the chromosome to enable normalization of QS responses¹⁸. FIG. 1b. To demonstrate that the S. aureus reporter strain faithfully reported on Agr QS, it was incubated in the absence and presence of 100 nM AIP-I (i.e., the agonist). GFPmut2 was produced irrespective of the presence of AIP-I, however, mKate2 was produced only when AIP-I was supplied to the cells (FIG. 1b). When the reporter strain was simultaneously provided with 100 nM AIP-I and 2.5 μM TrAIP-II (i.e., the antagonist), little mKate2 production occurred, indicating inhibition of Agr QS (FIG. 1c (ii)). Reducing the TrAIP-II antagonist concentration by 10-fold caused a corresponding 9-fold decrease in QS inhibition (FIG. 1c (v)). Addition of TrAIP-II alone elicited no mKate2 production (FIG. 1c (viii)).

FIG. 1b. Fluorescence images of the S. aureus reporter strain (pathways shown in panel FIG. 1a) after 3 h incubation with buffer or buffer containing 100 nM AIP-I agonist. Left panels (i) and (iv): constitutive expression of the sarAPl-gfpmut2 reporter. Middle panels (ii) and (v): expression of the QS-controlled agrP3-mkate2 reporter. Right panels (iii) and (vi): merged images from the left and middle panels. Top panels (i), (ii), and (iii) show incubation of the reporter strain with buffer. Bottom panels (iv), (v), and (vi) show incubation of the reporter strain with 100 nM AIP-I. 1c. Fluorescence images of the S. aureus reporter strain used in this study following 3 h incubation with TrAIP-II or with combinations of AIP-I and TrAIP-II. Left panels (i), (iv), and (vii): constitutive expression of the sarAP1-gfpmut2 reporter. Middle panels (ii), (v), and (viii): expression of the QS-controlled agrP3-mkate2 reporter. Right panels (iii), (vi), and (ix): merged images from the left and middle panels. Top panels (i), (ii), and (iii) show incubation of the reporter strain with 2.5 μM TrAIP-II+100 nM AIP-I. Middle panels (iv), (v), and (vi) show incubation of the reporter strain with 250 nM TrAIP-II+100 nM AIP-I. Bottom panels (vii), (viii), and (ix) show incubation of the reporter strain with 2.5 μM TrAIP-II. In panels FIG. 1b and 1c, images are based on n=3 independent replicates. Scale bars: 20 μm.

FIG. 2 illustrates activation of S. aureus Agr QS by surface-attached AIP-I. FIG. 2a. Strategy for modification of AIP-I and attachment to the surface; (i) AIP-I, (ii) Alkyne-AIP-I, (iii) PEG₃₃₀-triazole-AIP-I, and (iv) Surface-PEG₁₀₀₀₀-triazole-AIP-I. The PEG subscript denotes the molecular weight. FIG. 2b. The normalized S. aureus Agr QS output, which was defined as the QS-controlled reporter output divided by the output from the constitutively expressed reporter, in response to AIP-I (circles), Alkyne-AIP-I (squares), PEG₃₃₀-triazole-AIP-I (triangles), AIP-I+2.5 μM TrAIP-II (asterisks), Alkyne-AIP-I+2.5 μM TrAIP-II (diamonds), and PEG₃₃₀-triazole-AIP-I+2.5 μM TrAIP-II (upside down triangles). Data points indicate means, and error bars denote standard deviations from triplicate experiments. 2c. The normalized Agr QS output from the S. aureus reporter strain was measured as a function of time in the presence of Surface-PEG₁₀₀₀₀-triazole-AIP-I (asterisks), Surface-PEG₁₀₀₀₀-triazole-AIP-I+2.5 μM TrAIP-II in solution (diamonds), Surface-PEG₁₀₀₀₀ (squares), Surface-PEG₁₀₀₀₀-azide (circles), and Surface-PEG₁₀₀₀₀-triazole-Linear-AIP-I (triangles). See FIG. 10 for details of the chemical procedures in each panel. Normalized QS outputs were measured as means from 1000-4000 individual cells in each experiment. Data points indicate means, and error bars denote standard deviations from triplicate experiments. An ANOVA test with Tukey-Kramer post hoc analysis was used to assess the statistical significance between the means for the AIP-I coated surface and the control surfaces using the T=6 h data (**** P<0.0001). FIG. 2d. Representative merged fluorescence images of the S. aureus reporter strain on the Surface-PEG₁₀₀₀₀-triazole-AIP-I at T=0, 2, 4, and 6 h. Images were based on n=3 independent experiments; one representative image for each condition was chosen from ˜50 images acquired from different regions of each surface. Scale bars: 20 μm. AU: arbitrary units.

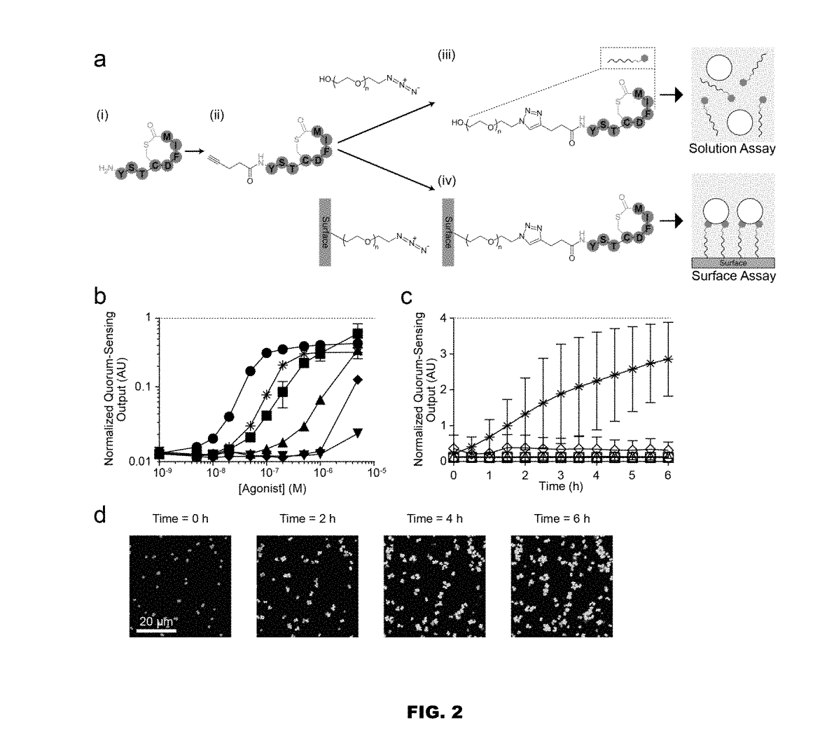

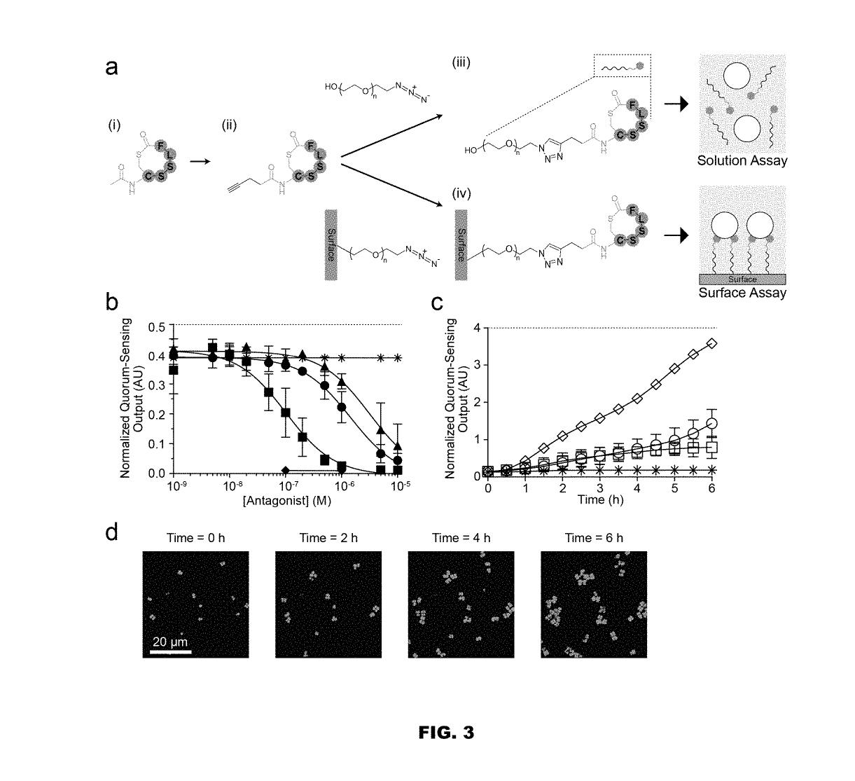

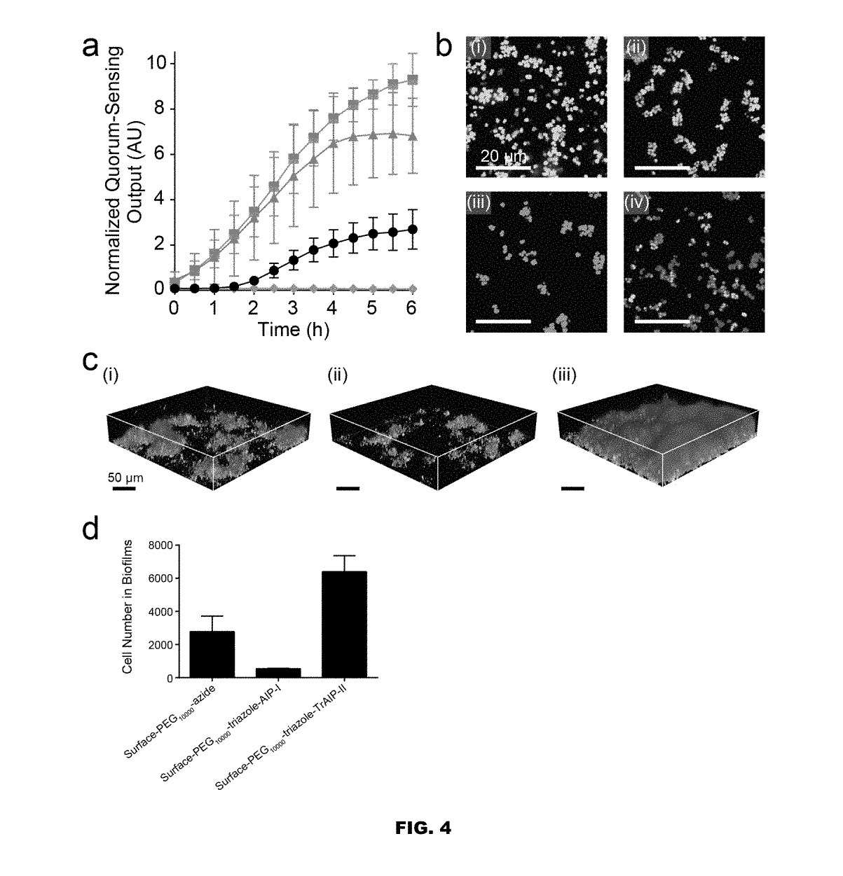

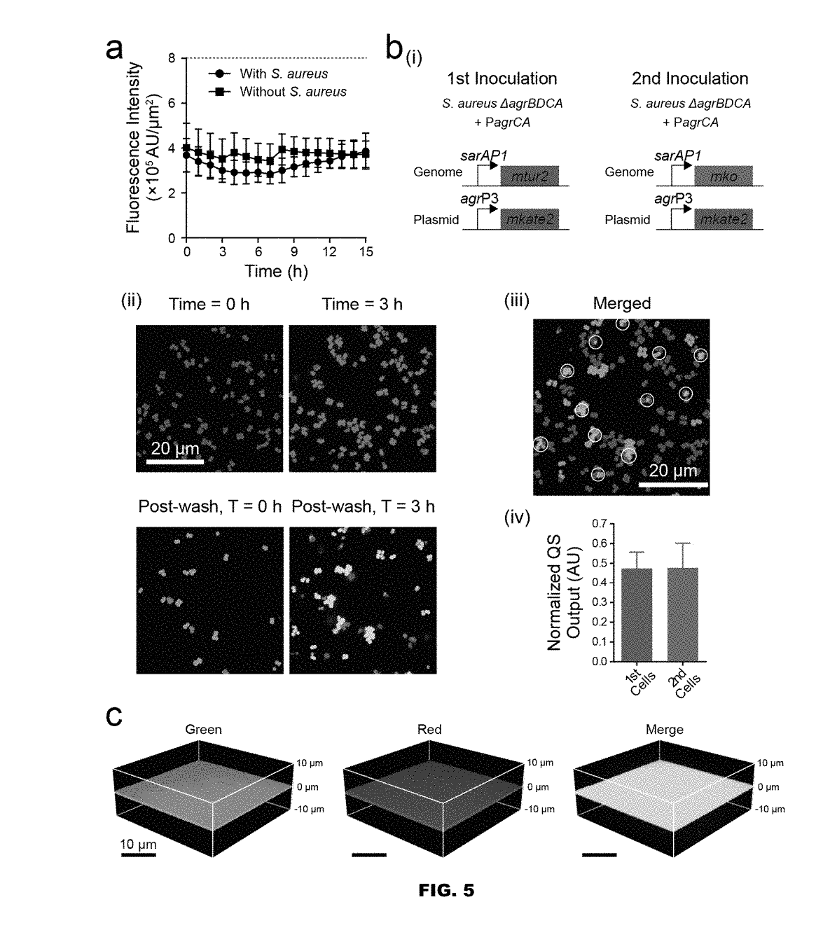

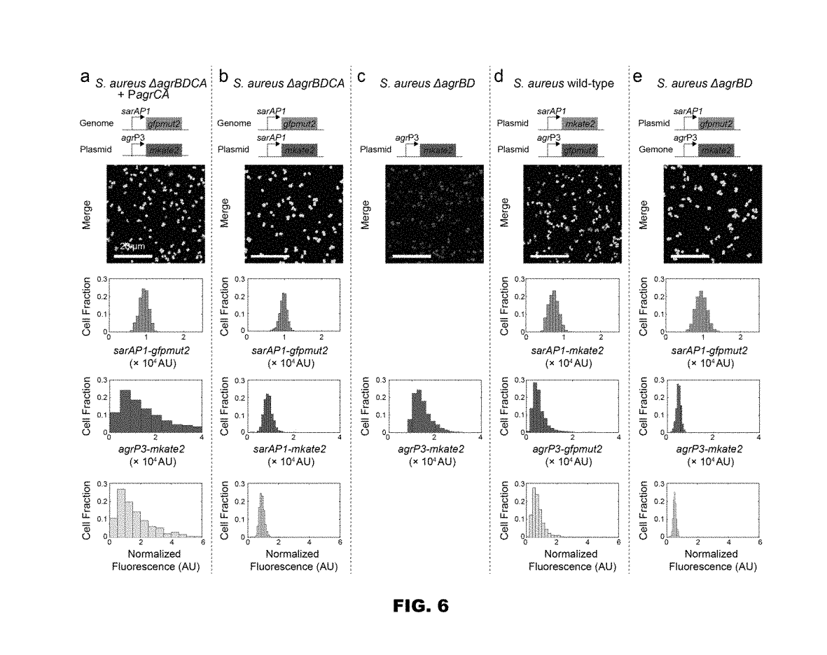

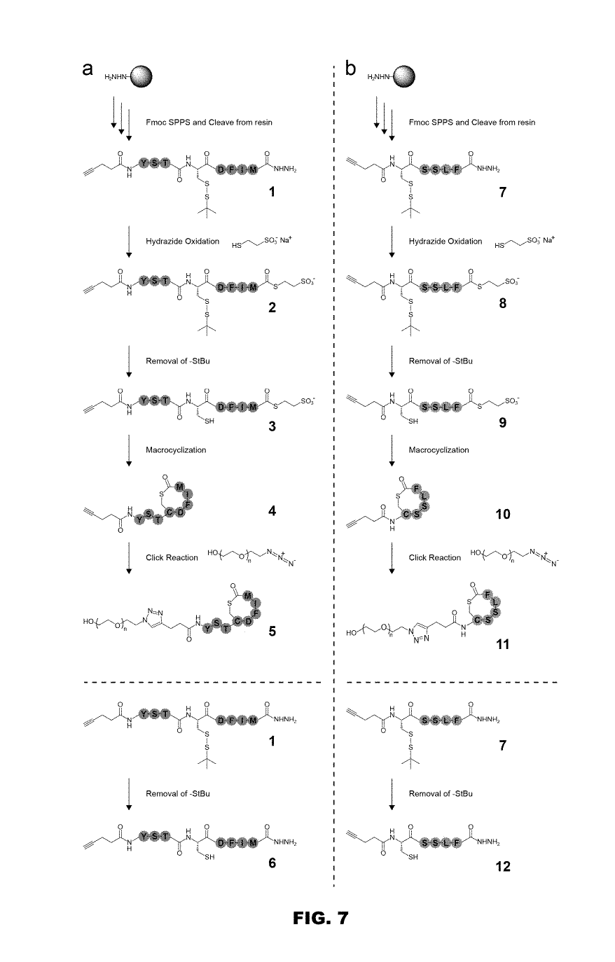

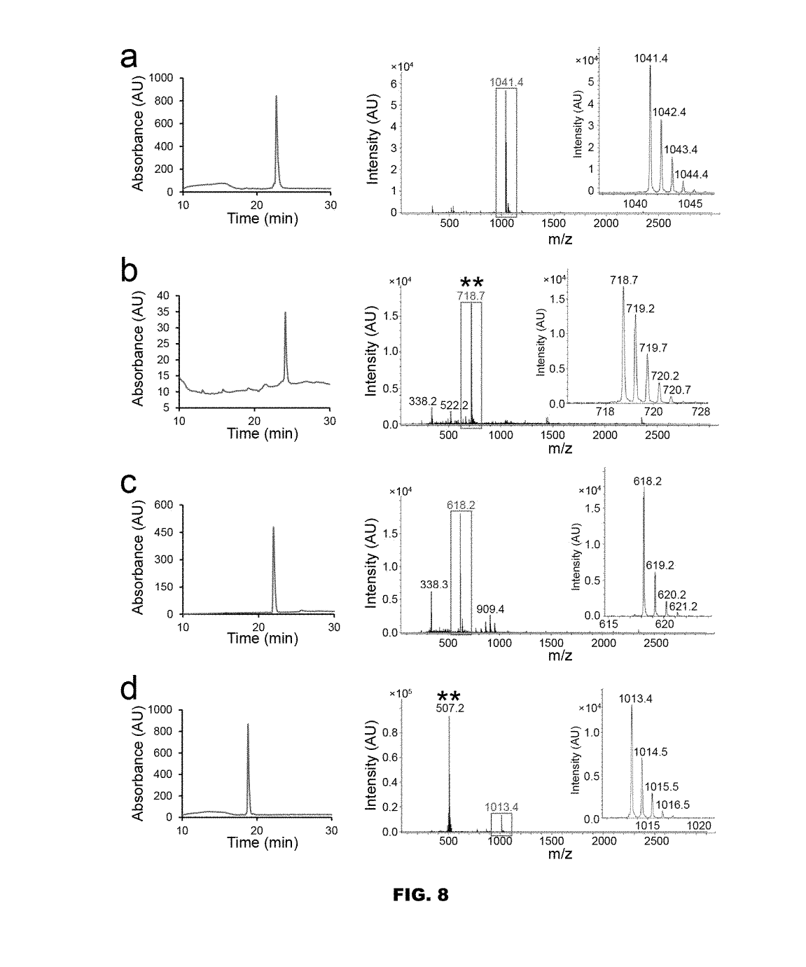

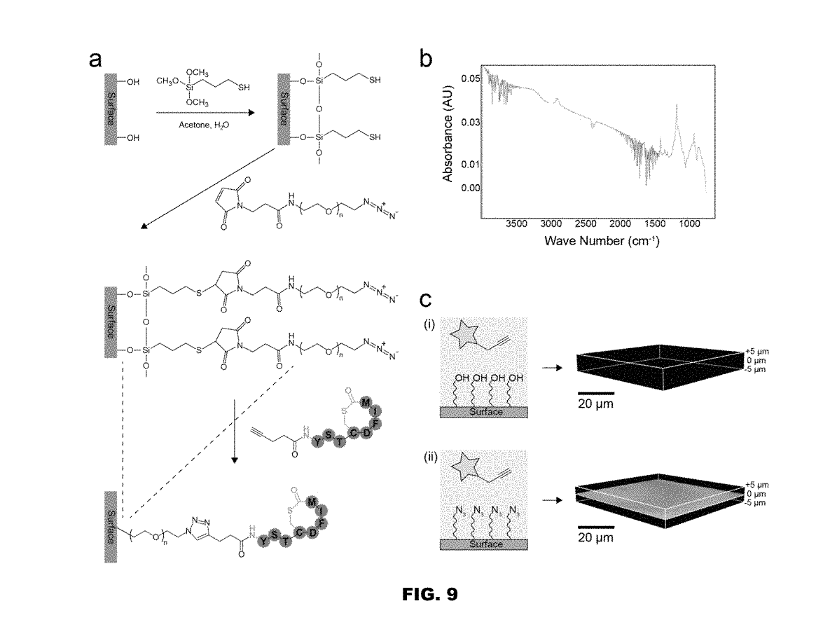

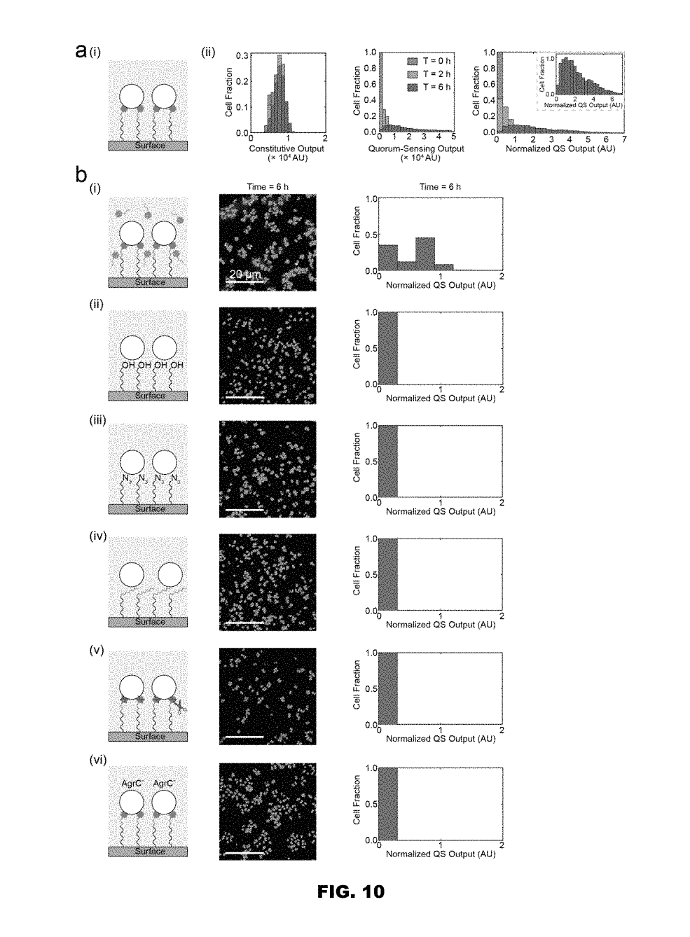

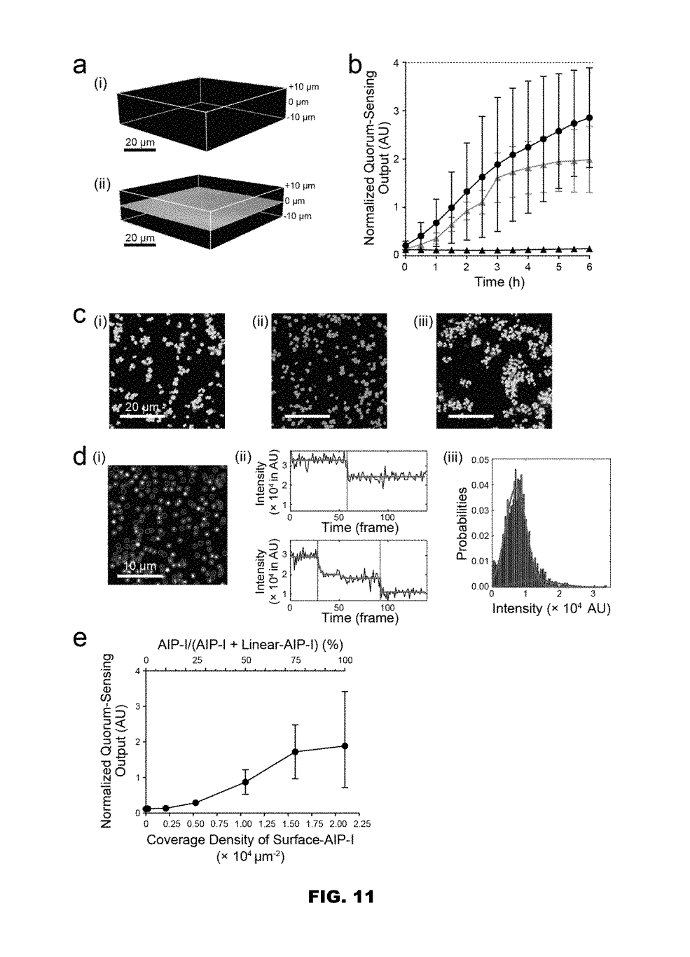

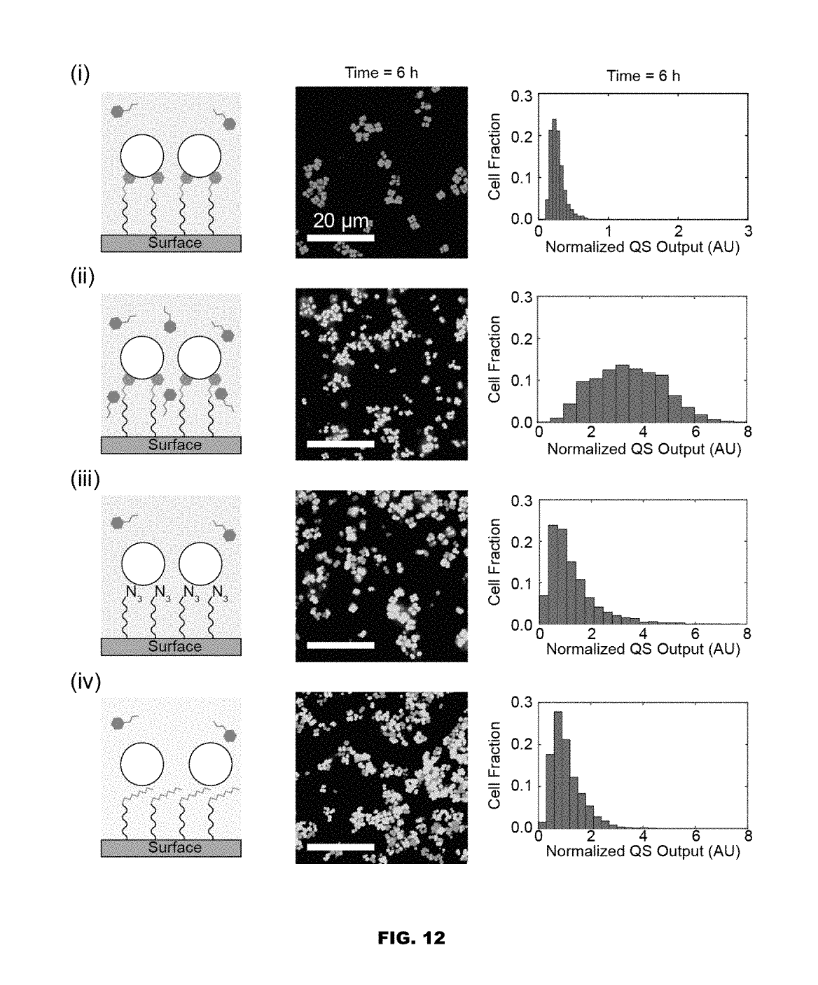

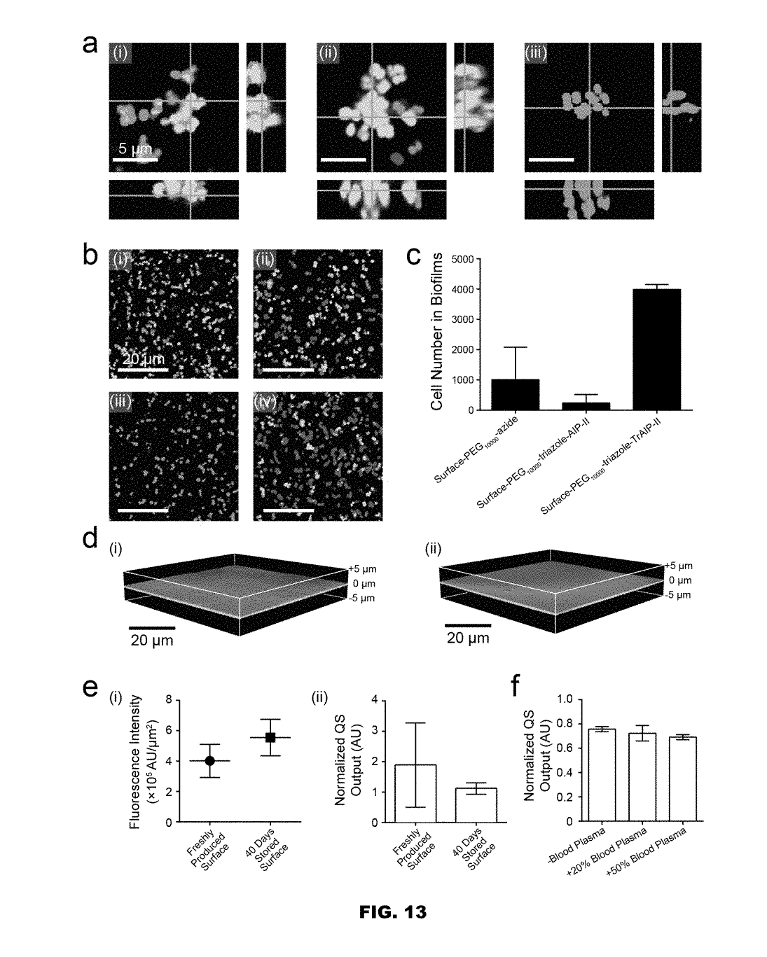

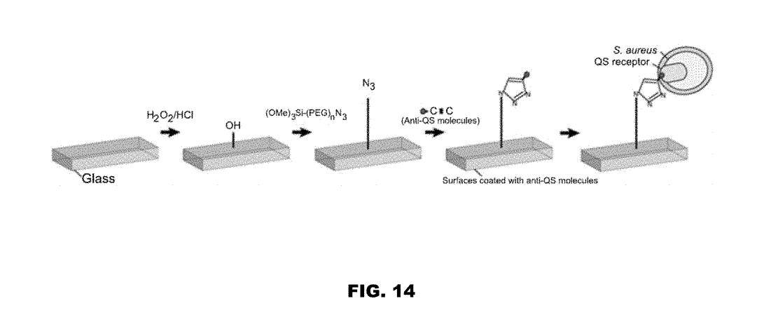

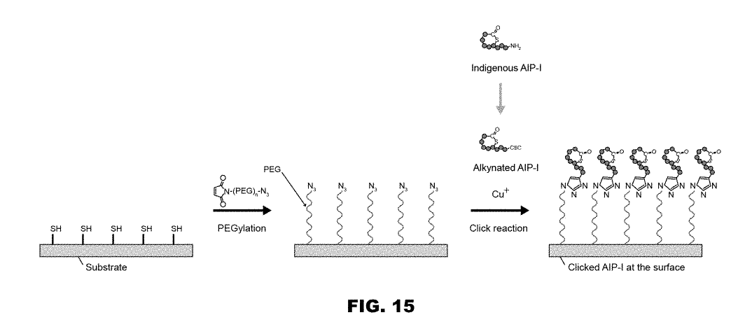

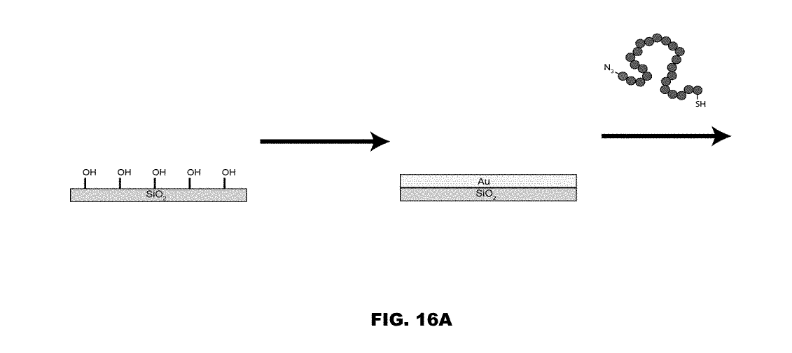

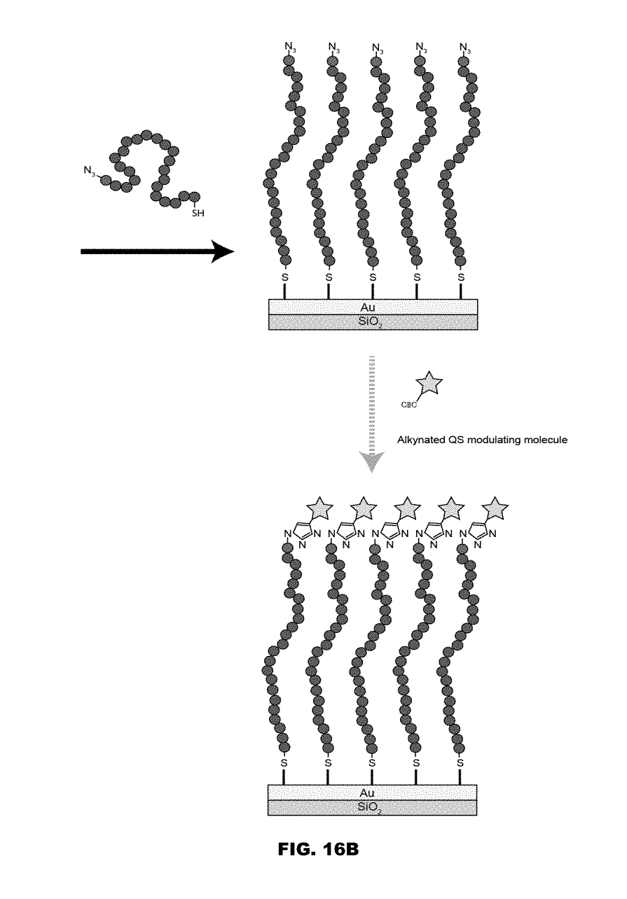

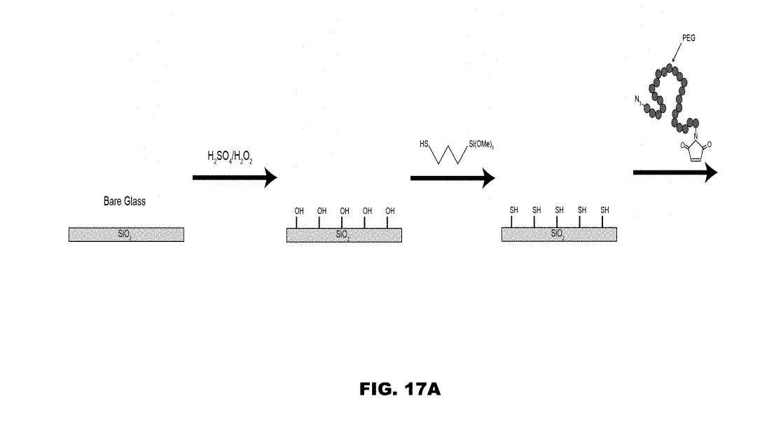

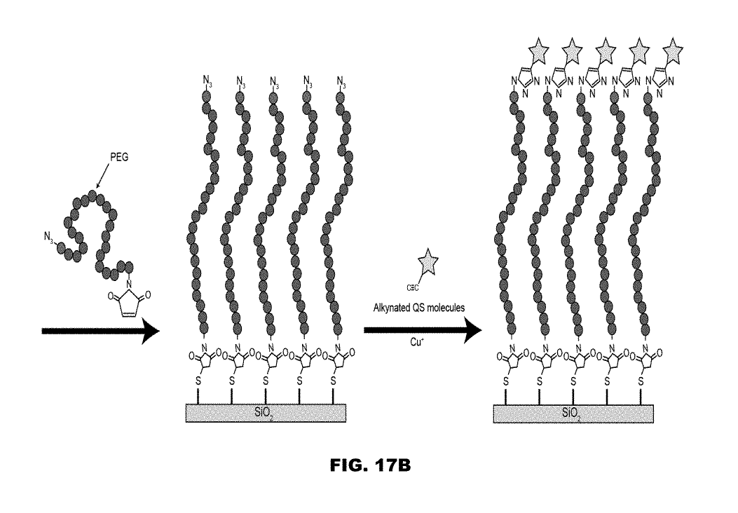

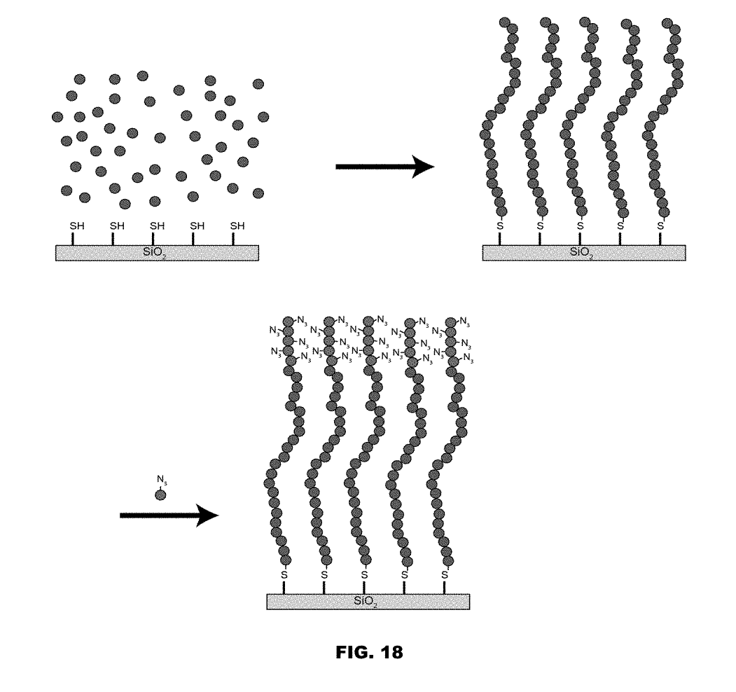

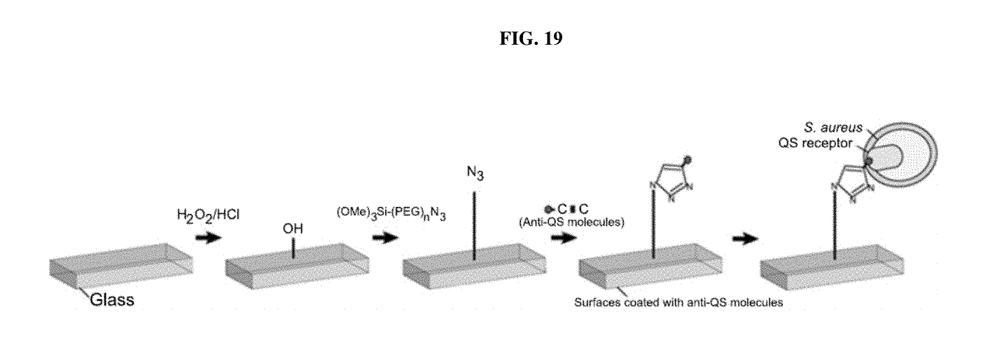

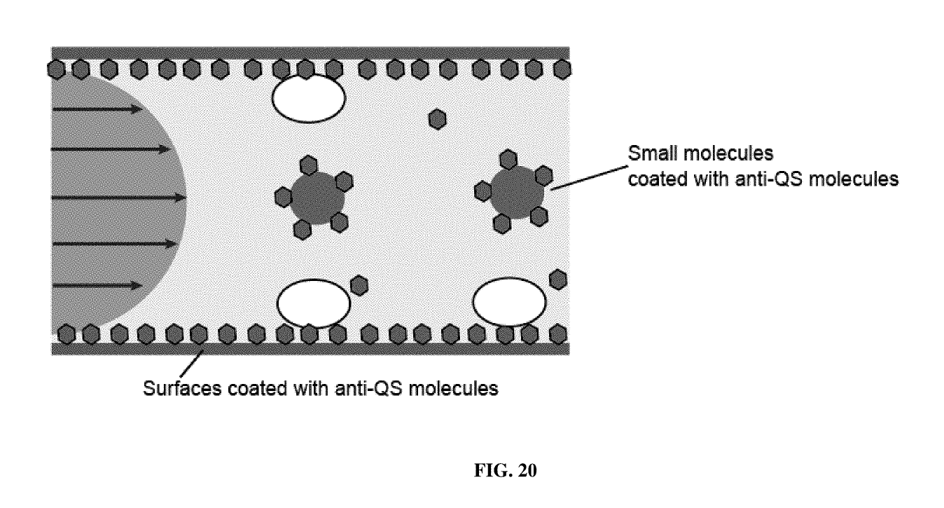

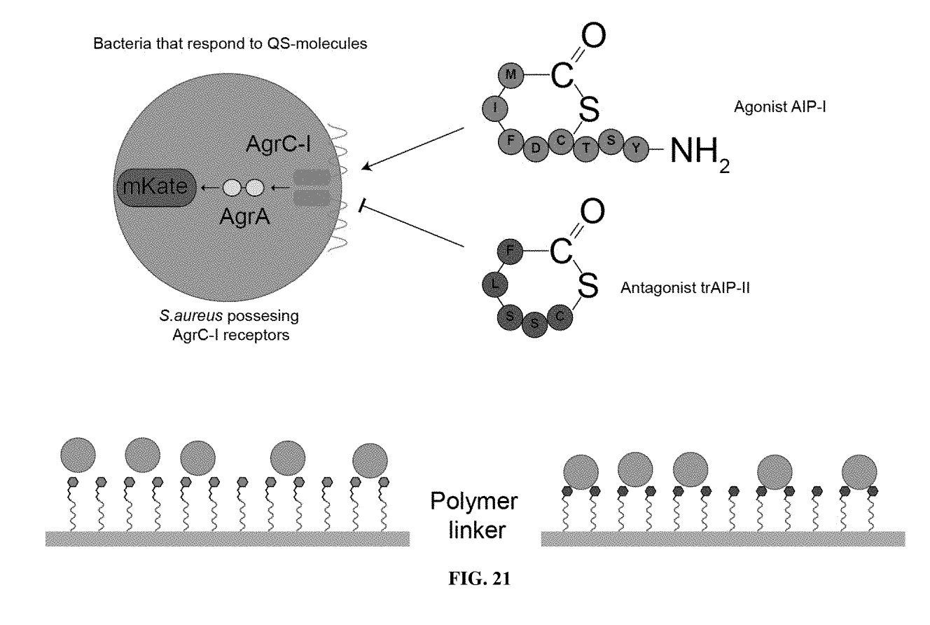

To make the AIP-I amenable to surface attachment, we synthesized AIP-I containing an alkyne at the N-terminus (FIG. 7a and FIG. 8a). We call this compound “Alkyne-AIP-I” (FIG. 2a). Prior to attaching the Alkyne-AIP-I to the surface linkers, we carried out the click reaction between the Alkyne-AIP-I and a surface-free version of the PEG₃₃₀-azide linker to test if the AIP-I derivatives retained their activation capability following the click reaction. The reaction between the alkyne and azide produces a triazole ring linking AIP-I to the PEG₃₃₀ polymer. We call this compound “PEG₃₃₀-triazole-AIP-I” (FIG. 2a (iii), FIG. 7a and FIG. 8b). In solution, AIP-I, Alkyne-AIP-I, and PEG₃₃₀-triazole-AIP-I activated Agr QS in the S. aureus reporter strain with similar efficacy albeit with different potencies (FIG. 2a (iii) and FIG. 2b). The calculated EC50 values were 28 nM (±3) for AIP-I, 190 nM (±40) for Alkyne-AIP-I, and 1.1 μM (±0.20) for PEG₃₃₀-triazole-AIP-I (Table 6). Addition of 2.5 μM TrAIP-II repressed the Agr QS response to all three compounds by a comparable magnitude (FIG. 2b). We interpret these results to mean that, analogous to AIP-I, both Alkyne-AIP-I and the PEG₃₃₀-triazole-AIP-I activate QS by targeting the AgrC-I receptor.

For the surface modification work, we used silanization and maleimide-thiol chemistry to decorate the surface with PEG₁₀₀₀₀ polymers carrying azide moieties at one end (FIG. 9). We name this unit the “Surface-PEG₁₀₀₀₀-azide”. Calculations of the height of the PEG brush suggest that a PEG₁₀₀₀₀ polymer would have sufficient length to span the peptidoglycan layer and position the attached AIP-I to interact with the AgrC-I receptor located on the cell membrane. We could not, however, carry out our companion solution assay with the PEG₁₀₀₀₀ polymer attached to AIP-I due to limitations in purification and characterization of soluble PEG₁₀₀₀₀ entities. In solution, PEG₁₀₀₀₀ polymers adopt a random-coiled conformation that does not mimic the extended conformation they possess when they are attached to surfaces with high grafting density. We carried out the click reaction to covalently attach the Alkyne-AIP-I to the Surface-PEG₁₀₀₀₀-azide (FIG. 2a (iv)), generating “Surface-PEG₁₀₀₀₀-triazole-AIP-I”. To examine whether the PEG-triazole-AIP-I moiety remained functional when attached to the surface, we provided the S. aureus reporter strain to the Surface-PEG₁₀₀₀₀-triazole-AIP-I in microfluidic chambers. The Agr QS response was induced (see “Surface Assay”, FIG. 2a (iv) and FIG. 2c). Compared to T=0 h when cells were in the QS-off mode, the response was activated over time and reached an average of 25-fold activation at T=6 h (FIGS. 2c, 2d and 10a). These results suggest that the surface-attached AIP-I is indeed recognized as an autoinducer by the cognate membrane-bound AgrC-I receptor. To confirm our interpretation of surface-tethered AIP-I eliciting the Agr QS response in S. aureus, 2.5 □ M TrAIP-II antagonist was provided in solution to the S. aureus cells residing on the AIP-I coated surface. The Agr QS response was repressed (FIG. 2c and FIG. 10b (i)).

To reinforce the above results, we show that S. aureus did not activate a QS response when introduced onto the identical surface lacking the triazole-AIP-I decoration (Surface-PEG₁₀₀₀₀) or that had not undergone the click reaction (Surface-PEG₁₀₀₀₀-azide) (FIG. 2c, FIG. 10b (ii) and FIG. 10b (iii)). Furthermore, the reporter strain that was presented a surface coated with the identical PEG₁₀₀₀₀ polymer attached, via a triazol ring, to a ring-opened version of AIP-I did not elicit a response (see compound 6 in FIG. 7a, FIG. 2c and FIG. 10b (iv); called Surface-PEG₁₀₀₀₀-triazole-Linear-AIP-I). Identical results were obtained using the Surface-PEG₁₀₀₀₀-triazole-AIP-I in which the thioester ring was opened via treatment with 10 mM cysteine (FIG. 10b (v)). Consistent with these results, an S. aureus reporter strain lacking AgrC-I did not respond to the surface-attached AIP-I (FIG. 10b (vi)). Thus, surface-attached AIP-I specifically and reversibly binds to AgrC-I , indicating that it functions as an autoinducer.

FIG. 3 illustrates a surface-attached Agr QS antagonist, TrAIP-II, that inhibits the S. aureus QS response to AIP-I. FIG. 3a. Strategy for modification of TrAIP-II and attachment to the surface; (i) TrAIP-II, (ii) Alkyne-TrAIP-II, (iii) PEG₃₃₀-triazole-TrAIP-II, and (iv) Surface-PEG₁₀₀₀₀-triazole-TrAIP-II. FIG. 3b. The S. aureus Agr QS reporter assay in solution with 100 nM AIP-I (asterisks), TrAIP-II+100 nM AIP-I (circles), Alkyne-TrAIP-II+100 nM AIP-I (squares), PEG₃₃₀-triazole-TrAIP-II+100 nM AIP-I (triangles), and PEG₃₃₀-triazole-TrAIP-II (diamonds) is shown. Normalized Agr QS outputs were measured as a function of the concentrations of TrAIP-II and its derivatives. PEG₃₃₀-triazole-TrAIP-II (diamonds) was measured at only the two highest concentrations due to limitations in available quantities of the reagent. Data points indicate means, and error bars denote standard deviations from n=4 experiments. FIG. 3c. The Agr QS surface inhibition assay was performed with 30 nM AIP-I in solution and the following surfaces: Surface-PEG₁₀₀₀₀-triazole-TrAIP-II (asterisks), Surface-PEG₁₀₀₀₀-azide (circles), and Surface-PEG₁₀₀₀₀-triazole-Linear-TrAIP-II (squares). 1 μM AIP-I was added in solution with the Surface-PEG₁₀₀₀₀-triazole-TrAIP-II (diamonds). The normalized Agr QS outputs were measured for 1000-4000 individual cells in each experiment. Data points indicate means, and error bars denote standard deviations from triplicate experiments. An ANOVA test with Tukey-Kramer post hoc analysis was used to assess the statistical significance between the means for the TrAIP-II coated surface and the control surfaces using the T=6 h data (**** P<0.0001). FIG. 3d. Merged fluorescence images of the S. aureus reporter strain on the Surface-PEG₁₀₀₀₀-triazole-TrAIP-II+30 nM AIP-I in bulk at T=0, 2, 4, and 6 h are shown. Images are based on n=3 independent experiments; one representative image for each condition was chosen from ˜50 images acquired from different regions of each surface. Scale bars: 20 μm.

FIG. 4 illustrates that wild-type S. aureus responded to surface-attached AIP-I and TrAIP-II. FIG. 4a. Normalized Agr QS outputs were measured for Surface-PEG₁₀₀₀₀-azide (black circles), Surface-PEG₁₀₀₀₀-triazole-AIP-I (blue triangles), and Surface-PEG₁₀₀₀₀-triazole-TrAIP-II (pink diamonds). The split pink-blue square symbols show the result when 1 μM AIP-I was supplied in solution in the Surface-PEG₁₀₀₀₀-triazole-TrAIP-II experiments. In each experiment, normalized QS outputs were measured as means from ˜2000 individual cells. Data points indicate means, and error bars denote standard deviations from triplicate experiments. An ANOVA test with Tukey-Kramer post hoc analysis was used to assess the statistical significance between the means from the Surface-PEG₁₀₀₀₀-azide and the QS molecule coated surfaces using the T=6 h data (**** P<0.0001). FIG. 4b. Merged fluorescent images of wild-type S. aureus agr-I(RN6390b), on modified surfaces at T=6 h are shown, see FIG. 6 for details (green: constitutive fluorescence, red: QS-controlled fluorescence). (i) Surface-PEG₁₀₀₀₀-azide, (ii) Surface-PEG₁₀₀₀₀-triazole-AIP-I, (iii) Surface-PEG₁₀₀₀₀-triazole-TrAIP-II, and (iv) Surface-PEG₁₀₀₀₀-triazole-TrAIP-II+1 μM AIP-I in bulk. Images are based on n=3 independent experiments; one representative image for each condition was chosen from ˜50 images taken from different regions of each surface. Scale bars: 20 μm. FIG. 4c. Three-dimensional renderings of biofilms (225×225×44 μm³) of the S. aureus agr-I strain (RN6390b) constitutively expressing mKO are shown. Biofilms were grown for 18 h on the following modified surfaces: (i) Surface-PEG₁₀₀₀₀-azide, (ii) Surface-PEG₁₀₀₀₀-triazole-AIP-I, and (iii) Surface-PEG₁₀₀₀₀-triazole-TrAIP-II. Images are based on n=3 independent experiments with 6 regions imaged in each replicate; one representative image for each condition was chosen from the 18 total images for each surface type. Scale bars: 50 μm. FIG. 4d. Number of S. aureus agr-I (strain RN6390b) cells in biofilms on the designated modified surfaces (225×225×44 μm³) at T=18 h (8 h under flow and 10 h with no flow, see examples, e.g., for protocol details). Data points indicate means, and error bars denote standard deviations from triplicate experiments.

FIG. 5 illustrates the features of autoinducer-attached surfaces. FIG. 5a. Stability of surface-attached molecules. Mean surface fluorescence intensities were measured from the Surface-PEG₁₀₀₀₀-triazole-dye over time in the presence and absence of S. aureus cells. Alexa Fluor 555 alkyne dye (Thermo Fisher, Mass.) was attached to the surface using the click reaction. Data points indicate means, and error bars denote standard deviations from n=4 experiments. FIG. 5b. Stability of surface-attached AIP-I and its activity following repeated introduction of S. aureus cells. (i) The relevant genotypes and fluorescent reporters used for the multiple inoculations; mtur2 denotes mturquoise2. (ii) Representative merged fluorescence images of the S. aureus reporter strains on the Surface-PEG₁₀₀₀₀-triazole-AIP-I. At T=0 h, the S. aureus reporter strain (QS-off: blue, QS-on: purple) was introduced. At T=3 h, S. aureus cells were washed off the surface and the second reporter strain (QS-off: green, QS-on: yellow) was introduced to the same surface. Scale bar: 20 μm. (iii) Merged fluorescence images of the area colonized by both strains. The constitutive fluorescent colors from the two S. aureus reporter strains (blue, T=3 h and green, post-wash, T=3 h) were artificially aligned. The light blue area with the white circles shows that cells from both the first and second inoculations colonized the same region. In panel b (ii) and b (iii), images are based on n=3 independent experiments; one representative image for each condition was chosen from ˜10 images taken from different regions of each surface. (iv) Normalized Agr QS outputs were measured at 3 h after each strain was introduced onto the same surface. Data points indicate means, and error bars denote standard deviations from triplicate experiments. FIG. 5c. Mixtures of dye molecules containing alkyne moieties can be simultaneously attached to the Surface-PEG₁₀₀₀₀-azide. Dyes: Alexa Fluor 488 alkyne (green) and Alexa Fluor 594 alkyne (red). Three-dimensional renderings of the surface that underwent the click reaction with a mixture of the two dyes are shown. Images are based on n=4 independent experiments.

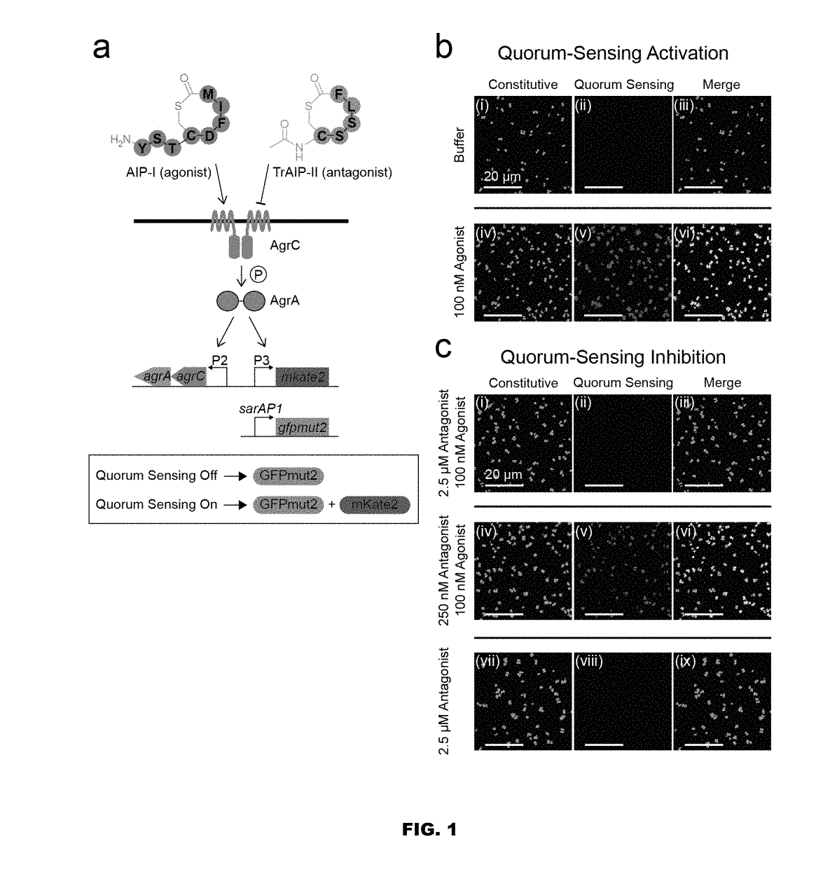

FIG. 6. S. aureus exhibits heterogeneous QS responses. Single cell-level analyses of the data in FIG. 6a showed that the S. aureus reporter cells displayed heterogeneous QS responses (also shown in FIG. 1b (vi) and FIG. 1c (vi)). To identify the source of heterogeneity, the fluorescent protein distributions in individual cells was measured under different conditions. The reporter cells that were used harbored the Agr-activated agrP3 promoter fused to mkate2 (agrP3-mkate2) and carried plasmid-expressed agrCA encoding the AIP-I detection-response apparatus under its native promoter, agrP2 (agrP2-agrCA). Addition of 100 nM AIP-I to this strain resulted in a broad, skewed QS controlled mKate2 protein production profile (red) compared to the narrow distribution of the GFPmut2 protein produced from the constitutive gfpmut2 reporter gene (green) (FIG. 6a). The coefficient of variation for the normalized QS response was σ/μ=0.69 (±0.03) (yellow histogram in FIG. 6a). The broad mKate2 protein distribution could stem from differences in plasmid copy number per cell, variations in transcription from agrP3 and/or agrP2, variations in translation of the mKate2 protein and/or AgrC-I/AgrA, variations in agrP2-agrCA, agrP3-mkate2 gene copy numbers due to plasmid expression, or from intrinsically noisy S. aureus QS responses due to feedback. Each of these possibilities was examined as follows.

First, it was tested whether heterogeneity arises from variation in mKate2 fluorescent protein translation or from plasmid copy number effects. To do this, the agrP3 promoter driving mkate2 was replaced with the constitutive sarAP1 promoter on the plasmid (sarAP1-mkate2). The corresponding histogram depicting the normalized mKate2 fluorescence is narrower when sarAP1, rather than agrP3, is the promoter upstream of mkate2 (σ/μ=0.22 (±0.02), yellow histogram in FIG. 6b). This result makes two points: first, mKate2 fluorescent protein translation is not responsible for the heterogeneity that was observed. Second, the result in FIG. 6b suggests that fluorescent protein production from the constitutive sarAP1 promoter is homogeneous, irrespective of whether the gene is located on a plasmid or on the genome. Thus, plasmid copy number effects do not cause heterogeneity in this reporter system.

Next, it was tested whether heterogeneity arises from variation in copy numbers of genes encoding the QS-detection/response apparatus (agrP2-agrCA) from the plasmid. For this test, the agrP2-agrCA genes were introduced into the genome rather than having them expressed from a plasmid. The reporter gene, agrP3-mkate2, was expressed from the plasmid (FIG. 6c). Addition of 100 nM AIP-I to this strain resulted in less broad, less skewed QS controlled mKate2 protein production (red) compared to that of FIG. 6a. The value of σ/μ for the normalized fluorescence distribution from the strain used in FIG. 6c could not be determined, because the strain did not possess the constitutively expressed fluorescent protein gene. In a separate experiment, wild-type S. aureus RN6930b that encodes agrP2-agrCA was used as well as the agrBD genes on the genome, which provides the strain with the capacity to make endogenous AIP-I (FIG. 6d). This wild-type strain also harbored the constitutively expressed sarAP1-mkate2 gene as well as the QS-controlled reporter gene, agrP3-gfpmut2 on the same plasmid (FIG. 6d). This strain exhibited normalized fluorescence with σ/μ=0.58 (±0.10), which is broader than that from the strain shown in FIG. 6b. These results were interpreted to mean that expression of the agrCA genes from a plasmid that exists at about ˜20 copies per cell^{S8} contributed to the observed heterogeneity but cannot be the sole source responsible for heterogeneity.

Lastly, it was tested whether heterogeneity arises from variation in copy number of the agrP3-mkate2 gene. For this assessment, both the agrP3-mkate2 gene and the agrP2-agrCA gene were introduced into the genome (FIG. 6e). This arrangement differed from the reporter strain shown in FIG. 6a, which contained both agrP2-agrCA and agrP3-mkate2 on the plasmid. Addition of 100 nM AIP-I to these cells resulted in a distribution with σ/μ=0.20 (±0.04) that was narrower than that from the strains shown in FIGS. 6a, 6c, and 6d. This result suggested that expression of the agrP3-mkate2 gene from a multi-copy plasmid also partially contributed to the observed heterogeneity.

Taken together, these data indicate that heterogeneity from the reporter strain (FIG. 6a) is a consequence of expression of the agrP3-mkate2 gene and the agrP2-agrCA gene when present on multi-copy plasmids. One possibility is that the cells possess different numbers of AgrC-I receptors on their surfaces and this results in variation. AIP-I stimulation is known to induce a positive autoinduction feedback loop that promotes increased expression of agrC^{S9}. Initial differences in AgrC-I receptor numbers could be amplified through this feedback. This hypothesis is supported by our observation that the measured variation positively depended on the concentration of AIP-I provided to the cells. Positive feedback could increase noise and make the agrP3-mkate2 distribution broad, and such noise could be further amplified when there exist multiple copies of the agrP3-mkate2 and the agrP2-agrCA genes when present on a plasmid (FIG. 6a). With respect to the present work, these analyses show that, while the plasmid-mediated agrP3-mkate2 response to AIP-I is broad, it is a reliable and amplified readout of QS in the S. aureus reporter strain (FIG. 6a).

Each panel is discussed in more detail as follows: FIG. 6a. The reporter strain used was S. aureus ΔagrBDCA carrying a constitutively expressed sarAP1-gfpmut2 transcriptional fusion in the genome. The strain also harbored the QS-controlled agrP3-mkate2 reporter and the agrCA autoinducer-detection/response genes on a plasmid under their native agrP2 promoter. The agrP3-mkate2 and agrP2-agrCA genes were cloned in the opposite orientation. FIG. 6b. The S. aureus AagrBDCA strain with sarAP1-gfpmut2 in the genome and sarAP1-mkate2 on a plasmid. FIG. 6c. The S. aureus AagrBD strain carrying agrP2-agrCA in the chromosome. The agrP3-mkate2 fusion was carried on a plasmid. This strain did not possess the constitutive reporter gene. FIG. 6d. Wild-type S. aureus carrying sarAP1-mkate2 and agrP3-gfpmut2 on the same plasmid. FIG. 6e. The S. aureus ΔagrBD strain carrying agrP3-mkate2 and agrP2-agrCA in the chromosome. The sarAP1-gfpmut2 fusion was carried on a plasmid. In panel FIG. 6d, the fluorescent colors were reversed to make them consistent with the reporter colors shown in panel FIG. 6a. The identical color code was maintained in all panels for ease of comparison. The top panels show the relevant genotypes, fluorescent reporters, and an image of the fluorescent cells. Below the micrograph images, in each vertical row, are representative histograms of the single-cell expression from the constitutive promoter (top) and the QS-controlled promoter (middle) from populations containing thousands of cells. Images were based on n=3 independent experiments for each condition. The bottom panels in FIG. 6a, FIG. 6d and FIG. 6e show histograms of the normalized fluorescence (yellow), which was defined as the QS-controlled reporter output divided by the output from the constitutive reporter in individual cells. The bottom panel in FIG. 6b shows a histogram of the normalized fluorescence (yellow), which was defined as the constitutive red fluorescent reporter output divided by the output from the constitutive green fluorescent reporter in individual cells. FIG. 6a, FIG. 6c, FIG. 6e,+100 nM AIP-I (agonist). FIG. 6b, FIG. 6d,+buffer. Scale bars: 20 μm.

FIG. 7. Synthesis scheme for a. AIP-I derivatives and b. TrAIP-II derivatives. Peptides were generated using Fmoc-based solid-phase peptide synthesis (SPPS) on hydrazine derivatized resins followed by cleavage with trifluoroacetic acid (TFA). Hydrazide peptides (1) and (7) were oxidized with NaNO₂ and subsequently underwent MESNa (sodium 2-sulfanylethanesulfonate) thiolysis. Thioesters (2) and (8) were purified with reverse phase-high-performance liquid chromatography (RP-HPLC), and then treated with TCEP (3,3′,3″-Phosphanetriyltripropanoic acid) to remove the −StBu protecting group, and cyclized in buffer at pH=7, generating, via intermediates (3) and (9), compounds (4) and (10), which correspond to the Alkyne-AIP-I and Alkyne-TrAIP-II, respectively, referenced herein. The Alkyne-AIP-II was synthesized using an identical method. PEG₃₃₀-triazole-AIP-I (5) and PEG₃₃₀-triazole-TrAIP-II (11) were produced via the copper (I) catalyzed alkyne-azide cycloaddition (CuAAC) click reaction with O-(2-Azidoethyl) heptaethylene glycol. Alkyne-Linear-AIPs (6) and (12) were produced by removing -StBu from (1) and (7).

FIG. 8. Characterization of purified AIP-I and TrAIP-II derivatives by RP-HPLC (left) and electrospray ionization-mass spectrometry (ESI-MS, right). FIG. 8a. Alkyne-AIP-I. Expected [M+H]⁺=1041.4 Da. Observed [M+H]⁻⁼1041.4 Da. FIG. 8b. PEG₃₃₀-triazole-AIP-I. Expected [M+H]²⁺/2=718 Da. Observed [M+H]²⁺/2=718.7 Da. FIG. 8c. Alkyne-TrAIP-II. Expected [M+H]⁺=618.3 Da. Observed [M+H]⁺⁼618.2 Da. FIG. 8d. PEG₃₃₀-triazole-TrAIP-II. Expected [M+H]⁺=1013.4 Da. Observed [M+H]⁺=1013.4 Da. Red boxed peaks are shown magnified at the right of each panel. Double asterisks (**) denote [M+2H]²⁺/2.

FIG. 9. Chemical modifications of surfaces and surface chemistry characterization. FIG. 9a. Schematic of the reactions to generate the Surface-PEG₁₀₀₀₀-triazole-AIP-I. The glass surface was hydroxylated using the piranha reaction, followed by silanization to attach thiol (-SH) moieties. Next, the thiol-decorated surface was linked with a PEG₁₀₀₀₀ polymer functionalized with a maleimide moiety at one terminus, using an addition reaction. The PEG polymer linker also harbored an azide at the other terminus. This azide was subjected to a click reaction to attach molecules carrying alkyne groups, such as the Alkyne-AIP-I. FIG. 9b. Attenuated total reflectance infrared spectra of the Surface-PEG₁₀₀₀₀-azide. Peaks at 970-1250 cm⁻¹ showed strong signals from C-O bonds, and peaks at 2850-3000 cm⁻¹ showed weak signals from C—H bonds in methylene moieties. These results indicated that the PEG polymer was attached to the surface. The azido moiety had a relatively low abundance peak due to the N_{3/}C—H ratio per tether being ˜1/880, which is below the detection limit. FIG. 9c. The Alexa Fluor 555 dye functionalized with an alkyne moiety (Thermo Fisher, MA) was used to characterize the surfaces. (i) Surface-PEG_{10000.} (ii) Surface-PEG₁₀₀₀₀-azide. Left, Schematic of the surface, which was treated with the Alexa Fluor 555 alkyne dye (depicted by yellow stars) and underwent the click reaction. Right, Three-dimensional rendering of each surface following the click reaction (51×51×10 μm³). These results demonstrated that the Surface-PEG₁₀₀₀₀-azide was successfully conjugated to the dye while the Surface-PEG₁₀₀₀₀ was incapable of undergoing the click reaction to attach the dye to the surface. Image (i) and (ii) are based on n=5 and n=8 independent experiments, respectively. Scale bars: 20 μm.

FIG. 10. S. aureus Agr QS activated by surface-attached AIP-I. The S. aureus reporter strain was used to follow the Agr QS status for cells growing on non-modified and chemically-modified surfaces with confocal microscopy. Single-cell image analysis demonstrated that the S. aureus reporter cells exhibited a broad and skewed QS output growing on the Surface-PEG₁₀₀₀₀-triazole-AIP-I with σ/μ=0.67 (±0.03) (FIG. 10a), which was analogous to its response to native AIP-I in solution in FIG. 6a. 10a. (i) Schematic of the experimental strategy showing the Surface-PEG₁₀₀₀₀-triazole-AIP-I and the addition of S. aureus cells (depicted as white circles) in liquid. (ii) Distribution of constitutive output (left), QS-controlled output (middle), and the normalized QS-controlled output (right) from single-cell analyses at T=0, 2, 6 h. The magnified graph in the inset shows the normalized QS output at T=6 h taken from the left panel and plotted for the range of 0 to 0.1. FIG. 10b. Schematics (left), merged images at T=6 h (middle), and normalized QS outputs at T=6 h from single-cell analyses (right) of control experiments characterizing the different surface chemistries from FIG. 2c. (i) Surface-PEG₁₀₀₀₀-triazole-AIP-I+2.5 μM TrAIP-II in solution, (ii) Surface-PEG₁₀₀₀₀, which were exposed to the click solution containing the Alkyne-AIP-I for 3 h and rinsed with 50% dimethyl sulfoxide in water. (iii) Surface-PEG₁₀₀₀₀-azide, and (iv) Surface-PEG₁₀₀₀₀-triazole-Linear-AIP-I were generated via the click reaction between the Surface-PEG₁₀₀₀₀-azide and compound 6 in FIG. 7a. (v) Surface-PEG₁₀₀₀₀-triazole-AIP-I was produced as described and, subsequently, the thioester ring was opened by treatment with 10 mM cysteine (depicted as red scissors in the schematic) in 100 mM phosphate buffer (pH=7) for 1 h. (vi) Surface-PEG₁₀₀₀₀-triazole-AIP-I was seeded with S. aureus cells that lack the AgrC receptor (the designation AgrC⁻ is shown above the white circles depicting the cells). The strain was called MK245. All images shown in FIG. 10 are based on n=3 independent experiments for each condition; one representative image for each condition was chosen from ˜50 images acquired from different regions of each surface. Scale bars: 20 μm.

FIG. 11. The requirements for Agr QS activation by surface-attached AIP-I. 11a. Alexa Fluor 555 alkyne dye (Thermo Fisher, Mass.) was used to detect azide functional groups on coated surfaces. Three-dimensional rendering of each surface following the click reaction (51×51×10 μm³). (i) Surface-PEG₄₀₀ and (ii) Surface-PEG₄₀₀-azide. Images were based on n=3 independent experiments. Only the Surface-PEG₄₀₀-azide successfully underwent the click reaction to generate the Surface-PEG₄₀₀-triazole-dye. FIG. 11b. The S. aureus Agr QS output in response to the Surface-PEG₁₀₀₀₀-triazole-AIP-I (black circles), Surface-PEG₄₀₀-triazole-AIP-I (black triangles), and Surface-PEG₄₀₀-triazole-AIP-I+50 nM AIP-I in solution (blue triangles). Data points indicate means, and error bars denote standard deviations from triplicate experiments. 11c. Merged images at T=6 h. (i) Surface-PEG₁₀₀₀₀-triazole-AIP-I. (ii) Surface-PEG₄₀₀-triazole-AIP-I. (iii) Surface-PEG₄₀₀-triazole-AIP-I+50 nM AIP-I in solution. Images were based on n=3 independent experiments for each condition; one representative image for each condition was chosen from ˜50 images acquired from different regions of each surface. Scale bars: 20 μm. 11d. Single-molecule analysis to quantify surface coverage density. (i) A representative image was shown of individually discernable fluorescent spots on the surface, which were photobleached. Images were based on n=3 independent experiments. The Surface-PEG₁₀₀₀₀-azide had reacted with 0.1−10 nM Alexa Fluor 555 alkyne fluorophore. Using a peak-finding algorithm, background-subtracted intensities of each spot were measured over time (red circles in the image). Scale bar: 10 μm. (ii) Two representative time courses of fluorescence emission for a fluorophore-labeled spot, displaying stepwise decreases in intensity. Most spots decreased in intensity in one step following bleaching (top), and a small number of spots bleached in two steps (bottom). (iii) Fitting the bleaching step sizes for over 3,600 spots with a normal distribution, provided the average single-molecule intensity (7,000 (AU)). This value was used to quantify surface coverage density of the Surface-PEG₁₀₀₀₀-azide that had reacted with 100 μM Alexa Fluor 555 alkyne fluorophore (a typical click reaction concentration used in this study). 11e. The coverage density of surface-attached AIP-I affected the S. aureus Agr QS output. The surface coverage density of active AIP-I was reduced by mixing Alkyne-AIP-I and Alkyne-Linear-AIP-I at different ratios prior to attachment to the surface while maintaining the total concentration of substrate for attachment constant at 100 μM. The zero-coverage density of surface-attached AIP-I denoted the surface containing only the Linear-AIP-I. The normalized Agr QS outputs were measured 3 h after the S. aureus reporter strain was introduced onto the surfaces with different coverage densities. Data points indicate means, and error bars denote upper and lower limits from two independent experiments.

FIG. 12. S. aureus Agr QS was inhibited by surface-attached TrAIP-II. (i-iv) Schematics (left), merged images at T=6 h (middle), and normalized QS outputs at T=6 h from single-cell analyses (right). (i) Surface-PEG₁₀₀₀₀-triazole-TrAIP-II+30 nM AIP-I in solution, (ii) Surface-PEG₁₀₀₀₀-triazole-TrAIP-II+1 μM AIP-I in solution, (iii) Surface-PEG₁₀₀₀₀-azide+30 nM AIP-I in solution, and (iv) Surface-PEG₁₀₀₀₀-triazole-Linear-TrAIP-II+30 nM AIP-I in solution. Surface-PEG₁₀₀₀₀-triazole-Linear-TrAIP-II was produced via the click reaction between the Surface-PEG₁₀₀₀₀-azide and compound 12 in FIG. 7b. Images are based on n=3 independent experiments for each condition; one representative image for each condition was chosen from ˜50 images acquired from different regions of each surface. Scale bars: 20 μm.

FIG. 13. QS response of wild-type S. aureus to surface-attached AIP-I, AIP-II, and TrAIP-II, chemical modification of metal and plastic surfaces, and examination of stability of the chemically-modified surfaces. FIG. 13a. Centered merged images showing single optical sections of the x-y plane at 2μm above the interface of the surface and the cell cluster, with z projections shown to the right (x-z plane) and below (y-z plane). Wild-type S. aureus agr-I (RN6390b) was grown for 3 h after inoculation on (i) Surface-PEG₁₀₀₀₀-azide, (ii) Surface-PEG₁₀₀₀₀-triazole-AIP-I, and (iii) Surface-PEG₁₀₀₀₀-triazole-TrAIP-II. Note: The fluorescent colors for wild-type S. aureus agr-I (RN6390b) were reversed to make them consistent with the colors of the reporter strains used elsewhere; green: constitutive fluorescence, red: QS-controlled fluorescence. Images are based on n=3 independent experiments for each condition; one representative image for each condition was chosen from ˜50 images acquired from different regions of each surface. FIG. 13b. Merged fluorescent images of S. aureus MRSA agr-I cells grown on the modified surfaces at T=6 h. Note: The fluorescent colors for the S. aureus MRSA cells were reversed to make them consistent with the colors of the reporter strains used elsewhere; green: constitutive fluorescence, red: QS-controlled fluorescence. (i) Surface-PEG₁₀₀₀₀-azide, (ii) Surface-PEG₁₀₀₀₀-triazole-AIP-I, (iii) Surface-PEG₁₀₀₀₀-triazole-TrAIP-II, and (iv) Surface-PEG₁₀₀₀₀-triazole-TrAIP-II+1 μM AIP-I in bulk. Images were based on n=2 independent experiments for each condition; one representative image for each condition was chosen from ˜50 images acquired from different regions of each surface. Scale bars: 20 μm. 13c. Number of S. aureus agr-II cells (strain RN6607) in biofilms grown for 18 h on the designated modified surfaces (225×225×44 μm³). Data points indicate means, and error bars denote standard deviations from triplicate experiments. FIG. 13d. The Alexa Fluor 555 dye functionalized with an alkyne moiety (Thermo Fisher, MA) was used to characterize coating of gold and PDMS surfaces. Three-dimensional rendering of each surface following the click reaction (51×51×10 μm³). (i) Gold-based Surface-PEG5000-triazole-dye. (ii) PDMS-based Surface-PEG₁₀₀₀₀-triazole-dye. Images in panels (i) and (ii) were based on n=2 independent experiments. Chemical modifications of gold- and PDMS-coated surfaces are described herein. The results show that both metal and plastic coated surfaces were successfully conjugated to the dye using the click reaction. Scale bars: 20 μm. FIG. 13e. Investigation of stability of coated surfaces. (i) Mean surface fluorescence intensities were measured from freshly-prepared Surface-PEG₁₀₀₀₀-triazole-dye (circle) and the Surface-PEG₁₀₀₀₀-triazole-dye following 40 days of storage (square). Data points indicate means, and error bars denote standard deviations from triplicate experiments. (ii) The normalized Agr QS output was measured 3 h after the S. aureus agr-I reporter strain was introduced onto a freshly-prepared Surface-PEG₁₀₀₀₀-triazole-AIP-I (left) and onto an identical surface following 40 days of storage (right). Data points indicate means, and error bars denote standard deviations from triplicate experiments. FIG. 13f. The normalized Agr QS output was measured 3 h after the S. aureus agr-I reporter strain was introduced onto the Surface-PEG₁₀₀₀₀-triazole-AIP-I that was never exposed to blood plasma (left) or that had been pre-treated with 100% human blood plasma with a further 20% (middle) or 50% (right) blood plasma solution present throughout the experiment. Data points were acquired after 3 h and indicated means from two independent experiments. Error bars denote upper and lower limits.

FIG. 14 is an illustration of a process by which a QS modulating molecule can be attached to a surface via a linker, rendering the surface-attached molecule capable of binding to a QS receptor on a bacterial cell. As described herein, QS modulating molecules (e.g., an anti-QS molecule that binds to a QS receptor of a bacterial organism) was attached to a surface via a linker using this process. In this example, the glass slide underwent hydroxylation using acidic solution to form free hydroxyl groups on the glass surface. The hydroxylated surface was treated with a PEG-based linker functionalized with silane moieties at one terminus under conditions suitable for the linker to attach to the surface via silanization. The anti-QS molecule was then attached under suitable conditions to the linker.

FIG. 15 is an illustration of a process by which a QS modulating molecule can be attached to a surface through a PEG-based linker using thiol-ene reactions and click chemistry. First, a substrate comprising —SH— moieties was treated with a PEG-based linker functionalized with maleimide moieties at one terminus under conditions suitable for the linker to attach to the —SH— group on the surface. Then, the alkynated form of agonist AIP-I was attached to the linker using click chemistry. As described herein, this process was used to attach AIP-I to the surface via the PEG-based linker.

FIGS. 16A-16B are an illustration of a process by which a QS modulating molecule can be attached to a gold plated surface using gold-sulfide bond formation, a linker and click chemistry. As described herein, this process was used to attach a QS modulating molecule to a gold plated surface via a linker. First, a substrate comprising free hydroxyl moieties was coated with gold. Next, the gold surface was treated with a linker functionalized with thiol moieties at one terminus under conditions suitable for the linker to attach to the gold surface via a gold-sulfide bond formation. The alkynated QS modulating molecule was attached to the linker under suitable conditions, such as click chemistry. It is understood that this process begins at FIG. 16A and continues to FIG. 16B.

FIGS. 17A-17B are an illustration of a process by which a QS modulating molecule can be attached to a surface through a linker using thiol-ene reactions and click chemistry. As described herein, this process was used to attach a linker to a surface. In this example, the glass slide underwent hydroxylation to form hydroxyl groups on the glass surface. The hydroxylated surface was converted to a surface comprising —SH— moieties via silanization, and then treated with a PEG-based linker functionalized with maleimide moieties at one terminus, under conditions suitable for the linker to attach to the surface. An alkynated QS molecule was then attached to the linker (e.g., using click chemistry), rendering the surface-attached molecule capable of binding to a QS receptor on a bacterial cell. It is understood that this process begins at FIG. 17A and continues to FIG. 17B.

FIG. 18 is an illustration of a process by which a QS modulating molecule can be attached to a surface through a linker, wherein the linker is generated using surface-initiated polymerization RAFT and click chemistry. As described herein, this process was used to generate a linker on a surface at the —SH— sites via polymerization in situ, followed by attaching an alkynated QS molecule to the linker (e.g., using click chemistry).

FIG. 19 is another illustration of a process by which a QS modulating molecule can be attached to a surface via a linker, rendering the surface-attached molecule capable of binding to a QS receptor on a bacterial cell. As described herein, QS modulating molecules (e.g., an anti-QS molecule that binds to a QS receptor of a bacterial organism) was attached to a surface via a linker using this process. In this example, the glass slide underwent hydroxylation using acidic solution to form free hydroxyl groups on the glass surface. The hydroxylated surface was treated with a PEG-based linker functionalized with silane moieties at one terminus under conditions suitable for the linker to attach to the surface via silanization. The anti-QS molecule was then attached under suitable conditions to the linker.

FIG. 20 illustrates a QS modulating molecule (shown as a hexagon) coating any shaped surface, thereby altering QS, biofilm production, biofilm streamer production, and/or virulence factor production. In this figure, the black arrows represent fluid flow, the dark gray lines represent surfaces, the circles represent small molecules/beads coated with QS modulating molecules, and the white ovals represent bacterial cells.

FIG. 21 is an illustration of a process by which QS modulating molecules can be attached to a surface via a polymer linker. In this example, the agonist AIP-I/antagonist trAIP-II binds to the AgrC-I receptor to promote/inhibit QS in S. aureus. Bacteria have been shown to respond (via the AgrC-I receptor) to the QS-modulating molecules (either the agonist or antagonist) attached via the polymer linker to the surface.

### DETAILED DESCRIPTION

### A. Definitions

Unless defined otherwise, all technical and scientific terms used herein have the same meaning as commonly understood by those of ordinary skill in the art, such as in the arts of peptide chemistry, cell culture, nucleic acid chemistry, and biochemistry. Standard techniques are used for molecular biology, genetic and biochemical methods (see, Sambrook et al., Molecular Cloning: A Laboratory Manual, 3^{rd} ed., 2001, Cold Spring Harbor Laboratory Press, Cold Spring Harbor, N.Y.; Ausubel et al., Short Protocols in Molecular Biology (1999) 4^{th} ed., John Wiley & Sons, Inc.), which are incorporated herein by reference.

In the present invention, a “microorganism” is defined as a bacterium, archaeon, protozoan, fungus, and/or alga.

In the present invention, “bacteria” are defined as any one of a large domain of single-celled prokaryotic microorganisms. As used herein, bacteria include any that are known to those of ordinary skill in the art and any that may be discovered. Preferred examples of bacteria are those known to be pathogenic to humans, animals or plants. Other preferred examples include those known to cause undesirable contamination and/or clogging of industrial flow systems. Still other preferred examples of bacteria include those known to infect implanted medical devices (e.g., pumps, stents, artificial joints, screws, rods, and the like). Further preferred examples of bacteria include those capable of forming biofilms and/or biostreamers or producing virulence factors. Further preferred examples include bacteria selected from the following genera: Abiotrophia, Achromobacter, Acidaminococcus, Acidovorax, Acinetobacter, Actinobacillus, Actinobaculum, Actinomadura, Actinomyces, Aerococcus, Aeromonas, Afipia, Agrobacterium, Alcaligenes, Alloiococcus, Alteromonas, Amycolata, Amycolatopsis, Anabaena, Anabaenopsis, Anaerobospirillum, Anaerorhabdus, Aphanizomenon, Arachnia, Arcanobacterium, Arcobacter, Arthrobacter, Atopobium, Aureobacterium, Bacillus, Bacteroides, Balneatrix, Bartonella, Bergeyella, Bifidobacterium, Bilophila, Bordetella, Borrelia, Brachyspira, Branhamella, Brevibacillus, Brevibacterium, Brevundimonas, Brucella, Burkholderia, Buttiauxella, Butyrivibrio, Calymmatobacterium, Camesiphon, Campylobacter, Capnocytophaga, Capnylophaga, Cardiobacterium, Catonella, Cedecea, Cellulomonas, Centipeda, Chlamydia, Chlamydophila, Chromobacterium, Chryseomonas, Chyseobacterium, Citrobacter, Clostridium, Collinsella, Comamonas, Corynebacterium, Coxiella, Cryptobacterium, Cyanobacteria, Cylindrospermopsis, Delftia, Dermabacter, Dermatophilus, Desulfomonas, Desulfovibrio, Dialister, Dichelobacter, Dolosicoccus, Dolosigranulum, Edwardsiella, Eggerthella, Ehrlichia, Eikenella, Empedobacter, Enterobacter, Enterococcus, Erwinia, Erysipelothrix, Escherichia, Eubacterium, Ewingella, Exiguobacterium, Facklamia, Filifactor, Flavimonas, Flavobacterium, Francisella, Fusobacterium, Gardnerella, Gemella, Globicatella, Gloeobacter, Gordona, Haemophilus, Hafnia, Hapalosiphon, Helicobacter, Helococcus, Hemophilus, Holdemania, Ignavigranum, Johnsonella, Kingella, Klebsiella, Kocuria, Koserella, Kurthia, Kytococcus, Lactobacillus, Lactococcus, Lautropia, Leclercia, Legionella, Leminorella, Leptospira, Leptospirae, Leptotrichia, Leuconostoc, Listeria, Listonella, Lyngbya, Megasphaera, Methylobacterium, Microbacterium, Micrococcus, Microcystis, Mitsuokella, Mobiluncus, Moellerella, Moraxella, Morganella, Mycobacterium, Mycoplasma, Myroides, Neisseria, Nocardia, Nocardiopsis, Nodularia, Nostoc, Ochrobactrum, Oeskovia, Oligella, Orientia, Paenibacillus, Pantoea, Parachlamydia, Pasteurella, Pediococcus, Peptococcus, Peptostreptococcus, Phormidium, Photobacterium, Photorhabdus, Phyllobacterium, Phytoplasma, Planktothrix, Plesiomonas, Porphyromonas, Prevotella, Propionibacterium, Proteus, Providencia, Pseudoanabaena, Pseudomonas, Pseudonocardia, Pseudoramibacter, Psychrobacter, Rahnella, Ralstonia, Rhodococcus, Rickettsia, Rochalimaea, Roseomonas, Rothia, Ruminococcus, Salmonella, Schizothrix, Selenomonas, Serpulina, Serratia, Shewenella, Shigella, Simkania, Slackia, Sphaerotilus, Sphingobacterium, Sphingomonas, Spirillum, Spiroplasma, Spirulina, Staphylococcus, Stenotrophomonas, Stomatococcus, Streptobacillus, Streptococcus, Streptomyces, Succinivibrio, Sutterella, Suttonella, Tatumella, Tissierella, Trabulsiella, Treponema, Trichodesmium, Tropheryma, Tsakamurella, Turicella, Umezakia, Ureaplasma, Vagococcus, Veillonella, Vibrio, Weeksella, Wolinella, Xanthomonas, Xenorhabdus, Yersinia, and Yokenella.

Further preferred examples include bacteria selected from the following species: Acinetobacter baumannii, Actinobacillus actinomycetemcomitans, Actinobacillus pleuropneumoniae, Actinomyces bovis, Actinomyces Israelii, Bacillus anthracis, Bacillus ceretus, Bacillus coagulans, Bacillus liquefaciens, Bacillus popillae, Bacillus subtilis, Bacillus thuringiensis, Bacteroides distasonis, Bacteroides fragilis, Bacteroides thetaiotaomicron, Bacteroides vulgatus, Bartonella bacilliformis, Bartonella Quintana, Beneckea parahaemolytica, Bordetella bronchiseptica, Bordetella parapertussis, Bordetella pertussis, Borelia burgdorferi, Brevibacterium lactofermentum, Brucella abortus, Brucella canis, Brucella melitensis, Brucella suis, Burkholderia cepacia, Burkholderia mallei, Burkholderia pseudomallei, Campylobacter fetus, Campylobacter jejuni, Campylobacter pylori, Cardiobacterium hominis, Chlamydia pneumoniae, Chlamydia psittaci, Chlamydia trachomatis, Chlamydophila abortus, Chlamydophila caviae, Chlamydophila felis, Chlamydophila pneumonia, Chlamydophila psittaci, Chryseobacterium eningosepticum, Clostridium botulinum, Clostridium butyricum, Clostridium coccoides, Clostridium dijficile, Clostridium leptum, Clostridium tetani, Corynebacterium xerosis, Cowdria ruminantium, Coxiella burnetii, Edwardsiella tarda, Ehrlichia sennetsu, Eikenella corrodens, Elizabethkingia meningoseptica, Enterobacter aerogenes, Enterobacter cloacae, Enterococcus faecalis, Escherichia coli, Escherichia hirae, Flavobacterium meningosepticum, Fluoribacter bozemanae, Francisella tularensis, Francisella tularensis biovar Tularensis, Francisella tularensis subsp. Holarctica, Francisella tularensis subsp. nearctica, Francisella tularensis subsp. Tularensis, Francisella tularensis var. palaearctica, Fudobascterium nucleatum, Fusobacterium necrophorum, Haemophilus ducreyi, Haemophilus influenzae, Helicobacter pylori, Kingella kingae, Klebsiella mobilis, Klebsiella oxytoca, Klebsiella pneumoniae, Lactobacillus acidophilus, Lactobacillus casei, Lactobacillus hilgardii, Lactobacillus pentosus, Lactobacillus plantarum, Lactobacillus rhamnosus, Lactococcus lactis, Legionella bozemanae corrig., Legionella pneumophila, Leptospira alexanderi, Leptospira borgpetersenii, Leptospira fainei, Leptospira inadai, Leptospira interrogans, Leptospira kirschneri, Leptospira noguchii, Leptospira santarosai, Leptospira weilii, Leuconostoc lactis, Leuconostoc oenos, Listeria ivanovii, Listeria monocytogenes, Moraxella catarrhalis, Morganella morganii, Mycobacterium africanum, Mycobacterium avium, Mycobacterium avium subspecies paratuberculosis, Mycobacterium bovis, Mycobacterium bovis strain BCG, Mycobacterium intracellulare, Mycobacterium kansasii, Mycobacterium leprae, Mycobacterium marinum, Mycobacterium tuberculosis, Mycobacterium typhimurium, Mycobacterium ulcerans, Mycoplasma hominis, Mycoplasma mycoides, Mycoplasma pneumoniae, Neisseria gonorrhoeae, Neisseria meningitidis, Neorickettsia sennetsu, Nocardia asteroides, Orientia tsutsugamushi, Pasteurella haemolytica, Pasteurella multocida, Plesiomonas shigelloides, Propionibacterium acnes, Proteus mirabilis, Proteus morganii, Proteus penneri, Proteus rettgeri, Proteus vulgaris, Providencia alcalifaciens, Providencia rettgeri, Pseudomonas aeruginosa, Pseudomonas mallei, Pseudomonas pseudomallei, Pyrococcus abyssi, Rickettsia akari, Rickettsia canadensis, Rickettsia canadensis corrig, Rickettsia conorii, Rickettsia montanensis, Rickettsia montanensis corrig, Rickettsia prowazekii, Rickettsia rickettsii, Rickettsia sennetsu, Rickettsia tsutsugamushi, Rickettsia typhi, Rochalimaea quintana, Salmonella arizonae, Salmonella choleraesuis subsp. arizonae, Salmonella enterica subsp. Arizonae, Salmonella enteritidis, Salmonella paratyphi, Salmonella typhi, Salmonella typhimurium, Selenomonas nominantium, Selenomonas ruminatium, Serratia marcescens, Shigella boydii, Shigella dysenteriae, Shigella flexneri, Shigella sonnei, Spirillum minus, Staphylococcus aureus, Staphylococcus epidermidis, Staphylococcus equi, Staphylococcus lugdunensis, Stenotrophomonas maltophila, Streptobacillus moniliformis, Streptococcus agalactiae, Streptococcus bovis, Streptococcus ferus, Streptococcus pneumoniae, Streptococcus pyogenes, Streptococcus viridans, Streptomyces ghanaenis, Streptomyces hygroscopicus, Streptomyces phaechromogenes, Treponema carateum, Treponema denticola, Treponema pallidum, Treponema pertenue, Vibrio cholerae, Vibrio parahaemolyticus, Vibrio vulnificus, Xanthomonas maltophilia, Yersinia enterocolitica, Yersinia pestis, Yersinia pseudotuberculosis, and Zymomonas mobilis.

Further preferred examples include bacteria selected from the class of bacteria known as Fusospirochetes.

In the present invention, “fungi” are defined as any one of a large domain of single-celled eukaryotic microorganisms such as yeasts. As used herein, fungi include any that are known to those of ordinary skill in the art and any that may be discovered. Preferred examples of fungi are those known to be pathogenic to humans, animals or plants. Other preferred examples include those known to cause undesirable contamination and/or clogging of industrial flow systems. Still other preferred examples of fungi include those known to infect implanted medical devices (e.g., pumps, stents, artificial joints, screws, rods, and the like). Further preferred examples of fungi include those capable of forming biofilms and/or biostreamers. Further preferred examples include fungi selected from the genera: Candida, Saccharomyces, and Cryptococcus.

In the present invention, an “autoinducer” is defined as a molecule that activates or represses the expression of QS regulated genes. An “agonist” is defined as a naturally produced or synthetic autoinducer molecule that activates the expression of QS regulated genes. An “antagonist” is defined as a naturally produced or synthetic autoinducer molecule that represses the expression of QS regulated genes. Both agonists and antagonists are QS modulating molecules.

In the present invention, “biofilms” are defined as sessile microorganism community, such as a bacterial and/or fungal communities, that occupies a surface. These biofilms can cause chronic and medical device-associated infections, clogging, and/or device failure. Biofilms are surface-associated assemblies of microorganisms, such as bacteria and/or fungi which are bound together by extracellular polymeric substances (4, 5). Biofilms are attached to the surface all along the edges, including the bottom edge, of the surface. Although bacterial biofilms are desirable in waste-water treatment (6), biofilms primarily cause undesirable effects such as chronic infections or clogging of industrial flow systems (1-3). Cells in biofilms display many behavioral differences from planktonic cells, such as a 1,000-fold increase in tolerance to antibiotics (7, 8), an altered transcriptome (9-11), and spatially heterogeneous metabolic activity (12, 13). Some of these physiological peculiarities of biofilm-dwelling cells may be due to strong gradients of nutrients and metabolites, which also affect biofilm morphology and composition (14, 15).

In the present invention, “biofilm streamers” are defined as biofilms that have been partially detached from the surface upon which the biofilm is growing. Under conditions of flow in the presence of available biofilm promotion element(s) (e.g., curves, corners, bends, etc.), the flow partially detaches the extra cellular matrix off of the substrate along with cells that were in it already and is suspended in the liquid attached only at its edges. The detached biofilm forms filaments or streamers in the flowing liquid. The streamer is then able to capture other flowing debris and cells in order to continue growing. Thus, biofilms grow by cellular division, while biofilm streamers grow both by cell division as well as cellular capture of passing cells in the flow.

In the present invention, “biofilm growth” is defined as the expansion of the surface-attached biofilm over time, whether through cell division or through attachment of additional cells to the surface from the surrounding environment. As used herein, this growth includes expansion laterally over available surfaces as well as expansion through thickening of the biofilm layer by layers of additional cells.

In the present invention, “biofilm morphology” is defined as the physical composition or shape of the biofilm. As used in the invention, biofilm morphology may change over time. These changes may be in the composition of the extracellular matrix, in the composition of microorganisms, such as bacteria and/or fungi in the biofilm, or in the shape of the biofilm. Biofilm growth would be an example of a change in biofilm morphology. Another example of a change in biofilm morphology would be the flow induced formation of biofilm streamers. A third example would be the inclusion or expulsion of different microbial species within the biofilm.

In the present invention, “biofilm streamer growth” is defined as the expansion of the biofilm streamer over time. As used herein, this expansion may be in the length of the biofilm streamer filaments and/or in the thickness of the biofilm streamer. This growth may be through cell division and/or through capture of additional cells, extracellular matrix, and/or debris from the surrounding liquid.

In the present invention, “biofilm streamer morphology” is defined as the physical composition and/or shape of the biofilm streamer. As used in the invention, biofilm streamer morphology may change over time. These changes may be in the extracellular matrix, in the composition of the microorganisms (e.g., bacteria and/or fungi) in the biofilm streamer and/or in the shape of the biofilm streamer. Biofilm streamer growth, flow induced formation and/or inclusion/exclusion of different microbial species are all examples of a change in biofilm streamer morphology.

In the present invention, “QS modulator attached surface” is defined as any surface that possesses or is attached to a molecule that modulates QS, and in turn, alters any QS phenotype including, but not limited to, a biofilm, a biofilm streamer, and/or a virulence factor production via a linker. The surface may be any suitable solid surface or solid porous surface as is known to one of ordinary skill in the art. In one example, and in no way limiting, the surface is a glass coverslip. In a further example, the surface can be glass, stainless steel, plastic, polymers, sand, wire mesh, bone, teeth, skin, or blood vessels. In other examples, the surface will line a channel, e.g., the tubing of a fluid handling system.

As used herein, a “QS antagonist attached surface” is defined as any substrate that possesses or is attached to a molecule that antagonizes (e.g., inhibits or reduces) QS, and in turn, alters any QS phenotype including, but not limited to, a biofilm, a biofilm streamer, and/or a virulence factor production via a linker. Examples of QS antagonists are described in U.S. Pat. No. 8,247,443, U.S. Pat. No. 8,568,756, or PCT/U.S. Ser. No. 14/56497 which are specifically incorporated by reference in their entirety. See, for example, the structures described in FIGS. 2, 8 and 9 of U.S. Pat. No. 8,247,443, FIGS. 3A-P, 4A, 8A-8L and 10A-B of U.S. Pat. No. 8,568,756, and in Tables 1-4 and FIGS. 1, 6, 7, 12-15 of PCT/U.S. Ser. No. 14/56497, all of which are herein incorporated by reference in their entirety. Additionally, other preferred examples of QS antagonists include, but are not limited to small organic molecules, peptides and synthetic molecules.

Alternatively, a “QS agonist attached surface” is defined as any surface that possesses or is attached to a molecule that agonizes (e.g., promotes or increases) QS, and in turn, alters any QS phenotype including, but not limited to, a biofilm, a biofilm streamer, and/or a virulence factor production via a linker. Examples of QS agonists are described in U.S. Pat. No. 5,353,689 and PCT/US2014/051648 both of which are incorporated by reference in their entirety.

As used herein, “QS phenotype” or “morphology” or “trait” refers to any change in the bacterial colony/organism or in the constituents in the cells in the colony, including but not limited to, changes in appearance, e.g., an increase in streamer formation, a decrease in streamer formation, an increase in biofilm density, a decrease in biofilm density, etc. as well as other changes e.g., a change in gene expression, a change in mRNA production, a change in protein production, etc.

As used herein, “click chemistry” is a term to describe reactions that are high yielding, broadly applicable, create only byproducts that can be removed without chromatography, are stereospecific and generally simple to perform, and can be conducted in easily removable or benign solvents. In some embodiments, click chemistry allows generation of large libraries of compounds for screening in research. In one example, click chemistry enables covalent bond formation between molecule A with an azide group and with molecule B with an alkyne group. Click chemistry uses Cu catalysts to form triazoles by cycloaddition. A molecule with a PEG linker and an azide at one end may be reacted with another group (an anti-QS molecule) with an alkyne group attached at one end. Other methods are also possible, for example, the PEG linker can have an alkyne group attached, and the QS modulating molecules can possess azide groups at one end.

Thus, the inhibition of QS, biofilm, biofilm streamer, and/or a virulence factor production and/or morphology/phenotypic changes through the use of a QS antagonist may lead to either a decrease or an increase in overall virulence to the host depending on whether the microorganism relies on QS, biofilm, biofilm streamer, and/or a virulence factor production to promote infection. Similarly, the promotion of QS, biofilm, biofilm streamer, and/or a virulence factor production and/or morphology/phenotypic changes through the use of a QS agonist may lead to either a decrease or an increase in overall virulence to the host depending on whether the microorganism relies on QS, biofilm, biofilm streamer, and/or a virulence factor production to promote infection.

In the present invention, the surface can be made of any material. For example, glass, metals, including, but not limited to stainless metals, silicon, plastic, polymers, metals, and/or ceramic materials can be used.

Preferred examples of surfaces that can be used include, but are not limited to a surface comprising polymers, such as, for example, polyethylene, polypropylene, polystyrene, polyester, polyester PLA and other biosorbable plastics, polycarbonate, polyvinyl chloride, polyethersulfone, polyacrylate (e.g., Acrylic, PMMA), hydrogel (e.g., acrylate), polysulfone, polyetheretherketone, thermoplastic elastomers (e.g., TPE, TPU), thermoset elastomers, silicone, poly-p-xylylene (e.g., Parylene), fluoropolymers, a metal, including, but not limited to stainless steel, cobalt-base alloys, titanium, titanium-base alloys, and/or shape memory alloy, and/or a ceramic material including, but are not limited to glass ceramics, calcium phosphate ceramics, and/or carbon-based ceramics). Moreover, the surface can have any shape, such as, for example, small particles, including but not limited to nanoparticles, and/or flat and/or curved surfaces as described herein.

In the present invention, the surface may comprise a “biofilm promoting or biofilm streamer promotion element” as defined as any feature of the local environment in a flow system that, in the presence of pressure driven flow, serves as the site for biofilm formation, biofilm streamer formation or virulence factor production. For example, these biofilm streamer promotion elements may be roughened surfaces along the flow path, may be curves in the channel directing the flow, may be a turn in the channel directing the flow, may be a corner in the channel directing the flow, may be an edge or mound projecting into the lumen of the channel directing the flow, may be a constriction or expansion in the channel directing the flow, and/or may be provided by an object placed within the channel directing the flow.

In the present invention, the surface may comprise of a “channel” which is defined as a passage directing the flow of a fluid. As used in the invention, a channel may be an enclosed hollow tube. The cross section of the tube may be of any suitable geometry as is known by those of skill in the art. In one example the cross section is circular, oval, square, rectangular and/or irregularly shaped. The tube may have a constant cross-sectional area and/or it may be variable (e.g. it may constrict in certain areas and/or expand in others). The cross section of the channel may change shape along its length. In other examples, the channel may be a depression, gutter, groove and/or furrow. This depression may be shallow, deep, narrow and/or wide. In other examples, the channel may be provided by the gap between two parallel flat planar surfaces placed close together. In still other examples, the channel may be part of a larger device or machine or biological tissue or organ (e.g., lungs). The channel may be a flow conduit in an implantable medical device. The channel may also be a flow conduit in machinery used in industrial processes. The channel may be very small (i.e. just large enough for fluid and bacterial or fungal cells to flow through) or very large (i.e. the large culverts and pools used in a waste water treatment facility.)

In the present invention, “lumen” is defined as the area in a channel that is designed to direct the flow. It is defined as the interior of an enclosed hollow tube. It is the depressed area in a depression, gutter, groove or furrow. And, it is the gap between the adjacent parallel plates.

In the present invention, “circular” as applied to surface and/or a channel is defined as having a generally round cross sectional shape. As used in the present invention, circular does not require a perfectly circular cross section. In another example, circular means that the length of the diameter measured anywhere along the cross section of the channel is identical to that measured at any other point (i.e. it is perfectly circular).

In the present invention, “turn” is defined as a portion of a surface with a defined, discrete change in direction of the flow. This turn may be of any degree. In one example, the turn is a change in direction from about 210° to about 360°, more preferably from about 220° to about 350°, more preferably from about 230° to about 340°, more preferably from about 240° to about 330°. As used herein, a turn may be rounded or may be sharp. When a turn is sharp, it may result in a corner.

In the present invention, “corner” is defined as the point or area where two lines, edges, or sides of something meet. A corner may be an edge formed by a turn in the channel A corner may also be a raised point, for example, in the lumen of the channel For example, a pyramidal obstruction, placed in the lumen of the channel such that the base is against the surface of the channel and the tip is directed towards the center of the channel, would form a corner.

In the present invention, “edge” is defined as a line or line segment that is the intersection of two plane faces. An edge may be formed on a surface, such as in a channel along the inside surface of a turn. An edge may also be formed by a raised surface, for example, in the lumen of the channel For example, a raised wedge, placed on a surface, such as in the lumen of the channel, such that the base is against the surface of the channel and the raised edge of the wedge is directed towards the center of the channel, would form an edge. The edge formed by this wedge obstruction could be placed perpendicular to the fluid flow or parallel to the flow.

In the present invention, “mound” is defined as a raised area on a surface, such as for example, within the lumen of the channel, without any appreciable corners or edges. A used herein, a mound would be a generally curved obstruction on a surface. In one example, the mound is a raised circular bump. In another example, the mound may be formed by placing a half-cylinder (formed by cutting perpendicular to its circular faces) onto a surface. For example, a mound in a channel would be positioned such that a flat surface is placed against the surface of the channel with the circular surface faced towards the center of the channel A further example includes a cylinder placed such that the semicircular top and bottom are perpendicular to the flow or parallel to the flow.

In the present invention, “roughened surface” is defined as an irregular surface. It may be a surface that microscopically reduces to surfaces with many corners. In another example, it may a surface with distinct geometric and irregular deformities on a macroscopic level.

In the present invention, an “object” comprises a surface. In some examples, an object may include, for example, sand, gravel, granules and the like. In other examples, an object may include portions of medical devices or industrial fluid handling machinery. For example, an object may include filter support grids, filter mesh, stents, tubing or channel components for fluid handling, valves, pumps, and the like. These objects may be of any scale from miniature components of implantable medical devices to large scale fluid handling components of industrial cooling units or food processing machinery.

In the present invention, “NAFION® granules” are defined as amorphous particles of fluoropolymer first discovered in the late 1960s by DuPont. In one example, the granules are of the size and shape of sand. In another embodiment, the granules may be larger, including up to the size of grains of rice, stones or boulders. As used herein, one of ordinary skill in the art will recognize that additional fluoropolymers may be employed in the invention. In further examples, Teflon AF, Teflon FEP and CYTOP may each be used as part of the surface of the invention (47).

In the present invention, “welded polypropylene feed spacer mesh” is defined as mesh similar to that used in industrial reverse osmosis filters (48). As used herein, this mesh may be any porous mesh used in industrial, medical, or other fluid handling applications.

In the present invention, “stent” is defined as a mesh tube inserted into a natural passage/conduit in the body to prevent localized flow constriction.

In the present invention, “bare-metal stent” is defined as type of vascular stent without a coating (as used in drug-eluting stents, for example). Stents are made out of different types of fabrics, polymers, and other materials, such as for example, bare stainless steel or may be made of alloys (e.g., cobalt chromium).

In the present invention, “pipe” is defined as a generally rigid tube used to convey fluid or compressed gases. A pipe may have an inner diameter as small as 2 mm or as large as several feet. A pipe is made of glass, any number of metals, any number of plastics or other polymeric materials, or concrete. A pipe as used herein may be any that is known to one of ordinary skill in the art.

In the present invention, “cooling tower” is defined as a heat rejection device, which extracts waste heat to the atmosphere through the cooling of a water stream to a lower temperature. The type of heat rejection in a cooling tower is termed “evaporative” in that it allows a small portion of the water being cooled to evaporate into a moving air stream to provide significant cooling to the rest of that water stream.

In the present invention, “fluid” is defined as a liquid or a gas. In one example, the fluid is water, with or without the addition of other components. These additional components may include, but are not limited to nutrients and salts needed to support bacterial growth, bacteria, chemical or biochemical probes to assist with visualization of cells or extracellular components, test compounds, and compounds for selective growth of specific bacterial strains. In other embodiments, a fluid is a biological fluid such as, for example, blood.

In the present invention, “flow” or “fluid flow” is defined as movement of the fluid along a surface in a continuous stream.

In the present invention, “flow rate” is defined as the volume of a fluid moving along a surface per unit time.

In the present invention, “Reynolds number” is defined as a dimensionless quantity used to help predict similar flow patterns in different fluid flow situations. It is defined as the ratio of inertial forces to viscous forces and thus quantifies the relative importance of these two types of forces for given flow conditions. Reynolds numbers may be used to characterize different flow regimes within a similar fluid, such as laminar or turbulent flow. When a fluid is flowing through a surface, such as a closed channel such as a pipe or between two flat plates, either of two types of flow may occur depending on the velocity of the fluid: laminar flow or turbulent flow. Laminar flow tends to occur at lower velocities, below a threshold at which it becomes turbulent. A Reynolds number of less than 2320 is characteristic of laminar flow in a circular tube. A Reynolds number greater than 2320 is characteristic of turbulent flow in a circular tube.

In the present invention, “laminar flow” in a long straight surface is defined as a flow regime that occurs when a fluid flows in parallel layers, with no disruption between the layers. At low velocities, the fluid tends to flow without lateral mixing, and adjacent layers slide past one another like playing cards. For flow in a long straight surface, such as a long straight channel, there are no cross-currents perpendicular to the direction of flow, nor eddies or swirls of fluids. In laminar flow, the motion of the particles of the fluid is very orderly with all particles moving in straight lines parallel to the pipe walls. For flows in more complicated geometries, such as channels with bends and corners, the laminar flow is the time-independent motion for a steady pressure drop; the flow may be three-dimensional, i.e. the velocity may have all three components non-zero, but the flow remains steady (time independent) so long as the pressure drop is constant.

In the present invention, “turbulent flow” is defined as a flow regime characterized by chaotic property changes. This includes low momentum diffusion, high momentum convection, and rapid variation of pressure and velocity in space and time. In turbulent flow, unsteady vortices appear on many scales and interact with each other. Drag due to boundary layer friction increases. The structure and location of boundary layer separation often changes, sometimes resulting in a reduction of overall drag.

In the present invention, “shear stress” is defined as the force/area acting tangent to a surface. In an ordinary fluid such as water the shear stress is proportional to the fluid viscosity and proportional to the velocity gradient (as defined in standard textbooks).

In the present invention, “controlled pressure” is defined as pressure applied to a fluid moving through a channel such that the pressure drop along the channel is held constant. Thus, as resistance to flow in the pipe is increased, rather than continuing to apply increasing pressure to keep the flow rate constant, the flow rate is reduced such that the pressure remains constant. As used herein, a constant pressure includes pressure that varies. For example, the pressure may “pulse” at a given frequency, for example, but the average pressure will remain constant.

In the present invention, “time until clogging (T)” is defined as the time at which the fitted flow rate drops to half its initial value.

In the present invention, “duration of the clogging transition (τ)” is defined as the time period in which the fitted flow rate decreases from 76% to 27% of its initial value.

In the present invention, “test compound” is defined as any compound added to the test system for evaluation of its effect on QS, biofilm formation, biofilm streamer formation, and/or a virulence factor production. The effect of the test compound may be to inhibit (an antagonist) or to enhance (an agonist) QS, biofilm, biofilm streamer, and/or a virulence factor production and/or morphology changes. The inhibition of QS, biofilm, biofilm streamer, and/or a virulence factor production and/or morphology changes through the use of a QS antagonist may lead to either a decrease or an increase in overall virulence to the host depending on whether the microorganism relies on QS, biofilm, biofilm streamer, and/or a virulence factor production to promote infection. Similarly, the promotion of QS, biofilm, biofilm streamer, and/or a virulence factor production and/or morphology changes through the use of a QS agonist may lead to either a decrease or an increase in overall virulence to the host depending on whether the microorganism relies on QS, biofilm, biofilm streamer, and/or a virulence factor production to promote infection.

These compounds may be pharmaceutical compound, small molecules, or biological compounds. Some examples include peptides, proteins, peptidomimetics, antibodies, non-antibody specific binding molecules, such as adnectins, affibodies, avimers, anticalins, tetranectins, DARPins, mTCRs, engineered Kunitz-type inhibitors, nucleic acid aptamers and spiegelmers, peptide aptamers and cyclic and bicyclic peptides and small synthetic or natural organic molecules (Ruigrok et al. Biochem J. (2011) 436, 1-13; Gebauer et al., Curr Opin Chem Biol. (2009) (3):245-55.)

In the present invention, “antibody” refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments as well as variants (including derivatives) of antibodies and antibody fragments. Examples of molecules which are described by the term “antibody” in this application include, but are not limited to: single chain Fvs, and fragments comprising or alternatively consisting of, either a VL or a VH domain. The term “single chain Fv” or “scFv” as used herein refers to a polypeptide comprising a VL domain of an antibody linked to a VH domain of an antibody. Antibodies of the invention include, but are not limited to, monoclonal, multispecific, human or chimeric antibodies or antibodies made in animals, single chain antibodies, Fab fragments, F9ab′) fragments, antiidiotypic (anti-Id) antibodies (including, e.g., anti-Idantibodies to antibodies of the invention), and epitope-binding fragments of any of the above. The immunoglobulin molecules of the invention can be of any type (e.g., IgG, IgE, IgM, IgD, IgA, and IgY), class (e.g., IgG1, IgG2, IgG3, IgG4, IgA1 and IgA2) or subclass of immunoglobulin molecule.

As used herein, “average surface coverage density” refers to the number of QS modulating molecules attached to the surface per unit area. See also, Example 14.

As used herein, “Gram-positive” refers to a type of bacteria surrounded by a thick layer of peptidologlycan. Gram-positive bacteria include staphylococci (“staph”), streptococci (“strep”), pneumococci, and the bacterium responsible for diphtheria (Cornynebacterium diphtheriae) as well as anthrax (Bacillus anthracia). Gram-positive bacteria react with the Gram stain to turn dark blue or violet.

As used herein, “Gram-negative” refers to bacteria that have a cytoplasmic membrane, a thin peptidoglycan layer, and an outer membrane containing lipopolysaccharide. Gram-negative bacteria do not react with the Gram stain to turn dark blue or violet, instead these bacteria appear red or pink due to counterstain (usually safranin).

As used herein, “peptidoglycan layer” refers to an elastic polymeric mesh-like network found outside the bacterial cell membrane.

As used herein, “linker length” means the longitudinal length of the linker, usually in nm.

As used herein, “linker diameter” means the diameter or breadth of the linker.

As used herein, “permeability agent” means any agent capable of forming holes in the outer membrane layer of gram-negative bacteria. Examples include holins, endolysins, or bacteriocins⁵⁰.

As used herein, the terms “a” or “an” are used, as is common in patent documents, to include one or more than one, independent of any other instances or usages of “at least one” or “one or more.”

As used herein, the term “or” is used to refer to a nonexclusive or, such that “A or B” includes “A but not B,” “B but not A,” and “A and B,” unless otherwise indicated.

As used herein, the term “about” is used to refer to an amount that is approximately, nearly, almost, or in the vicinity of being equal to or is equal to a stated amount, e.g., the state amount plus/minus about 5%, about 4%, about 3%, about 2% or about 1%.

### B. Agr QS

S. aureus Agr QS is driven by an autoinducer peptide (called AIP) harboring a thiolactone ring and an exocyclic tail at the N-terminus. AIP is processed from the precursor peptide AgrD by AgrB and other proteases, and the AIP is secreted^{26,27}. Extracellular AIP is detected by a cognate transmembrane-bound receptor histidine kinase, AgrC, that upon AIP binding, autophosphorylates and subsequently funnels a phosphoryl group to the partner response regulator, AgrA^{28,29} (FIG. 1a). Phospho-AgrA activates the agrP3 promoter driving transcription of RNAIII that has multiple roles³⁰. RNAIII functions as an mRNA that encodes δ-toxin (a membrane disrupting exo-protein that lyses eukaryotic host cells), and RNAIII also participates in regulation of other genes required for exo-toxin secretion and biofilm disassembly³¹. Detection of AIP launches the autoinduction positive feedback loop that increases AIP production, resulting in amplification of the QS response⁸.

There are four S. aureus Agr allelic variants (I to IV) that make four AIPs differing only in a few amino acid residues. AIPs activate QS in the S. aureus cells that produce them, and they generally inhibit QS in heterologous S. aureus cells possessing different AIP variants⁸. In the S. aureus agr-I strain referenced herein, AIP-I is the native autoinducer.

TrAIP-II, a truncated AIP-II with the exocyclic tail replaced by an acetyl group, is a universal inhibitor for all four S. aureus Agr QS systems³². TrAIP-II competes with the cognate AIPs for binding to the receptor³². Unless otherwise indicated, AIP-I acts as an autoinducer agonist and TrAIP-II acts as a competitive antagonist to S. aureus agr-I (FIG. 1a). S. aureus agr-I was selected for study because it possesses the most prevalent Agr type found world-wide in nosocomial infections.

### C. Uses of QS Modulator Attached Surfaces

The compositions described herein can be used to treat surfaces to modulate QS, biofilm formation, biofilm streamer formation, and/or virulence factor production or any other QS-controlled trait of interest. For example, the invention can reduce and/or prevent QS, biofilm formation, biofilm streamer formation, and/or virulence factor production on structures most susceptible to colonization and/or clogging by microorganisms by attaching a QS modulating molecule to the surface. In one embodiment, the QS modulator attached surface may be used to treat component parts or particular materials in microfluidic or other benchtop-sized assay systems. In another embodiment, the QS modulator attached surface may be used to treat industrial fluid handling systems or other areas where fluid is directed along channels. In a further embodiment, a QS modulator can be coated on any surface made of any material in medical devices or tools used in medicine. In a further embodiment, various types of QS modulator molecules and/or antibiotics and/or enzymes and/or antibodies can be simultaneously coated onto the same surface to target various microorganisms at the same time. In a further embodiment, the surface coated with a QS modulating molecule is resistant to multiple infections or colonization events. In a further embodiment, methods of screening for agonists or antagonists of QS, biofilm formation, biofilm streamer formation, and/or virulence factor production can be performed using a surface with a test compound linked to the surface. These screens may additionally be run in the presence of various antibiotics to detect effectors that enhance antibiotic inhibition. In a further embodiment, methods for detecting microorganisms can be performed using a surface coated with a QS modulator molecule.

For example, the QS modulator attached surface, and preferably a QS antagonist attached surface, has applications for any natural or artificial surface where the presence of a microorganism, such as, for example, S. aureus could be detrimental. These compositions have immediate applications for medical and health-care devices in which a microorganism, such as for example, S. aureus, colonizes. The QS modulating molecule attached surface as described herein are expected to inhibit biofilm formation, biofilm streamer formation and/or toxin synthesis of a microorganism by interfering with the QS regulatory network, thereby reducing the severity of infection and/or colonization caused in these devices. Furthermore, the QS antagonist attached surface are not prone to bacterial antibiotic-resistance, leading to improved treatment for the bacterial infections. The QS antagonist attached surface can be applied to other devices in which bacterial or other microorganism contamination is a concern. Examples include water supply lines, filters, stents, and intubation tubes, etc. In addition, the QS antagonist attached surface can affect a broad range of other Gram-positive and/or Gram-negative pathogens that use QS pathways to control virulence, including but not limited to, Staphylococcus epidermidis, Streptococcus pneumonia, Streptococcus mutans, and Streptococcus sanguinis.

More specifically, QS modulating molecule attached surface can be used in industrial settings, either in the presence or absence of antibiotics, to inhibit or prevent QS, biofilm formation, biofilm streamer formation, and/or virulence factor production and/or to remove antibiotic resistant bacteria, such as in a hospital or other public setting. For example, the QS antagonist attached surface can be used to remove biofilms that have grown on hospital surfaces, in moist and warm environments, such as showers, water and sewage pipes, cooling or heating water systems, (e.g., cooling towers), marine engineering systems, such as, for example, pipelines of the offshore oil and gas industry. The QS antagonist attached surface can also be used, for example, to remove and/or prevent bacterial adhesion to boat hulls, since once a biofilm of bacteria forms, it is easier for other marine organisms such as barnacles to attach. The QS antagonist attached surface can be used to reduce, for example, the time a boat is in dry dock for refitting and repainting, thereby increasing productivity of shipping assets, and useful life of the ships. The QS antagonist attached surface can also be used to remove biofilm production intentionally used to eliminate petroleum oil from contaminated oceans or marine systems, once the contamination is removed.

Additionally, the QS antagonist attached surface can be used to wash, rinse or swab floors and counters, such as in food preparation areas or medical facilities, as well as medical devices, including but not limited to, stents, catheters, intubation tubes, or ventilator equipment. Still further, the QS antagonist attached surface can be used as a handwash to help eliminate spread of virulent bacteria by health workers, patients and others.

The QS modulator attached surface can also be used to coat an implantable medical device, part of machinery used in industrial processes, a culvert, a pool used in a waste water treatment facility, waste water treatment facility, a pipe, a cooling tower, a medical device, industrial fluid handling machinery, a wound, within the body, a medical process, agricultural processes, and/or machinery

Particular species of bacteria may be especially problematic. For example, Pseudomonas aeruginosa is a pathogen that can survive in a wide range of environments. The bacterium is a public health threat because it causes a variety of secondary infections in humans, where those with burn wounds, cystic fibrosis, and implanted medical devices and cancer patients receiving chemotherapy are particularly at risk. With an outer membrane of low permeability, a multitude of efflux pumps, and various degradative enzymes to disable antibiotics, P. aeruginosa is difficult to treat. As with other common pathogenic bacteria, antibiotic-resistant strains are an increasing problem.

Thus, the QS modulator molecule attached surface invention can be used to treat bacterial infections (e.g., sepsis or infections from cystic fibrosis) in a patient, particularly those bacterial inventions cause by antibiotic resistant strains. In preferred embodiments, the bacterial infections are caused by P. aeruginosa and/or S. aureus.

The surface attached QS modulating molecule can be used to positively and negatively manipulate QS in bacteria such as S. aureus. For example, agonists or antagonists of QS can be attached to a surface via a linker, such as polyethylene glycol (PEG) polymers, which are known to be flexible, hydrophilic, and non-bulky. These surface-attached molecules can successfully be used to control bacterial QS, which has potential implications for the development of anti-biofouling or anti-colonization materials in industry and medicine, respectively. In S. aureus, Agr QS activation leads to the production of a battery of virulence factors that are reponsible for invasion and dissemination in host tissues¹⁰. Agr QS in S. aureus also activates the biofilm disassembly process⁴². Thus, precisely manipulating Agr QS using synthetic strategies can terminate virulence while not enabling biofilm formation. Due to resistance of S. aureus, there is an urgent medical need for the control of S. aureus.

Antagonist-coated surfaces can be used in scenarios such as acute infections, e.g. staphylococcal scalded skin syndrome and toxic shock syndrome where it is essential to halt production of exo-toxins. Reciprocally, autoinducer-coated surfaces can be used to treat chronic infections, e.g. pneumonia or medical device-related infections, where S. aureus biofilms are the major issue. Indeed, S. aureus cells residing in surface-bound biofilms are more resistant to antibiotics and host immune defenses than are their planktonic counterparts⁴³. AIP-I-coated surfaces, by triggering biofilm dispersal and transitioning the S. aureus cells to the planktonic lifestyle, can render surfaces resistant to biofilm colonization and, furthermore, render the dispersing cells more susceptible to antibiotics and to host immune defenses.

The techniques provided herein for coating surfaces with pro- or anti-QS molecules and using them to influence bacterial behaviors are not limited to S. aureus. These strategies are generally applicable to any bacterial strain. For example, the Gram-positive bacterium Enterococcus faecalis causes life-threatening urinary tract infections, bacteremia, endocarditis, and meningitis in humans⁴⁴. Pathogenicity of E. faecalis relies on the Fsr QS system, which is homologus to the S. aureus Agr QS system⁴⁵. However, importantly, in the case of E. faecalis, activation of Fsr QS promotes both biofilm formation and virulence factor production^{45,46}. Thus, surfaces harboring Fsr QS antagonists (i.e., ZBz1-YAA5911⁴⁷) can have a dual benefit in preventing biofilms and reducing exo-toxin production. Such dual benefits can also be imagined for other bacteria such as Listeria monocytogenes and Streptococcus pyogenes, as both pathogens possess Agr-type QS systems that activate biofilm formation and virulence factor expression at high cell density⁴⁸. Moreover, the beneficial bacterium Lactobacillus plantarum, which is important in the dairy and fermented food industries, also has an Agr-type QS system called Lam that could be manipulated using surfaces in applications in food production⁴⁹. Thus, any bacterial system capable of QS can be modulated based on the techniques provided herein.

Clearly, the techniques provided herein have the potential to be expanded to other systems with known ligands and with accessible cognate receptors. This method is especically useful for Gram-positive bacteria because they do not possess an outer membrane. Strategies for Gram-negative bacteria can be explored by exploiting surfaces coated with QS-manipulation compounds together with molecules that form pores in the outer membrane such as holins, endolysins, or bacteriocins⁵⁰. Likewise, surface-attached QS molecules could be examined with other orthogonal approaches that exploit surface release of active compounds²². Present invention embodiments are not intended to be limited to the examples provided herein, and may be applicable in any bacterial system (or non-bacterial system) which utilizes QS.

Administration of the QS modulating attached surface to a subject is specifically contemplated. For example, a QS antagonist attached to a small particle/bead, such as, for example, a nanoparticle, can be administered to a patient to reduce or eliminate an infection.

Blocking virulence is one of the strategies contemplated to combat these bacteria. This approach provides less selective pressure for the spread of resistant mutants and leads to drug therapies that are effective over a greater time span compared to traditional antibiotics. Rather than preventing growth or killing the bacteria, an antivirulence approach prevents the expression of virulence traits. The bacteria that have been treated and are thus benign should then be more easily cleared by the host immune system.

The QS modulating molecule attached surface is resistant to multiple infections or colonization events by microorganisms. The surface can be simultaneously attached to various QS modulating molecules and/or antibiotics and/or enzymes.

Examples of QS modulating molecules are shown in Tables 1A-1B. Each of the references in Table 1A are included herein in their entirety.

TABLE 1A

| Patent No. | Reference | Compounds |
|---|---|---|
| U.S. application Ser. No. 7,419,954 | Table 1 | — |
| | General formula from col. 7 line | — |
| | 53 to col. 8, line 11 | |
| | FIGS. 1A and 6 | — |
| U.S. application Ser. No. 6,953,833 | General formula from col. 4 lines | — |
| | 43 to 67 | |
| | FIG. 1A | — |
| PCT/US2014/056497 | Table 1 | Entry 1-12 |
| | Table 2 | Entry 1-23 |
| | Table 3 | Entry 1-23 |
| | Table 4 | Entry 1-20 |
| | General formulas shown in | — |
| | paragraphs [0011], [0012], [0013] | |
| | and [0015] | |
| U.S. Pat. No. 8,568,756 | FIG. 3 | Antagonist 6807-0002 |
| (same antagonists also in | | Antagonist 8008-8157 |
| U.S. Pat. No. 8,772,331 and | | Antagonist Cl04-0038 |
| U.S. Pat. No. 8,247,443) | | Antagonist Cl05-2488 |
| | | Antagonist 3448-8396 |
| | | Antagonist 3578-0898 |
| | | Antagonist 3643-3503 |
| | | Antagonist 4052-1355 |
| | | Antagonist 4248-0174 |
| | | Antagonist 4401-0054 |
| | | Antagonist 4606-4237 |
| | | Antagonist C137-0541 |
| | | Antagonist C450-0730 |
| | | Antagonist C540-0010 |
| | | Antagonist C646-0078 |
| U.S. Pat. No. 8,247,443 | General formula shown in col. 7, | — |
| | lines 1-33 | |
| U.S. Pat. No. 8,535,689 | FIGS. 13a-13e | Compounds 1-33, and CAI-1 |
| WO 2014/092751 | FIG. 2A | Compounds 1-11 |
| | FIG. 3 | Compounds 11-18 |

Unless otherwise indicated, the compounds of PCT/US2014/056497 and U.S. Pat. No. 8,568,756 function as antagonists of QS to inhibit the QS pathway.

It is also expressly understood that the compounds referred to (and incorporated by reference) in PCT/US2014/056497 are limited to those that exhibit anti-pathogenic and anti-biofilm activity through inhibition of QS.

Unless otherwise indicated, the compounds of U.S. Pat. No. 8,535,689 and WO 2014/092751 function as agonists. Some QS systems, such as those found in cholera, work “in reverse” from other QS systems. For example, agonists of cholera QS receptors repress biofilm formation and pathogenicity, effectively functioning as inhibitors of bacterial infections.

Other molecules, including flavonoid compounds, function as antagonists of QS, and are shown in Table 1B.

TABLE 1B

| Com- | | |
|---|---|---|
| pound | Chemical | |
| No. | Name | Structure |
| #3 | chrysin | 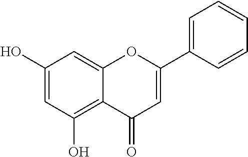 [SMILES: O=c1cc(-c2ccccc2)oc2cc(O)cc(O)c12] |
| #43 | apigenin | 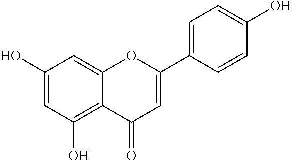 [SMILES: O=c1cc(-c2ccc(O)cc2)oc2cc(O)cc(O)c12] |
| #48 | quercetin | 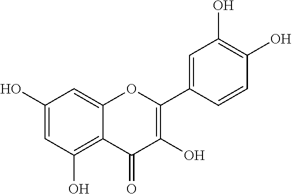 [SMILES: O=c1c(O)c(-c2ccc(O)c(O)c2)oc2cc(O)cc(O)c12] |
| #46 | baicalein | 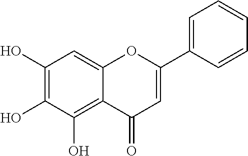 [SMILES: O=c1cc(-c2ccccc2)oc2cc(O)c(O)c(O)c12] |
| #54 | 7,8- dihydroxy- flavone | 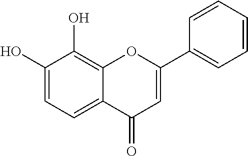 [SMILES: O=c1cc(-c2ccccc2)oc2c(O)c(O)ccc12] |
| #53 | 6- dihydroxy- flavone | 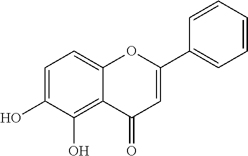 [SMILES: O=c1cc(-c2ccccc2)oc2ccc(O)c(O)c12] |
| #4 | narigenin | 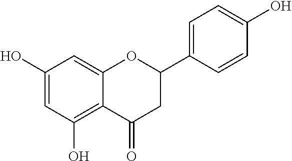 [SMILES: O=C1CC(c2ccc(O)cc2)Oc2cc(O)cc(O)c21] |
| #1 | phloretin | 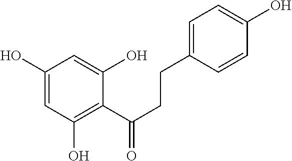 [SMILES: O=C(CCc1ccc(O)cc1)c1c(O)cc(O)cc1O] |
| #18 | 3,5,7- trihydroxy- flavone | 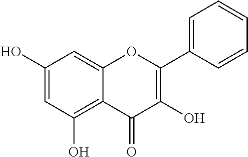 [SMILES: O=c1c(O)c(-c2ccccc2)oc2cc(O)cc(O)c12] |
| #19 | pinocembrin | 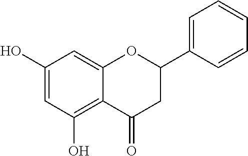 [SMILES: O=C1CC(c2ccccc2)Oc2cc(O)cc(O)c21] |

It is expressly understood that present invention embodiments include both agonists and antagonists. In some systems, compounds act as antagonists with respect to the QS system to repress biofilm formation and pathogenicity, while in other systems, compounds act as agonists with respect to the QS system to repress biofilm formation and pathogenicity.

### EXAMPLES

### Example 1

### Conjugation of Linker to Substrate

Any substrate can be used to conjugate a linker. For example, substrates can be any material, for example, but not limited to, polymers, metals, and/or ceramics. In this example, glass and gold substrates are used. Other examples of substrates are shown below in Table 2.

In some embodiments, linkers are chosen to be biocompatible and flexible. Linkers can be a variety of lengths and chemistries and they can contain different chemical moieties. The linker used in this study was Polyethylene Glycol (PEGs). Other examples of substrates are shown below in Table 3, but are in no way limiting.

TABLE 2

| Substrates | Polymers | Metals | Ceramic Materials |
|---|---|---|---|
| Examples | Nylon, PEEK (Polyetheretherketone), Polyethylene, | Stainless steel, | Glass Ceramics, |
| | Polypropylene, Polystyrene, Polyester, Polyester | Cobalt-Based | Calcium Phosphate |
| | (PLA and Other Biosorbable Plastics), Polycarbonate, | Alloys, | Ceramics, Carbon- |
| | Polyethane, Polyurethane, Polyvinyl Chloride, | Titanium and | Based Ceramics |
| | Polyethersulfone, Polyacrylate(Acrylic, PMMA), | Titanium- | |
| | Hydrogel (Acrylate), Polysulfone, Silicone | Based Alloys, | |
| | Thermoplastic Elastomers (TPE, TPU), Thermoset | Shape Memory | |
| | Elastomers-Silicone, Tygon, Poly-p-xylylene | Alloys | |
| | (Parylene), Fluoropolymers | | |

TABLE 3
Linkers

| | |
|---|---|
| Examples | Polyethylene Glycol (PEGs), Polyphosphazenes, Polylactide, |
| | Polyglycolide, Polycaprolactone, or any other combinations |
| | with these. |

Any specific chemistry can be used to make a chemical bond between a surface and a linker. Non-limiting examples include silanization, gold-sulfide bond formation, thiol-ene reactions, and surface-initiated polymerization.

In a preferred embodiment, the linker was covalently bound to the surface by using a free hydroxyl moiety on the glass substrate (e.g., see FIG. 14). For example, by using corona treatment (or air plasma) for 1 min at room temperature, the corona discharge plasma changed the properties of the glass surface to generate the reactive hydroxyl group. Alternatively, a mixture of sterile water, hydrogen chloride, and hydrogen peroxide (5:1:1 in v/v) was used to treat the glass surface for 10 min at room temperature. After the reaction, blowing nitrogen air and injection of water was used to remove unreacted chemicals. Alternatively, the glass slides were boiled for 2 hours in 3% sodium peroxodisulfate at 50° C. Subsequently, the glass slides were washed thoroughly, twice, with millipore water. The washed slides were submerged in piranha etch (3:1 H₂SO_{4:}H₂O₂) solution overnight. Subsequently, the slides were washed thoroughly, twice, with 150 mL millipore water and rinsed with acetone three times to remove any trace of water. The hydroxyl-containing glass substrate was conjugated to PEG linkers by one-step silanization (e.g., see FIG. 14). Specifically, commercially available PEG linkers with a trimethoxysilane moiety were used. This linker leaves a methoxy group, a good leaving group for the silanization reaction that forms the covalent bond between the linker and the hydroxyl glass substrate. Specifically, PEG linkers with trimethoxysilane were solubilized in acetone to obtain a concentration of 200 mg/ml. The linker solution was used to treat the hydroxyl glass substrate for 30 min-60 min at room temperature. Blowing nitrogen air and injection of water was used to remove unreacted linkers. The anti-QS molecule was then conjugated to the linker, e.g., as described in Example 2, and the resultant surface-attached molecule was capable of binding to the QS receptor of the S. aureus bacterial cell.

In the example of thiol-ene reactions (see, e.g., FIG. 15), a glass substrate having thiol moieties was rinsed with acetone twice, and subsequently rinsed with millipore water and dried. Subsequently, a solution that contained 200 mg of commercially available PEG linkers functionalized with a maleimide moiety in lml of 100 mM pH 7.2 Tris was added to the surface of the glass slide and the reaction was carried out overnight at 4° C. After the reaction was complete, the slides were washed with millipore water. After the PEG-based linker was attached to the substrate, QS agonist AIP-I was then attached to the linker, as described in Example 2, using e.g., click chemistry.

In the example of the gold-sulfide bond formation reaction, commercially available gold-coated glass slides were submerged in acetone for 5 min and then dried. (See, e.g., FIGS. 16A-16B). Alternatively, a gold covered slide may be generated by placing a coat of gold on a glass slide. This process was repeated three times. The slides were washed with isopropanol to remove any trace of acetone, and then dried. Subsequently, a solution that contained 200 mg of commercially available PEG linkers with a thiol moiety in 1 ml of water was added to the surface of the glass slides. A gold-sulfide bond was formed, rendering the PEG linker attached to the gold-plated surface via the gold-sulfide bond. Water was used to wash away any residue of unreacted linkers. An alkynated QS modulated molecule was then attached to the free end of the linker, as described in Example 2.

In another example of thiol-ene reactions (See, e.g., FIGS. 17A-17B), glass slides were submerged in piranha etch (H₂SO_{4:}H₂O₂) solution to generate a hydroxylated surface. The hydroxyl-containing glass substrate was submerged in 20% (3-Mercaptopropyl) trimethoxysilane in acetone for 60 min to generate —SH— moieties on the surface. The glass slide was rinsed with acetone twice, and was subsequently rinsed with millipore water and dried. Subsequently, a solution that contained 200 mg of commercially available PEG linkers functionalized with a maleimide moiety in 1 ml of 100 mM pH 7.2 Tris was added to the surface of the glass slide and the reaction was carried out overnight at 4° C. After the reaction was complete, the slides were washed with millipore water. The PEG-based linker was attached to the substrate. An alkynated QS modulated molecule was then attached to the free end of the linker, as described in Example 2.

In the example of the surface-initiated polymerization, RAFT (reversible-addition fragmentation chain transfer) polymerization was used, but in no way is limiting. The polymerization involved radical formation of a monomer in the presence of a RAFT agent such as a trithiocarbonate compound. In one preferred method, N-(2-hydroxylpropyl)methacrylamide (HPMA) was used and synthesized in the following way: 36 g (0.34 mol) of anhydrous sodium carbonate (Na₂CO₃) was suspended in 85 ml (freshly distilled) methylene chloride (CH₂Cl₂). The solution was cooled to −10° C. (using dry ice), and 25 ml (0.30 mol) of 1-aminopropan-2-ol was added. The suspension was maintained at −10˜° C., and (freshly distilled) 31.9 ml (0.29 mol) methacryloyl chloride was added drop-wise under cooling and vigorous stirring within 60 min. The reaction mixture was stirred for another 20˜30 min at 5˜15° C. 10 g of anhydrous sodium sulfate (Na₂SO₄) was added, the solid was filtered twice and the dry filtrate was concentrated to half volume under reduced pressure until formation of a crystallization seed. HPMA was obtained by crystallization from methylene chloride at −20° C. overnight, and purified by recrystallization from acetone.

In a preferred method, N-(Azido(PEG)) methacrylamide (AzPMA) was used and synthesized in the following way: 1.1 g (11 mmol) of Triethylamine (N(CH₂CH₃)₃) was dried over MgSO₄ and together with 1 g (1 ml, 4.53 mmol) of 11-Azido-3,6,9-trioxaundecan-1-amine, 3.4 mg of hydroquinone and dry 7.5 ml of methylene chloride (CH2Cl2) cooled to 0° C. in an ice-water bath. 1 mL (10 mmol) of methacryloyl chloride was added dropwise to the reaction mixture over 20 min followed by stirring at 0° C. for 1 hour and at room temperature for 14 hours. 3.6 ml of methylene chloride (CH₂Cl₂) was added and the mixture was washed twice with aqueous HCl (2 M), aqueous NaOH (2 M), and water (15 mL). The organic phase was dried over anhydrous MgSO_{4,} and the solvent removed by evaporation to yield the final product AzPMA.

In another preferred method, surface-initiated polymerization using two types of monomers, HPMA and AzPMA, was performed using the following procedure: The glass substrate was coated with a thiol moiety by submersing it in 20% (3-Mercaptopropyl) trimethoxysilane in acetone for 60 min. The glass slide was rinsed with acetone twice, and subsequently rinsed with millipore water. The slide was transferred to a schlenk flask, and mixed with a solution of HPMA: 4-cyanopentanoic acid dithiobenzoate: 4,4-azobis(4-cyanovaleric acid)=800:3:1 in methanol. In this example, HPMA 71.59 mg (0.5 mmol) should be dissolved in 0.5 ml methanol with 0.5238 mg of 4-cyanopentanoic acid dithiobenzoate and 0.175 mg of 4,4-azobis(4-cyanovaleric acid). The sample with the solution was subsequently subjected to three-freeze-pump-thaw cycles. The polymerization was performed at 70° C. for 15-20 hours. 71.59 mg AzPMA was added to the flask, and the reaction was carried out for an additional 5 hours. After the reaction, the reacted slides were exposed to ethyl acetate 150 ml, and then to the mixture of methanol and ethyl acetate. The final glass slides had a polymer linker that contained p(HPMA)-p(AzPMA) with 100-200 mononers. (See, e.g., FIG. 18).

The length of PEG linkers can be varied. For example, in this example linkers in the 10-200 nm range were used. Mixing with a small amount of short PEG linkers is recommended when longer PEG linkers are used as the short linkers helps maintain the structural integrity of the longer linkers when attached to a surface.

### Example 2

### Conjugation of Linkers on the Surface and the OS Modulating Molecules

Any specific chemistry can be used to make a chemical bond between a linker and a QS modulating molecule. For example, a bioorthogonal reaction can be used. This reaction is highly selective and has no side reactions. The chemistry is biocompatible and thus not toxic to living organisms, and the fast kinetic reactions make this process especially convenient. Many reported bioorthogonal reactions are known in the art and can be used to conjugate linkers to QS-modulating molecules, such as Staudinger Ligation and/or click chemistry. Additional reactions that are known in the art and that can be used include, but are not limited to: nitrone dipole cycloaddition, norbornene cycloaddition, tetrazine ligation, and/or quadricyclane ligation.

For example, we used the click chemistry method as one example. (See, e.g., FIGS. 14-18). Click chemistry enables covalent bond formation between molecule A with Azide and molecule B with alkyne. Click chemistry uses Cu catalysts to form triazoles by cycloaddition. Here, molecule A was the PEG linker, which has an azide at one end. Molecule B was the QS autoinducer (i.e., AIP for S. aureus), which had an alkyne group attached. Other methods are also possible, for example, the PEG linker can have an alkyne group attached, and the QS modulating molecules can possess azide groups at one end.

First, the alkyne was attached to the QS autoinducer AIP (autoinducing peptide from S. aureus). All amino acid derivatives and resins were purchased from Novabiochem (San Diego, Calif.). All other chemical reagents were purchased from Sigma (St. Louis, Mo.). Analytical gradient reverse-phase high-performance liquid chromatography (HPLC) was performed on a Hewlett-Packard 1100 series instrument. Analytical HPLC was performed on a Vydac C18 column (particle size 5 μm, inner diameter 4.6 mm, length 150 mm) at a flow rate of 1 mL/min. Semi-preparative HPLC was performed routinely on the Vydac C18 column at a flow rate of 4 mL/min. All runs used linear gradients of 0.1% aqueous TFA (solvent A) vs 90% acetonitrile plus 0.1% TFA (solvent B). Mass spectrometry was performed on a Sciex API-100 single quadrupole electrospray mass spectrometer.

Autoinducer peptides (AIPs) were chemically synthesized as described in using standard solid-phase approaches. See, for example, George E A, Novick R P, Muir T W. Cyclic peptide inhibitors of staphylococcal virulence prepared by Fmoc-based thiolactone peptide synthesis. J Am Chem Soc. 2008;130(14): 4914-24. In particular, MBHA resin was preloaded with (tert-butoxycarbonyl) aminoacyl-3-mercaptopro-pionamide. Boc-protected amino acids were coupled with HBTU in DMF, and the deprotection was performed with neat TFA. HF-cleavage was used for releasing of the thioester and the global deprotection. The crude peptide product was precipitated and washed with chilled ethanol. After dissolving the peptide in 50% CH₃CN/50% water/0.1% TFA, MBHA resin was removed through filtration. The crude peptide was purified with semi-preparative RP-HPLC and lyophilized. Cyclization was done by dissolving the peptide in 50% CH₃CN/50% water and 0.1 M phosphate buffer at pH 7 and incubating at room temperature for 2 hours. After another semi-preparative RP-HPLC purification, the product was characterized with analytical RP-HPLC and mass spectrometry.

Next, an azide group attached to one end of the PEG linker that is fixed to the glass surface will react with the alkyne on the modified AIP (QS molecule) in the presence of Cu. Specifically, 1 mM Copper (II) Sulfate in water, 50 mM ascorbic acid and 10 μM alkyne AIP were used to treat the linker-attached substrates for 30 min-60 min at room temperature. Blowing nitrogen air and injection of water were used to remove unbound chemicals. Any autoinducers as described herein (agonist or antagonist) can be used for conjugation to the surfaces. As a proof of principle, we used S. aureus autoinducer (AIP) peptides, but the chemical reaction is identical for other QS molecules.

In this Example, Cu was used as a catalyst. There are also Cu-free ways to conduct the same chemical reaction which are well known in the art.

### Example 3

### Methods for Measuring QS, Biofilm Production, Biofilm Streamer Production and/or Virulence Factor Production

Methods for measuring QS, biofilm production, biofilm streamer production and/or virulence factor production have been reported in the literature and are herein incorporated by reference in their entirety. Kim MK et al. “Local and global consequences of/low on bacterial quorum sensing,” Nature Microbiology 1:15005 2016; Kim MK et al. “Filaments in curved streamlines: Rapid formation of Staphylococcus aureus biofilm streamers,” New J Phys. 2014 Jun 26;16(6):065024; Ng WL, et al., “Broad spectrum pro-quorum-sensing molecules as inhibitors of virulence in vibrios,” PLoS Pathog. 2012;8(6); and O′Loughlin CT, “A Quorum-Sensing Inhibitor Blocks Pseudomonas Aeruginosa Virulence And Biofilm Formation,” PNAS (2013) Oct 29;110(44):17981-6. Compositions of the invention can be tested in any of these published protocols.

### Example 4

### Methods for Measuring QS, Biofilm Production, Biofilm Streamer Production and/or Virulence Factor Production

QS Measurements

QS measurements at the transcriptional level were assessed using promoter fusion analysis. Promoters driving genes responsible for QS (e.g., in the case of P. aeruginosa, the lasl and rhll promoters were used. In the case of V. cholerae, the qrr4 and/or luxC promoters were used. In the case of S. aureus, the agrP3 promoters were used to measure QS activities. In all cases, the promoters were fused to genes encoding fluorescent proteins, luciferase, or the beta-lactamase enzyme, and/or an equivalent which can be quantitatively measured temporally and spatially using a microscopy or a spectrometer. Other promoters and/or reporter proteins could readily be used.

QS phenotypes are diverse, but in the context of healthcare settings, measuring pathogenic traits that are regulated by QS systems, such as virulence factor production and biofilm formation, are of interest. The following example assays may be used to quantitatively measure such traits.

Virulence Factor Production Measurements

Virulence factor production at the transcriptional level is assessed using promoter fusion analysis. Promoters driving genes responsible for virulence factors, e.g., in the case of P. aeruginosa, the lasAB and rhlAB promoters were used. In the case of V. cholerae, the ctxAB, toxT and hapA promoters were used. In the case of S. aureus, the hldBC and clfB promoters were used. In all cases, the promoters were fused to genes encoding fluorescent proteins, luciferase, or the beta-lactamase enzyme, or an equivalent which can be quantitatively measured temporally and spatially using microscopy or a spectrometer. The actual virulence factor (toxin, enzymes, etc.) can also be measured directly. Specifically, one can measure or verify the results from promoter-reporter fusions using enzyme-linked immunosorbent assay (ELISA) techniques, in which toxins from a sample are transferred to a membrane, and subsequently, antibodies that recognize the specific toxin are introduced. The antibodies are usually linked to an enzyme or a fluorophore that can be quantitatively measured.

Biofilm Production Analysis

One can measure the amount of biofilms formed using cells carrying a constitutively expressed fluorescent protein. Microscopy can be used to measure the 3D volumes or biomass.

Biofilms can also be measured using a conventional method. There are many commercially available stains that specifically bind to components of biofilms, such as the polysaccharide matrix and/or extracellular DNA. Subsequently, using microscopy, the amount of biomass can be quantified. Biofilms can also be measured in a commonly used microtiter plate assay and crystal violet staining.

### Example 5

### Construction of Bacterial Strains and Plasmids

The strains and plasmids used are listed in Table 4. Staphylococcus aureus strains include RN4220, RN9011, RN6390b, RN6911, and RN6607. Plasmids include pJL1111 and pRN7062. S. aureus strains MK121¹⁸ (RN6390b carrying pMK021; agrP3-gfpmut2, sarAP1-mkate2) and S. aureus MRSA strain MK131¹⁸ (BAA1680 carrying pMK021) were also used.

TABLE 4

| | | |
|---|---|---|
| Strain/plasmid | Genotype/description | References/Sources |

E. coli

| | | |
|---|---|---|
| DH5α | Cloning strain, F′ proA⁺B⁺ lacI^{q} Δ(lacZ)M15 zzf::Tn10 (Tet^{R})/ | NEB |
| | fhuA2Δ(argF-lacZ)U169 phoA glnV44 Φ80Δ(lacZ)M15 gyrA96 | |
| | recA1 relA1 endA1 thi-1 hsdR17 | |

S. aureus

| | | |
|---|---|---|
| RN4220 | Restriction-deficient mutant of strain 8325-4, transformable | S1 |
| | cloning host | |
| RN9011 | RN4220 containing pRN7023 (SaPI-1 integrase) | S2 |
| RN6390b | Standard agr-I wild-type, derivative of NTCT8325-4 | S3 |
| RN6911 | RN6390b replacing agrBDCA and RNAIII with tetM (ΔagrBDCA | S4 |
| | ΔRNAIII) | |
| RN6607 | Standard agr-II wild-type | S6 |
| MK231 | RN6911 SaPI-1attC::pMK031 (sarAP1-mturquoise2 in the | This study |
| | genome) | |
| MK232 | RN6911 SaPI-1attC::pMK032 (sarAP1-gfpmut2 in the genome) | This study |
| MK233 | RN6911 SaPI-1attC::pMK033 (sarAP1-mko in the genome) | This study |
| MK241 | MK231 containing pMK051 | This study |
| MK242 | MK232 containing pMK051 | This study |
| MK243 | MK233 containing pMK051 | This study |
| MK244 | MK232 containing pMK014 | This study |
| MK245 | MK232 containing pMK004 | This study |
| MK260 | RN6911 SaPI-1attC::pMK060 (agrP2-agrCA in the genome) | This study |
| MK261 | MK264 containing pMK004 | This study |
| MK264 | RN6911 SaPI-1attC::pMK064 (agrP2-agrCA, agrP3-mkate2 in | This study |
| | the genome) | |
| MK265 | MK264 containing pMK012 | This study |
| MK121 | RN6390b containing pMK021; agrP3-gfpmut2, sarAP1-mkate2 | S5 |
| | Note that pMK021 does not contain agrP 2-agrCA. | |
| MK131 | Methicillin resistant strain (MRSA), clinical isolate from human | S5 |
| | skin, agr group I strain containing pMK021; agrP3-gfpmut2, | |
| | sarAP1-mkate2 | |
| MK125 | RN6390b containing pMK013; sarAP1-mko | This study |
| MK126 | RN6607 containing pMK013; sarAP1-mko | This study |

Plasmids

| | | |
|---|---|---|
| pMK004 | pCN54 (Erm^{r}) containing agrP3-mkate2 | S5 |
| pMK011 | pCN54 (Erm^{r}) containing sarAP1-mturquoise2 | This study |
| pMK012 | pCN54 (Erm^{r}) containing sarAP1-gfpmut2 | This study |
| pMK013 | pCN54 (Erm^{r}) containing sarAP1-mko | This study |
| pMK014 | pCN54 (Erm^{r}) containing sarAP1-mkate2 | S5 |
| pRN7062 | pCN54 (Erm^{r}) containing agrP2-agrCA, agrP3-lacZ | S6 |
| pMK051 | pCN54 (Erm^{r}) containing agrP2-agrCA, agrP3-mkate2 | This study |
| pJC1111 | SaPI-1 attS suicide vector containing cadmium resistant (cadCA) | S7 |
| pMK031 | pJC1111 (Cad^{r}) containing sarAP1-mturquoise2 | This study |
| pMK032 | pJC1111 (Cad^{r}) containing sarAP1-gfpmut2 | This study |
| pMK033 | pJC1111 (Cad^{r}) containing sarAP1-mko | This study |
| pMK060 | pJC1111 (Cad^{r}) containing agrP2-agrCA | This study |
| pMK064 | pJC1111 (Cad^{r}) containing agrP2-agrCA, agrP3-mkate2 | This study |

DNA polymerase, dNTPs, and restriction enzymes were purchased from New England Biolabs (NEB, Ipswich, Mass.). DNA extraction and purification kits were acquired from Qiagen (Valencia, Calif.). DNA oligonucleotides were purchased from Integrated DNA Technologies (Coralville, Iowa). Sequences of plasmids were verified by Genewiz (South Plainfield, N.J.).

Plasmids carrying constitutively expressed fluorescent fusions were constructed by replacing the mkate2 gene from pMK014 (sarAP1-mkate2) with genes encoding different fluorescent proteins (gfpmut2, mturquoise2, and mko). To make these plasmids, the gfpmut2 gene was amplified by PCR from pMK021¹⁸ using primers MKF013/MKR013, the mturquoise2 gene⁵¹ was amplified by PCR from pDP428 using primers MKF011/MKR011 and the mko gene was amplified by PCR from pCN005⁵² using primers MKF014/MKR014. mkate2 was replaced with an amplified gene by overlap extension PCR cloning. These plasmids were called pMK012 (sarAP1-gfpmut2), pMK011 (sarAP1-mturquoise2), and pMK013 (sarAP1-mko).

The constitutively expressed reporter fusions were integrated into the S. aureus chromosome using a site-specific integration suicide vector, pJC1111, carrying a cadmium resistance cassette and a SaPI-1 attS sequence which integrates into the S. aureus chromosomal attachment site (attC) of pathogenicity island 1 (SaPI-1)⁵³. This plasmid was integrated in single copy and maintained stably⁵³. pJC1111 was digested using restriction enzymes NarI/SphI. The sarAP1-gfpmut2 gene was amplified by PCR from pMK012 using primers MKF031/MKR031 followed by digestion with NarI/SphI and ligation into digested pJC1111. This plasmid was called pMK032 (sarAP1-gfpmut2 in the suicide vector). The same procedure was used for other fluorescent genes, pMK031 (sarAP1-mturquoise2 in the suicide vector), and pMK033 (sarAP1-mko in the suicide vector). The plasmids were introduced into Escherichia coli DH5α using chemical transformation (New England Biolabs, Ipswich, Mass.) followed by selection with ampicillin. The plasmids were purified from E. coli, introduced by electroporation into S. aureus strain RN9011, which expresses the SaPI-1 integrase, and colonies containing the fusions integrated into the chromosome were selected with cadmium. Subsequently, the chromosomal integrants were transduced into S. aureus strain RN6911 using standard phage transduction techniques with phage 80α. These strains were called MK232 (sarAP1-gfpmut2 in the genome), MK231 (sarAP1-mturquoise2 in the genome), and MK233 (sarAP1-mko in the genome). Primers are shown in Table 5.

TABLE 5

| Primers | Sequence (5′-3′) |
|---|---|
| MKF011 | TCGTTAACTAATTAATTTAAGAAGGAGATATACATATGGTAT |
| (SEQ ID | CAAAAGGGGAAGAGTTG |
| NO: 1) | |
| MKR011 | TTAGAATAGGCGCGCCTTATTTGTACAGTTCGTCCATGCC |
| (SEQ ID | |
| NO: 2) | |
| MKF013 | TCGTTAACTAATTAATTTAAGAAGGAGATATACATATGAGTA |
| (SEQ ID | AAGGAGAAGAACTTTTCACT |
| NO: 3) | |
| MKR013 | TTAGAATAGGCGCGCCTTATTATTTGTATAGTTCATCCATGC |
| (SEQ ID | CATG |
| NO: 4) | |
| MKF014 | TCGTTAACTAATTAATTTAAGAAGGAGATATACATATGGTGA |
| (SEQ ID | GTGTGATTAAACCAGAG |
| NO: 5) | |
| MKR014 | TTAGAATAGGCGCGCCTTAGGAATGAGCTACTGCATCTTCTA |
| (SEQ ID | |
| NO: 6) | |
| MKF031 | TATAATAGCATGCACATAACACCAAAAAGAAGAAGGTGC |
| (SEQ ID | |
| NO: 7) | |
| MKR031 | CCGCAAAGGCGCCTGTCACTTTGCTTGATATATGAG |
| (SEQ ID | |
| NO: 8) | |
| MKF032 | AATACGCCGTTAACTGACTTTATTATCTTATTATATTTTTTT |
| (SEQ ID | AACGTTTCTCACCGATGC |
| NO: 9) | |
| MKR032 | GGAGGGGCTCACGACCATACTTACATGTCAACGATAATACAA |
| (SEQ ID | AATATAATACAAAATATA |
| NO: 10) | |
| MKF033 | TTGAATACGCCGTTAACTGACTTTATTATCTTATTATATTTT |
| (SEQ ID | TTTAACGTTTCTCACCGA |
| NO: 11) | |
| MKR033 | GGAGGGGCTCACGACCATACTTA |
| (SEQ ID | |
| NO: 12) | |

A plasmid carrying a transcriptional fusion to monitor S. aureus Agr QS activity was constructed by replacing the lacZ gene from vector pRN7062⁵⁴ (agrP3-lacZ) with the mkate2 gene. pRN7062 also harbored the genes encoding the Agr QS detection components agrCA under their native agrP2 promoter but driven in the opposite direction. To make this plasmid, the lacZ gene was removed from pRN7062 by digestion with EcoRI/NarI. The mkate2 gene was obtained from pMK014 by EcoRI/NarI digestion. The digested mkate2 gene was ligated into digested pRN7062. This plasmid was called pMK051 (agrP2-agrCA, agrP3-mkate2). This construct was first introduced into E. coli, purified, and subsequently introduced into S. aureus strain RN4220 using selection with erythromycin. Subsequently, using phage transduction, the plasmid was introduced into S. aureus strains MK232, MK231, and MK233. The resultant strains were called MK242 (sarAP1-gfpmut2 in the genome and pMK051), MK241 (sarAP1-mtor2 in the genome and pMK051), MK243 (sarAP1-mko in the genome and pMK051). To construct the S. aureus AagrBDCA strain harboring agrP3-mkate2 in a plasmid, the vector pMK004¹⁸ (agrP3-mkate2) was introduced into S. aureus strain MK232 (sarAP1-gfpmut2 in the genome of S. aureus AagrBDCA), leading to strain MK245.

Control strains were constructed to study heterogeneity of the Agr QS response. The first control strain had the agrP2-agrCA and agrP3-mkate2 genes inserted into the genome of RN6911, and harbored sarAP1-gfpmut2 in a plasmid. To make this strain, the agrP2-agrCA, and agrP3-mkate2 genes from pMK051 were amplified using primers MKF032/MKR032, and this fragment was inserted into the suicide vector pJC1111 by overlap extension PCR cloning. This plasmid was called pMK064 (agrP2-agrCA, agrP3-mkate2 in the suicide vector). The gene was integrated into the S. aureus strain RN6911 chromosome as described above. This strain was called MK264 (agrP2-agrCA, agrP3-mkate2 in the genome). The vector pMK012 (sarAP1-gfpmut2) was introduced into MK264, leading to strain MK265 (agrP2-agrCA, agrP3-mkate2 in the genome and pMK012). The second control strain was constructed by introducing pMK014 (sarAP1-mkate2) into strain MK232, leading to MK244 (sarAP1-gfpmut2 in the genome and pMK014). The third control strain had the agrP2-agrCA gene inserted into the genome of RN6911, and harbored agrP3-mkate2 in a plasmid. To construct this strain, the agrP2-agrCA gene from pMK051 was amplified using primers MKF033/MKR033, this fragment was inserted into the suicide vector pJC1111 by overlap extension PCR cloning. This plasmid was called pMK060 (agrP2-agrCA in the suicide vector). The gene was integrated into the S. aureus strain RN6911 chromosome as described above. This strain was called MK260 (agrP2-agrCA in the genome). The vector pMK004¹⁸ (agrP3-mkate2) was introduced into MK260, leading to strain MK261 (agrP2-agrCA in the genome and pMK004). Finally, a constitutively expressed mKO fluorescent reporter (sarAP1-mko in pMK013) into wild-type S. aureus agr-I (strain RN6390b) and wild-type S. aureus agr-II (strain RN6607) was used to measure the number of cells in biofilms on surfaces.

### Example 6

### Growth Conditions

S. aureus RN6911 derivatives were grown overnight at 37° C. with shaking in Tryptic Soy Broth (TSB; Difco, Franklin Lakes, N.J.) with 10 μg/ml tetracycline and 10 μg/ml erythromycin to maintain plasmids, back-diluted 1:200, and re-grown for 3 h (to OD₆₀₀˜0.05-0.1). S. aureus MK121, MK131, MK125, and MK126 were grown overnight at 37° C. with shaking in TSB with 10 μg/ml erythromycin, back-diluted 1:2000, and re-grown for 3 h (to OD₆₀₀˜0.05-0.1).

### Example 7

### Synthesis of AIP-I, AIP-II, and TrAIP-II Derivatives

AIP-I, AIP-II, and TrAIP-II derivatives were synthesized using a combined solid-phase/solution-phase approach. Linear peptide a-thioester precursors were generated using Fmoc-solid phase peptide synthesis employing a hydrazine linker system. The peptides were then cyclized in solution to create the thiolactone macrocyclic.

### Example 8

### Fluorescence Reporter Assay

Transcription from fluorescence reporter genes was measured in S. aureus strain MK242. Overnight cultures were diluted 1:200 into fresh TSB with 10 μg/ml tetracycline and 10 μg/ml erythromycin, re-grown, and 90 μl of these cultures were distributed into wells of 96 well plates (MatTek, Ashland, Mass.), followed by addition of 10 μl of AIP-I and/or TrAIP-II and/or derivatives. Subsequently, 50 μl of mineral oil was added (Sigma, St. Louis, Mo.) to prevent evaporation. Using a Synergy 2 plate reader (Biotek, Winooski, Vt.), GFPmut2 and mKate2 levels were measured at 484 nm/528 nm and 588 nm/633 nm, respectively. Measurements were conducted with 15 min intervals at 37° C. with shaking. This assay was called “Solution Assay”.

### Example 9

### Surface Fabrication

Surface-PEG₁₀₀₀₀-azide, Surface-PEG₄₀₀-azide, Surface-PEG₁₀₀₀₀, and Surface-PEG₄₀₀ were fabricated as follows (see FIG. 9a). Glass slides (36×60 mm², Ted Pella, Redding, Calif.) were boiled for 2 h in 5% sodium peroxodisulfate (Sigma) at 50° C. and washed twice with Millipore water. The washed slides were submerged in piranha etch solution (3:1 H₂SO_{4:}H₂O_{2,} Fisher Scientific, Hampton, N.H.) for 6 h. Next, the slides were washed twice with Millipore water followed by rinsing with acetone (Sigma) three times to remove any trace of water. The hydroxylated glass substrate was incubated in 25% (3-mercaptopropyl) trimethoxysilane (Sigma) in acetone for 60 min to decorate the surface with thiol (—SH) functional groups. The thiol-decorated surface was rinsed with acetone and then Millipore water. After drying the substrates, the surfaces were incubated with a polymer-containing solution such as maleimide-PEG₁₀₀₀₀-azide, maleimide-PEG₄₀₀-azide, maleimide-PEG₁₀₀₀₀, or maleimide-PEG-₄₀₀ (100 g/L, Nanocs, New York, N.Y.) in 0.9 M sodium sulfate (Sigma) and 10 mM pH=7.2 Tris-HCl buffer for 12 h to undergo the thiol-maleimide addition reaction³⁴. The substrates were rinsed with Millipore water to yield Surface-PEG₁₀₀₀₀-azide, Surface-PEG₄₀₀-azide, Surface-PEG₁₀₀₀₀, and Surface-PEG_{400,} respectively. To fabricate the PDMS-based Surface-PEG₁₀₀₀₀-azide, glass slides were spin-coated with a PDMS mixture from the Sylgard 184 elastomer kit (Dow Corning, Midland, Mich.) at 2000 rpm for 30 sec. PDMS-coated surfaces were allowed to solidify, and then submerged in H₂O:H₂O₂:HCl (5:1:1) for 1 h. Next, the slides were washed twice with Millipore water followed by rinsing with acetone (Sigma) three times to remove any trace of water, rendering the hydroxylated PDMS-based substrate. The remainder of the procedures are identical to those described above for glass surfaces. To fabricate the gold-based Surface-PEG5000-azide, coverslips coated with 10 nm thickness of gold (Amsbio, Cambridge, Mass.) were washed three times with acetone, and subsequently washed with isopropanol for 15 min. After drying the gold-coated substrates, thiol-PEG₅₀₀₀-azide (100 g/L, Nanocs) in 0.9 M sodium sulfate was added to the surface for 12 h to undergo the thiol-gold reaction. Following the reaction, the surfaces were washed thoroughly with Millipore water.

The azides decorating the surface-attached PEG₄₀₀ and PEG₁₀₀₀₀ were subjected to the click reaction to link molecules carrying alkyne groups, such as Alkyne-AIP-I, Alkyne-AIP-II, Alkyne-TrAIP-II, and Alkyne-dye. The solution for the click reaction contained: 1 mM CuSO₄, 50 mM ascorbic acid, 5 mM Tris (3-hydroxypropyltriazolylmethyl)amine, and 100 μM alkyne molecule in 100 mM phosphate buffer (pH=7). Surface-PEG10000-azide and Surface-PEG₄₀₀-azide were incubated with the click solution containing the alkyne molecules for 3 h. After rinsing the substrates three times with dimethyl sulfoxide (50% in water, Sigma) to remove any unreacted chemicals, the surfaces had become linked by the triazole rings to the AIP-I, AIP-II, TrAIP-II, or the dyes.

To investigate long-term surface stability, the Surface-PEG₁₀₀₀₀-triazole-AIP-I and the Surface-PEG₁₀₀₀₀-triazole-dye were stored at 4° C. for 40 days in a wet container covered with aluminum foil to prevent desiccation and exposure to light.

### Example 10

### Surface Characterization

The modified surfaces were characterized using a Fourier transform infrared (FTIR) spectrometer, fluorophore labeling, or atomic force microscopy (AFM) as follows: An FTIR spectra for the Surface-PEG₁₀₀₀₀-azide was obtained as an averaged signal by scanning the sample 128 times at 4 cm⁻¹ resolution using a Thermo Nicolet Nexus 670 FTIR spectrometer (Thermo Electron Corp., Waltham, Mass.) equipped with a liquid nitrogen-cooled MCT/A detector (FIG. 9b). Prior to taking the spectra of the Surface-PEG₁₀₀₀₀-azide, the unmodified glass surface was scanned to provide the background spectrum. Background and sample measurements were taken only after the sample chamber was purged sufficiently with dry nitrogen to reduce the levels of carbon dioxide and water vapor.

To characterize the Surface-PEG₁₀₀₀₀-azide, Alexa Fluor 555 dye functionalized with an alkyne moiety (Thermo Fisher, Mass.) was used. The Surface-PEG₁₀₀₀₀ was used as the negative control. The surfaces were treated with the click solution containing the Alexa Fluor 555 alkyne dye. After washing away the unreacted dyes, the surfaces were imaged by confocal microscopy (FIG. 9c). Surface-PEG₄₀₀-azide (FIG. 11a), gold-based Surface-PEG₅₀₀₀-azide, and PDMS-based Surface-PEG₁₀₀₀₀-azide (FIG. 13c) were characterized using the identical procedure.

AFM was used to measure the heights of the polymer brushes on the Surface-PEG₁₀₀₀₀-azide and the Surface-PEG₄₀₀-azide. The surfaces were allowed to dry via evaporation in air. AFM topographical images of the boundaries between the PEG-attached regions and the unmodified regions were obtained in air using a Bruker Dimension Icon AFM (Bruker Biosciences, Billerica, Mass.) equipped with AFM tips (Bruker Biosciences) in Scanasyst-air peak force tapping mode. The scan regions were 2×2 μm². The heights of surface-attached polymers were measured as the difference between the unmodified region and the PEG-attached region. Several regions from two independently fabricated surfaces were scanned.

### Example 11

### QS Gene Expression Analyses on Surfaces

The chemically modified surfaces were bonded to microfluidic chambers (400 μm×100 μm×2 cm) using an epoxy glue (Fisher Scientific). The assembled chambers were inoculated with the S. aureus agr-I reporter strain cultures, and cells were allowed to settle onto surfaces for 10 min, after which sterile M9 medium containing 10 μg/ml erythromycin, 0.5% glucose, 0.5% casamino acids, 5 mM NaCl, 50 mM MgSO_{4,} and 0.1 mM CaCl₂ was flowed steadily into the devices for 30 min to remove planktonic cells. After this step, chambers were placed on the microscope and the fluorescent reporter output measured. In experiments containing autoinducer or antagonist in solution, molecules were added in the wash medium. In experiments containing human blood plasma (Biological Specialty Corp, Colmar, Pa.), 100% blood plasma solution was initially flowed into the chambers containing the chemically modified surfaces for 10 min. Subsequently, S. aureus agr-I reporter cells were seeded for 10 min, and then 20% or 50% blood plasma diluted in the above medium was flowed into the chambers for 30 min to remove the planktonic cells. After this step, chambers were placed on the microscope and the fluorescent reporter output measured.

### Example 12

### Biofilm Analyses on Surfaces

Chambers containing modified surfaces were seeded with wild-type S. aureus strains harboring constitutively expressed mko on a plasmid, and the cells were allowed to settle onto the surfaces for 10 min, after which sterile TSB containing 3% NaCl and 10 μg/ml erythromycin was flowed steadily into the devices for 8 h. This procedure was sufficient to produce three-dimensional S. aureus biofilms of 10-30 μm thickness. Flow was terminated for 10 h, and subsequently, planktonic cells were removed by steady flow for 10 min. The remaining biofilms were measured by confocal microscopy. Cells in biofilms covering a surface area of 225×225 μm² were imaged at six different regions for each surface type in n=3 independent experiments.

### Example 13

### Microscopy and Imaging

Imaging was performed using a Nikon Eclipse Ti inverted microscope (Melville, N.Y.) fitted with a Yokogawa CSU X-1 confocal spinning disk scanning unit (Biovision Technologies, Exton, Pa.) and DU-897 X-9351 camera (Andor, Concord, Mass.). Laser lines at 445, 488, 543, and 592 nm were used to excite the mTurquoise2, GFPmut2, mKO, and mKate2 fluorescent proteins, respectively. Laser lines at 488, 543, and 592 nm were used to excite Alexa Fluor 488 fluorophore, Alexa Fluor 555 fluorophore, and Alexa Fluor 594 fluorophore, respectively. In order to obtain single-cell resolution, both a 100× oil objective with N.A. 1.4 (Nikon, Melville, N.Y.) and a 1.5× lens placed between the CSU X-1 and the Nikon microscope side port were used. Consequently, the magnification of 0.1 μm per pixel in the XY plane was obtained. For single-cell analysis, custom code was written in Matlab. Briefly, the area of an individual cell was recognized and segmented using a watershed-based algorithm. In this process, cells were removed if they were on the edge of the image or if they were smaller than 30% of the average cell size, suggesting that they were out of focus. In the area of an individual cell, both the constitutive GFPmut2 fluorescence and the QS controlled mKate2 fluorescence were measured, subtracted from background signals and summed. The normalized QS output was calculated as the QS controlled mKate2 intensity divided by the constitutively expressed GFPmut2 intensity in individual cells. In each experiment, images of many regions on the surfaces were taken to include 1000-4000 individual cells. Each replicate was performed using independent bacterial cultures and independent surfaces at room temperature. Identical procedures were performed for the strains harboring different constitutive fluorescent proteins such as mTurquoise2 and mKO (FIG. 5b). Custom code was used to count the cells in the biofilms. Each image was segmented in the z-plane and assessed independently (FIGS. 4d and 13c).

### Example 14

### Quantitation of Surface Coverage Density and Single-Molecule Microscopy

The coverage density of “clicked” azides on the Surface-PEG₁₀₀₀₀-azide was quantified based on fluorophore labeling. The alkyne-dye, the Alkyne-AIP-I, and the Alkyne-TrAIP-II were assumed to have identical reactivity in the click reaction, rendering the same surface coverage density. Thus, surface coverage density can be quantified using fluorophore labeling as a proxy for surface-attached QS compounds. In the fluorophore labeling assay, the surface-attached azides underwent the click reaction with 100 μM Alexa Fluor 555 alkyne fluorophore. After conjugation, the total intensity of fluorescence from the surface was measured (at 10% laser power), subtracted from the background signal, and divided by the average single-molecule intensity. The average single-molecule intensity was obtained by measuring the intensity of single Alexa Fluor 555 molecules as follows: the Surface-PEG₁₀₀₀₀-azide was treated with 0.1−10 nM Alexa Fluor 555 alkyne fluorophore, and this low concentration of alkyne-dye produced individually discernable fluorescent spots (20-300 in a 51×51 μm²) (FIG. 11d (i)). The individual fluorescent spots were subjected to photobleaching, and displayed stepwise decreases in intensity. Background subtracted intensities of a few thousand spots across 5 independent dye-linked surfaces were measured and analyzed using custom Matlab code. Briefly, the obtained images were smoothed and filtered to produce a zero-based image in which bright fluorescent spots were located with pixel level accuracy by a peak-finding algorithm. With the high spatial resolution of the microscope (100 nm/pixel), the intensity of each spot was measured from an 8 pixel×8 pixel region centered at each of these peaks. The integrated fluorescence emission within each region was measured over consecutive frames while exposing the sample to high intensity illumination (at 100% laser power with 500 msec exposure time) to promote photobleaching. The photobleaching intensity trace revealed the number of fluorophores present in that spot, and the size of these intensity steps yielded the intensity of emission from the fluorophores in the spot. Most fluorescent spots decreased in one step following bleaching (FIG. 11d (ii)). A small number of spots bleached in two steps (FIG. 11d (ii)). By fitting the bleaching step sizes for over 3,600 spots with a normal distribution (FIG. 11d (iii)), we were able to arrive at the average single-molecule intensity (7,000 (AU)), which was further verified by counting fluorescent spots from the surface. Unlike the reaction of the surface with the low concentration of alkyne-dye, the surface intensity from the typical surface that had reacted with 100 μM alkyne-dye was above the maximum detection limit of the camera when exposed to 100% laser power. Thus, a two-step calibration process was used in which the total intensity of uniform surfaces that had reacted with 1 nM, 5 nM, and 10 nM alkyne-dye at 100% laser power was measured and compared to that obtained with 10% laser power, yielding an average ratio of 49. For the final surface measured at 10% laser power with 500 msec exposure time, the integrated intensity was multipled by 49, and divided by the average single-molecule intensity (7,000 (AU)), to give a surface coverage density.

### Example 15

### Surface-Attached AIP-I Activated OS

Surface-attached compounds were used to positively and negatively manipulate S. aureus Agr QS. Copper-catalyzed azide-alkyne cyclo-addition click chemistry was used to attach active compounds to surfaces³³. Polyethylene glycol (PEG) polymers decorated with azide groups were selected as the surface linkers because PEG is flexible, hydrophilic, and non-bulky.

To make AIP-I amenable to surface attachment, AIP-I containing an alkyne at the N-terminus was synthesized (FIG. 7a and FIG. 8a). This compound was called “Alkyne-AIP-I” (FIG. 2a). Prior to attaching the Alkyne-AIP-I to the surface linkers, the click reaction was carried out between the Alkyne-AIP-I and a surface-free version of the PEG₃₃₀-azide linker to test if the AIP-I derivatives retained their activation capability following the click reaction. The reaction between the alkyne and azide produced a triazole ring linking AIP-I to the PEG₃₃₀ polymer. This compound was called “PEG₃₃₀-triazole-AIP-I” (FIG. 2a (iii), FIG. 7a and FIG. 8b). In solution, AIP-I, Alkyne-AIP-I, and PEG₃₃₀-triazole-AIP-I activated Agr QS in the S. aureus reporter strain with similar efficacy albeit with different potencies (FIG. 2a (iii) and FIG. 2b). The calculated EC₅₀ values were 28 nM (±3) for AIP-I, 190 nM (±40) for Alkyne-AIP-I, and 1.1 μM (±0.20) for PEG₃₃₀-triazole-AIP-I (Table 6). Addition of 2.5 μM TrAIP-II repressed the Agr QS response to all three compounds by a comparable magnitude (FIG. 2b). These results indicate that, analogous to AIP-I, both Alkyne-AIP-I and the PEG₃₃₀-triazole-AIP-I activated QS by targeting the AgrC-I receptor.

Table 6. EC₅₀ values (from n=3 experiments) for AIP-I and its derivatives and IC₅₀ values (from n=4 experiments) for TrAIP-II and its derivatives when AIP-I is present at 100 nM (i.e., the EC₉₅).

TABLE 6

| | | | |
|---|---|---|---|
| EC₅₀ (nM) | AIP-I | 28 | (±3) |
| | Alkyne-AIP-I | 190 | (±40) |
| | PEG₃₃₀-triazole-AIP-I | 1100 | (±200) |
| IC₅₀ (μM) | TrAIP-II | 1.5 | (±0.5) |
| | Alkyne-TrAIP-II | 0.21 | (±0.05) |
| | PEG₃₃₀-triazole-TrAIP-II | 3.1 | (±1.5) |

For the surface modification work, silanization and maleimide-thiol chemistry was used to decorate the surface with PEG₁₀₀₀₀ polymers carrying azide moieties at one end (FIG. 9), this was called the “Surface-PEG₁₀₀₀₀-azide”. Calculations of the height of the PEG brush³⁴ suggested that a PEG₁₀₀₀₀ polymer would have sufficient length to span the peptidoglycan layer and would position the attached AIP-I to interact with the AgrC-I receptor located on the cell membrane. A click reaction was carried out to covalently attach the Alkyne-AIP-I to the Surface-PEG₁₀₀₀₀-azide (FIG. 2a (iv)), generating “Surface-PEG₁₀₀₀₀-triazole-AIP-I”. To examine whether the PEG-triazole-AIP-I moiety remained functional when attached to the surface, the S. aureus reporter strain was provided to the Surface-PEG₁₀₀₀₀-triazole-AIP-I in microfluidic chambers. The Agr QS response was induced (see “Surface Assay”, FIG. 2a (iv) and FIG. 2c). Compared to T=0 h when cells were in the QS-off mode, the response was activated over time and reached an average of 25-fold activation at T=6 h (FIG. 2c,d and 10a). These results suggested that the surface-attached AIP-I was recognized as an autoinducer by the cognate membrane-bound AgrC-I receptor. To confirm this interpretation of surface-tethered AIP-I eliciting the Agr QS response in S. aureus, 2.5 μM TrAIP-II antagonist was provided in solution to the S. aureus cells residing on the AIP-I coated surface. The Agr QS response was repressed (FIG. 2c and FIG. 10b (i)).

To reinforce the above results, it was also shown that S. aureus did not activate a QS response when introduced onto the identical surface lacking the triazole-AIP-I decoration (Surface-PEG₁₀₀₀₀) or that had not undergone the click reaction (Surface-PEG₁₀₀₀₀-azide) (FIG. 2c, FIG. 10b (ii) and (iii)). Furthermore, the reporter strain that was presented to a surface coated with the identical PEG₁₀₀₀₀ polymer attached, via a triazol ring, to a ring-opened version of AIP-I did not elicit a response (see compound 6 in FIG. 7a, FIG. 2c and FIG. 10b (iv); called Surface-PEG₁₀₀₀₀-triazole-Linear-AIP-I). Identical results were obtained using the Surface-PEG₁₀₀₀₀-triazole-AIP-I in which the thioester ring was opened via treatment with 10 mM cysteine (FIG. 10b (v)). Consistent with these results, an S. aureus reporter strain lacking AgrC-I did not respond to the surface-attached AIP-I (FIG. 10b (vi)). Thus, surface-attached AIP-I specifically and reversibly binds to AgrC-I , indicating that it functions as an autoinducer.

### Example 16

### Crucial Features of Surface-Attached OS Molecules

By changing particular features of the tethered molecules for the surface-attached AIP-I to bind AgrC-I and activate Agr QS in S. aureus, it could be determined which features of these molecules were responsible for activity. Presumably, AgrC-I diffuses freely in the membrane³⁵. In S. aureus, the plasma membrane is covered by a 15-30 nm peptidoglycan layer with 4-5 nm diameter pores³⁶. Peptidoglycan is considered to be an elastic mesh-network that can expand, contract, and tolerate transport of globular molecules of up to 100 kDa³⁶.

Initially, it was determined how altering the length of the polymer attaching the AIP-I to the surface influenced the S. aureus Agr QS response. AIP-I was attached with two different surface linkers: Surface-PEG₁₀₀₀₀-azide and Surface-PEG₄₀₀-azide. Using atomic force microscopy, the heights of the linkers were measured to be 26.5 nm (±2.5) and 1.9 nm (±0.2), respectively. The calculated Flory radius of PEG₁₀₀₀₀ in solution was previously determined to be ˜10 nm³⁴, indicating that the surface-attached PEG₁₀₀₀₀ exists in an extended configuration due to local crowding. This configuration suggests that the terminal functional groups are exposed and thus could properly position attached AIP-I molecules to interact with the AgrC-I receptors. Indeed, the azide moieties on both polymers were amenable to the click reaction using alkyne-functionalized dyes (Surface-PEG₁₀₀₀₀-azide, FIG. 9c and Surface-PEG₄₀₀-azide, FIG. 11a). Finally, the Alkyne-AIP-I was attached to both surfaces, and it was determined that the Surface-PEG₁₀₀₀₀-triazole-AIP-I activated the S. aureus Agr QS response whereas the Surface-PEG₄₀₀-triazole-AIP-I did not (FIG. 11b,c). It was thought that the latter result was due to a geometrical restriction related to the PEG₄₀₀ length, and that this shorter linker did not appropriately position the AIP-I to access AgrC-I receptors on the plasma membrane. As a control, it was shown that the S. aureus reporter strain activated Agr QS on the Surface-PEG₄₀₀-triazole-AIP-I if it was also provided with 50 nM AIP-I in solution (FIG. 11b and FIG. 11c (iii)).

To examine how surface coverage density of PEG₁₀₀₀₀-triazole-AIP-I affects activation of AgrC-I-directed QS, the number of reacted azides in a unit area on the Surface-PEG₁₀₀₀₀-azide following the click reaction was measured. To do this, a fluorescent dye harboring an alkyne was joined to the Surface-PEG₁₀₀₀₀-azide (FIG. 9c (ii)) using click chemistry. The intensity of fluorescence from the surface is directly proportional to the number of reacted azide moieties. An average intensity was calculated for each dye molecule (FIG. 11d), which enabled calculation of the coverage density of reacted azides by dividing the total integrated intensity in a unit surface area by the average single-molecule intensity. The coverage density of the clicked azide was calculated to be 2.1×10⁴ μm⁻² (±0.11). The alkyne dye and the Alkyne-AIP-I were assumed to have identical reactivity in the click reaction³³, thus rendering the same surface coverage density, which is sufficient to stimulate the S. aureus Agr QS response (FIG. 11e). The surface coverage density of active AIP-I was reduced by mixing the Alkyne-AIP-I and the Alkyne-Linear-AIP-I (i.e., the inactive counterpart) at different ratios prior to attachment to the surface. It was found that Agr QS activation depended on the surface-attached AIP-I coverage density (FIG. 11e). At a coverage density below 2.1×10² μm⁻², the surface-attached AIP-I did not elicit the QS response in S. aureus, and above a coverage density of 1.6×10⁴ μm⁻² the response was saturated (FIG. 11e). Together, these results demonstrated that the length of the PEG polymer coupled with the coverage density of the surface-bound AIP-I molecules were key factors for AgrC-I-directed QS activation.

### Example 17

### OS was Inhibited by Surface-Attached TrAIP-II

It was investigated whether a QS antagonist, when immobilized on a surface, could interfere with Agr QS signal transduction. A clickable QS antagonist was synthesized, the Alkyne-TrAIP-II (FIG. 7b and FIG. 8c), and attached to the PEG₃₃₀-azide in solution and to the Surface-PEG₁₀₀₀₀-azide (FIG. 3a), rendering PEG₃₃₀-triazole-TrAIP-II and Surface-PEG₁₀₀₀₀-triazole-TrAIP-II, respectively. The products were charctarized for AIP-I (FIG. 8d). In solution, and in the absence of AIP-I, the PEG₃₃₀-triazole-TrAIP-II elicited no Agr QS activation. When AIP-I was supplied at 0.1 μM (i.e., at its EC₉₅), all of the TrAIP-II derivatives (i.e., Alkyne-TrAIP-II and PEG₃₃₀-triazole-TrAIP-II) showed dose-dependent inhibition of Agr QS (FIG. 3b). The Alkyne-TrAIP-II was the most potent of the compounds (Table 6). It was presumed that modification of the inhibitor with the alkyne at the N-terminus endowed the inhibitor with enhanced accessibility to the receptor and/or tighter binding.

QS inhibition by surface-bound TrAIP-II was examined. Introduction of the S. aureus reporter strain to the Surface-PEG₁₀₀₀₀-azide in the presence of AIP-I in solution at its EC₅₀ (30 nM) resulted in activation of Agr QS (FIG. 3c and FIG. 12). However, if in the presence of 30 nM AIP-I, the strain was presented to the Surface-PEG₁₀₀₀₀-triazole-TrAIP-II, QS inhibition occurred (FIG. 3c,d), albeit with modest activation occurring at T=6 h (FIG. 12 (i)). As controls, the AIP-I concentration was increased to 1μM in solution and it was found that this was sufficient to outcompete the surface-bound TrAIP-II, causing strong activation of Agr QS over the 6 h period of the experiment (FIG. 3c and FIG. 12 (ii)). This result indicated that inhibition by surface-bound TrAIP-II was competitive, as it was in solution. Furthermore, Agr QS inhibition did not significantly occur when S. aureus cells were added to the Surface-PEG₁₀₀₀₀-triazole-Linear-TrAIP-II (FIG. 3c and FIG. 12 (iv)), demonstrating the requirement for specific structural elements in the antagonist.

### Example 18

### Wild-Type S. aureus Responds To Surface-Attached QS Molecules

It was investigated whether surface-attached pro- and anti-QS molecules could control S. aureus behaviors in the more natural and clinically-relevant context of S. aureus strains that are capable of producing AIP-I³⁷, unlike the reporter strain used above. For this analysis, the fluorescent QS reporter genes were introduced into wild-type S. aureus agr-I (RN6390b) and S. aureus MRSA agr-I. In the presence of the Surface-PEG₁₀₀₀₀-azide, S. aureus agr-I activated Agr QS in response to the accumulation of endogenously produced AIP-I (FIG. 4a,b (i)). The Surface-PEG₁₀₀₀₀-triazole-AIP-I caused an earlier and higher magnitude induction than did the Surface-PEG₁₀₀₀₀-azide, showing that S. aureus agr-I responded to both the surface-tethered AIP-I and to its endogenously produced AIP-I (FIG. 4a,b (ii)). The attached wild-type cells clearly responded to the surface attached AIP-I (FIG. 4b (ii)). This event elicited the positive autoinduction feedback loop causing the surface-adhered S. aureus cells to induce production and release of endogenous, soluble, AIP-I autoinducer. That autoinducer, in turn, activated the Agr QS response in unattached S. aureus cells residing in solution above the surface as shown by their red fluorescence emission (FIG. 13a (ii)). Thus, attached cells, if they have the capacity to make autoinducer, can rapidly propagate the signal to neighboring, non-surface-adhered cells. The Surface-PEG₁₀₀₀₀-triazole-TrAIP-II repressed the Agr QS response of the surface-adhered S. aureus cells over the 6 h period of the experiment (FIG. 4a,b (iii)). Furthermore, the surface-attached TrAIP-II also repressed the QS response of the neighboring, non-surface-adhered S. aureus cells (FIG. 13a (iii)). Presumbly, surface-attached TrAIP-II, by repressing QS in the surface-adhered S. aureus cells, decreased their endogenous production of AIP-I, slowing the autoinduction feedback loop which, in turn, delayed signal propagation beyond the surface. Consistent with this interpretation, exogenous provision of 1 μM AIP-I in solution relieved inhibition from the Surface-PEG₁₀₀₀₀-triazole-TrAIP-II (FIG. 4a,b (iv)), confirming, as above, that the Surface-PEG₁₀₀₀₀-triazole-TrAIP-II functions competitively. Analogous results were obtained using the clinical pathogen S. aureus MRSA (FIG. 13b), which highlights the generality of these results and of this approach for regulating QS by surface modification.

Next, it was explored how the modified surfaces influence S. aureus biofilm colonization dynamics. A constitutively expressed mKO fluorescent reporter was introduced into the wild-type S. aureus agr-I strain and the strain was grown on the Surface-PEG₁₀₀₀₀-azide, the Surface-PEG₁₀₀₀₀-triazole-AIP-I, and the Surface-PEG₁₀₀₀₀-triazole-TrAIP-II (FIGS. 4c and 4d). Consistent with the QS transcriptional reporter results reported herein and with the known role for Agr QS in triggering biofilm dispersal in S. aureus, when grown on the Surface-PEG₁₀₀₀₀-triazole-AIP-I, the S. aureus agr-I strain induced biofilm dispersal, which resulted in an ˜80% reduction in biofilm coverage compared to when the strain was grown on the Surface-PEG₁₀₀₀₀-azide. When the S. aureus agr-I strain was grown on the Surface-PEG₁₀₀₀₀-triazole-TrAIP-II, repression of Agr quroum sensing increased biofilm coverage ˜2-fold compared to when the strain was grown on the Surface-PEG₁₀₀₀₀-azide. Analogous results were obtained using the wild-type S. aureus agr-II (i.e, subgroup II: RN6607) strain grown on the Surface-PEG₁₀₀₀₀-azide, the Surface-PEG₁₀₀₀₀-triazole-AIP-II, and the Surface-PEG₁₀₀₀₀-triazole-TrAIP-II (FIG. 13c). Specifically, in the S. aureus agr-II strain, Agr QS was activated by the surface-attached AIP-II, which resulted in biofilm dispersal, and QS was inhibited by the Surface-PEG₁₀₀₀₀-triazole-TrAIP-II, which resulted in increased biofilm coverage of the surface.

### Example 19

### Applications of Surface-Attached QS Molecules

To expand these results to other surfaces, gold and polydimethylsiloxane (PDMS) surfaces were chemically modified with the dye-triazole-PEG moieties (FIG. 13d). Given the effectiveness and specificity of the click reaction, preliminary results with the dye molecules suggested that AIP-I or TrAIP-II could be attached to these two surfaces. These results suggested that the logic here is general and can be broadened to other materials as well as other molecules of interest.

Having a reasonable shelf lifetime was also desirable for chemically coated surfaces in medical and industrial applications. The long-term stability of the Surface-PEG₁₀₀₀₀-triazole-dye and the Surface-PEG₁₀₀₀₀-triazole-AIP-I as a proxy for modified surface longevity was examined. Irrespective of the presence or absence of S. aureus cells, the intensity of the surface-attached dye did not change for 15 h (FIG. 5a). In storage, in the absence of cells, the intensity did not change for 40 days (FIG. 13e (i)), and furthermore, the response of S. aureus cells to AIP-I-attached surfaces that had been stored for 40 days was identical to that of freshly generated surfaces (FIG. 13e (ii)). These findings indicated that the surface-tethered entities do not detach over time and the components remain stable for long time periods. These results are especially important in the context of materials such as submerged prosthetics, as these results suggested that concerns about cytotoxicity due to leaching or instability may not be highly relevant to this strategy.

To further explore issues pertaining to medical applications of modified surfaces, QS analysis in the presence of human blood plasma was performed because S. aureus frequently causes bacteremia. Having blood plasma present throughout the experiment did not alter the ability of the AIP-I-coated surface to stimulate S. aureus QS (FIG. 13f). The long-term effectiveness of surface-attached QS molecules to repeated “infection” was investigated. The S. aureus reporter strain was introduced to the Surface-PEG₁₀₀₀₀-triazole-AIP-I (FIG. 5b (i)) and after 3 h, the S. aureus cells were removed by repeatedly introducing flow (σₛₕₑₐᵣ=0.03-0.3 Pa) and air-bubbles³⁸. A second dose of S. aureus reporter cells were introduced to the surface (FIG. 5b (i,ii)). The two S. aureus reporter strains were labeled with different constitutive fluorescent colors so each could be monitored. Cells from both the first and second inoculations colonized the same region (FIG. 5b (iii)), and both responded to the modified surface, activating Agr QS to a comparable magnitude (FIG. 5b (iv)). Thus, the surface-attached molecules remained effective following mechanical shear. This feature was especially important in the biomedical arena in which long-term activity of surface-attached molecules is essential to combat repeated infection. Finally, the potential to generate multi-functional surfaces by simultaneously attaching an equal mixture of red and green dyes to the same surface (FIG. 5c) was demonstrated. As noted, there are an estimated 2.1×10⁴ μm⁻² sites on the surface available for attachment to biomolecules (FIG. 11e). However, an AIP-I surface coverage density of 1.6×10⁴ μm⁻² was sufficient to fully induce S. aureus Agr QS (FIG. 11e). Thus, approximately 25% of the sites (or 0.5×10⁴ μm⁻²) were not required to achieve the maximal QS response. These sites could conceivably be used to attach a different biomolecule, for example, one with an orthogonal activity. Thus, the preliminary results suggested surprising versatility in this approach: QS molecules could be simultaneously or sequentially attached in combination with other biomolecules, such as antimicrobial agents, enzymes, or perhaps QS compounds that target other bacteria³⁹⁻⁴¹.

### Example 20

### Solid Phase Peptide Synthesis and Peptide Characterization

2-chloro-trityl chloride resin was first derivatized with hydrazine monohydrate in dimethylformamide (DMF) for activation. Amino acids were coupled through standard Fmoc-based solid phase peptide synthesis. Note that the cysteine was protected with—StBu (or tert-butyl-thiolate). 4-pentynoic acid (5 equiv.) was activated by N,N′-diisopropyl carbodiimide (DIC, 20 equiv.) in 10 ml of CH₂Cl₂ under N2 for 30 min before coupling to the peptide on resin for another 2 h^{S10}. The peptide was cleaved from the resin by stirring with a cleavage cocktail (95% TFA, 2.5% TIPS, and 2.5% H₂O) at room temperature for 2 h. The post-cleavage supernatant was precipitated with cold ether. The resulting pellet was washed twice with cold ether, dried under N2 and purified by preparative reverse-phased high performance liquid chromatography (RP-HPLC) on a C18 column.

The cleaved and purified hydrazide peptide was subjected to oxidation and thioester formation at the C-terminus^{S11}. In the oxidation step, the peptide was dissolved in degassed oxidation buffer (6M guanidine hydrochloride and 100 mM phosphate at pH=3) to a final concentration of 5 mM before reaction with 20 equiv. NaNO₂ for 20-30 min in a −10° C. ice-salt water bath. Next, the system was brought back to room temperature and mixed with 100 mM MESNa dissolved in thiol buffer (6M guanidine hydrochloride and 100 mM phosphate) for in situ thioesterification. The pH was adjusted to 6. After running for 2 h, the reaction products were purified by semi-preparative RP-HPLC. The thioester precursor was lyophilized and redissolved in cyclization buffer (50 mM TCEP, 100 mM phosphate of pH 7, 25% ACN) for cysteine deprotection and thiolactone formation^{S12}. The reaction was allowed to run for 2 h at room temperature, followed by purification through semi-preparative RP-HPLC and characterization through analytical RP-HPLC and ESI-MS (FIG. 8).

For generation of PEG₃₃₀-triazole-AIP-I and PEG₃₃₀-triazole-TrAIP-II, the CuAAC click reaction was performed to incorporate the O-(2-azidoethyl)heptaethylene glycol onto the Alkyne-AIP derivatives. The alkyne-AIPs and PEG₃₃₀-azide were brought to solution in 100 mM phosphate buffer at pH 7 at 200 μM and 400 μM, respectively. Copper catalyst and ligands were added at final concentrations of 0.10 mM CuSO₄ and 0.50 mM Tris(3-hydroxypropyltriazolylmethyl)amine, and subsequently treated with 5 mM sodium ascorbate. The reaction was mixed well and carried out for 1 h. At the end of the reaction, the system was quenched with 2 mM EDTA before purification by semi-prep RP-HPLC and characterization through analytical RP-HPLC and ESI-MS.

### Example 21

### Quantification of Peptide Concentration

All Alkyne-AIPs and PEG₃₃₀-triazole-AIPs were quantified by ¹H-NMR^{S13}. This was a simple approach to determine sample concentration using a standard capillary and an external calibration sample of known concentration. First, 150 μM of DSS was dissolved in D20 as the reference material for the standard capillary. Next, 3 mM glycine dissolved in D₂O was used as the calibration sample. Finally, the peptide samples in deuterated DMSO (DMSO-d6) were prepared.

All ¹H NMR were performed on a 500 MHz NMR. The NMR spectrum of the glycine calibration sample containing the DSS standard capillary was acquired first, followed by that of the peptide sample containing the same capillary. The intensities of the peaks of interest and of the reference capillary in the samples were determined through MNova (MestreLab) data processing software. In this study, peaks in the corresponding to aromatic protons (from Tyr in AIP-I and Phe in TrAIP-II) were integrated.

The concentration of any sample of interest was determined using equation PI I is the integral (or sum of integrals) of the peak(s) of interest, #P is the number of protons that contribute to the peaks of interest per molecule, and C is the overall concentration of the material in solution. The calibration material is glycine, where the peak of interest corresponds to the two protons at the alpha position. The capillary material is DSS with the peak of interest arising from the nine silicon-shielded methyl protons.

$${C}_{sample} = {C}_{calibration material} \times \frac{\# {P}_{calibration material}}{\# {P}_{sample}} = \frac{\frac{{I}_{sample}}{{I}_{capillary in sample}}}{\frac{{I}_{calibration material}}{{I}_{capillary in calibration material}}} \qquad [ 1 ]$$

The derivation of equation [1] rested on the assumption that each individual proton in the sample contributes the same amount of area to the peak integral, and that #P_{capillary}×C_{capillary} is constant for both the calibration sample and the sample of interest because the same capillary insert is used for the two samples.

### REFERENCES

1 Wang, B. & Muir, T. W. Regulation of virulence in Staphylococcus aureus: molecular mechanisms and remaining puzzles. Cell Chem Biol 23, 214-224 (2016).
2 Kong, K. F., Vuong, C. & Otto, M. Staphylococcus quorum sensing in biofilm formation and infection. Int J Med Microbiol 296, 133-139 (2006).
3 Drescher, K. et al. Architectural transitions in Vibrio cholerae biofilms at single-cell resolution. Proc Natl Acad Sci USA 113, 2066-2072 (2016).
4 Yan, J., Sharo, A. G., Stone, H. A., Wingreen, N. S. & Bassler, B. L. Vibrio cholerae biofilm growth program and architecture revealed by single-cell live imaging. Proc Natl Acad Sci USA 113, 5337-5343 (2016).
5 Nadell, C. D., Drescher, K. & Foster, K. R. Spatial structure, cooperation and competition in biofilms. Nat Rev Microbiol 14, 589-600 (2016).
6 Waters, C. M. & Bassler, B. L. Quorum sensing: cell-to-cell communication in bacteria. Annu Rev Cell Dev Biol 21, 319-346 (2005).
7 Papenfort, K. & Bassler, B. L. Quorum sensing signal-response systems in Gram-negative bacteria. Nat Rev Microbiol 14, 576-588 (2016).
8 Novick, R. P. & Geisinger, E. Quorum sensing in Staphylococci. Annu Rev Genet 42, 541-564 (2008).
9 Dinges, M. M., Orwin, P. M. & Schlievert, P. M. Exotoxins of Staphylococcus aureus. Clin Microbiol Rev 13, 16-34 (2000).
10 Arvidson, S. & Tegmark, K. Regulation of virulence determinants in Staphylococcus aureus. Int J Med Microbiol 291, 159-170 (2001).
11 Hammer, B. K. & Bassler, B. L. Quorum sensing controls biofilm formation in Vibrio cholerae. Mol Microbiol 50, 101-104 (2003).
12 Hsiao, A. et al. Members of the human gut microbiota involved in recovery from Vibrio cholerae infection. Nature 515, 423-426 (2014).
13 Ismail, A. S., Valastyan, J. S. & Bassler, B. L. A host-produced autoinducer-2 mimic activates bacterial quorum sensing. Cell Host Microbe 19, 470-480 (2016).
14 O′Loughlin, C. T. et al. A quorum-sensing inhibitor blocks Pseudomonas aeruginosa virulence and biofilm formation. Proc Natl Acad Sci USA 110, 17981-17986 (2013).
15 Sully, E. K. et al. Selective chemical inhibition of agr quorum sensing in Staphylococcus aureus promotes host defense with minimal impact on resistance. Plos Pathog 10 (2014).
16. Otto, M. Quorum-sensing control in Staphylococci—a target for antimicrobial drug therapy? Fems Microbiol Lett 241, 135-141 (2004).
17 Cegelski, L., Marshall, G. R., Eldridge, G. R. & Hultgren, S. J. The biology and future prospects of antivirulence therapies. Nature Reviews Microbiology 6, 17-27 (2008).
18 Kim, M. K., Ingremeau, F. B., Zhao, A., Bassler, B. L. & Stone, H. A. Local and global consequence of flow on bacterial quorum sensing. Nature Microbiology 1, 15005 (2016).
19 Lu, H. D. et al. Modulating Vibrio cholerae quorum-sensing-controlled communication using autoinducer-loaded nanoparticles. Nano Lett 15, 2235-2241 (2015).
20 Persat, A. et al. The mechanical world of bacteria. Cell 161, 988-997 (2015).
21 Kim, M. K., Drescher, K., Pak, O. S., Bassler, B. L. & Stone, H. A. Filaments in curved streamlines:rapid formation of Staphylococcus aureus biofilm streamers. New J Phys 16 (2014).
22 Broderick, A. H. et al. Surface coatings that promote rapid release of peptide-based AgrC inhibitors for attenuation of quorum sensing in Staphylococcus aureus. Adv Healthc Mater 3, 97-105 (2014).
23 Ho, K. K. K. et al. Quorum sensing inhibitory activities of surface immobilized antibacterial dihydropyrrolones via click chemistry. Biomaterials 35, 2336-2345 (2014).
24 Lowy, F. D. Staphylococcus aureus infections. New Engl J Med 339, 2026-2027 (1998).
25 Gordon, R. J. & Lowy, F. D. Pathogenesis of methicillin-resistant Staphylococcus aureus infection. Clin Infect Dis 46, S350-S359 (2008).
26 Zhang, L. S., Gray, L., Novick, R. P. & Ji, G. Y. Transmembrane topology of AgrB, the protein involved in the post-translational modification of AgrD in Staphylococcus aureus. J Biol Chem 277, 34736-34742 (2002).
27 Wang, B. Y., Zhao, A. S., Novick, R. P. & Muir, T. W. Key driving forces in the biosynthesis of autoinducing peptides required for staphylococcal virulence. P Natl Acad Sci USA 112, 10679-10684 (2015).
28 Lina, G. et al. Transmembrane topology and histidine protein kinase activity of AgrC, the agr signal receptor in Staphylococcus aureus. Mol Microbiol 28, 655-662 (1998).
29 Wang, B. Y., Zhao, A. S., Novick, R. P. & Muir, T. W. Activation and inhibition of the receptor histidine kinase AgrC occurs through opposite helical transduction motions. Mol Cell 53, 929-940 (2014).
30 Koenig, R. L., Ray, J. L., Maleki, S. J., Smeltzer, M. S. & Hurlburt, B. K. Staphylococcus aureus AgrA binding to the RNAIII-agr regulatory region. J Bacteriol 186, 7549-7555 (2004).
31. Fechter, P., Caldelari, I., Lioliou, E. & Romby, P. Novel aspects of RNA regulation in Staphylococcus aureus. Febs Lett 588, 2523-2529 (2014).
32 Lyon, G. J., Wright, J. S., Muir, T. W. & Novick, R. P. Key determinants of receptor activation in the agr autoinducing peptides of Staphylococcus aureus. Biochemistry-Us 41, 10095-10104 (2002).
33 Hong, V., Presolski, S. I., Ma, C. & Finn, M. G. Analysis and optimization of copper-catalyzed azide-alkyne cycloaddition for bioconjugation. Angew Chem Int Edit 48, 9879-9883 (2009).
34 Emilsson, G. et al. Strongly stretched protein resistant poly(ethylene glycol) brushes prepared by grafting-to. Acs Appl Mater Inter 7, 7505-7515 (2015).
35 Cisar, E. A., Geisinger, E., Muir, T. W. & Novick, R. P. Symmetric signalling within asymmetric dimers of the Staphylococcus aureus receptor histidine kinase AgrC. Mol Microbiol 74, 44-57 (2009).
36 Vollmer, W., Blanot, D. & de Pedro, M. A. Peptidoglycan structure and architecture. Fems Microbiol Rev 32, 149-167 (2008).
37 Traber, K. E. et al. agr function in clinical Staphylococcus aureus isolates. Microbiol-Sgm 154, 2265-2274 (2008).
38 Khodaparast, S., Kim, M. K., Silpe, J. & Stone, H. A. Bubble-driven detachment of bacteria from confined micro-geometries. Submitted (2016).
39 Vasilev, K., Cook, J. & Griesser, H. J. Antibacterial surfaces for biomedical devices. Expert Rev Med Devic 6, 553-567 (2009).
40 Gallo, J., Holinka, M. & Moucha, C. S. Antibacterial surface treatment for orthopaedic implants. Int J Mol Sci 15, 13849-13880 (2014).
41 Lee, J. J. et al. Synthetic ligand-coated magnetic nanoparticles for microfluidic bacterial separation from blood. Nano Lett 14, 1-5 (2014).
42 Boles, B. R. & Horswill, A. R. agr-mediated dispersal of Staphylococcus aureus biofilms. Plos Pathog 4 (2008).
43 Davies, D. Understanding biofilm resistance to antibacterial agents. Nat Rev Drug Discov 2, 114-122 (2003).
44 Huycke, M. M., Spiegel, C. A. & Gilmore, M. S. Bacteremia caused by hemolytic, high-level gentamicin-resistant Enterococcus faecalis. Antimicrob Agents Ch 35, 1626-1634 (1991).
45 Cook, L. C. & Federle, M. J. Peptide pheromone signaling in Streptococcus and Enterococcus. Ferns Microbiol Rev 38, 473-492 (2014).
46 Mylonakis, E. et al. The Enterococcus faecalis fsrB gene, a key component of the fsr quorum-sensing system, is associated with virulence in the rabbit endophthalmitis model. Infect Immun 70, 4678-4681 (2002).
47 Nakayama, J. et al. Development of a peptide antagonist against fsr quorum sensing of Enterococcus faecalis. ACS Chem Biol 8, 804-811 (2013).
48 Gray, B., Hall, P. & Gresham, H. Targeting agr- and agr-like quorum sensing systems for development of common therapeutics to treat multiple Gram-positive bacterial infections. Sensors-Basel 13, 5130-5166 (2013).
49 Fujii, T. et al. Two homologous agr-like quorum-sensing systems cooperatively control adherence, cell morphology, and cell viability properties in Lactobacillus plantarum WCFS1. J Bacteriol 190, 7655-7665 (2008).
50 Wang, I. N., Smith, D. L. & Young, R. Holins: The protein clocks of bacteriophage infections. Annu Rev Microbiol 54, 799-825 (2000).
51 Goedhart, J. et al. Structure-guided evolution of cyan fluorescent proteins towards a quantum yield of 93%. Nature Communications 3 (2012).
52 Nadell, C. D., Drescher, K., Wingreen, N. S. & Bassler, B. L. Extracellular matrix structure governs invasion resistance in bacterial biofilms. Isme Journal 9, 1700-1709 (2015).
53 Chen, J., Yoong, P., Ram, G., Tones, V. J. & Novick, R. P. Single-copy vectors for integration at the SaPIl attachment site for Staphylococcus aureus. Plasmid 76, 1-7 (2014).
54 Lyon, G. J., Mayville, P., Muir, T. W. & Novick, R. P. Rational design of a global inhibitor of the virulence response in Staphylococcus aureus, based in part on localization of the site of inhibition to the receptor-histidine kinase, AgrC. P Natl Acad Sci USA 97, 13330-13335 (2000).
S1 Kreiswirth, B. N. et al. The toxic shock syndrome exotoxin structural gene is not detectably transmitted by a prophage. Nature 305, 709-712 (1983).
S2 Ruzin, A., Lindsay, J. & Novick, R. P. Molecular genetics of SaP11—a mobile pathogenicity island in Staphylococcus aureus. Mol Microbiol 41, 365-377 (2001).
S3 Novick, R. P. Genetic Systems in Staphylococci. Method Enzymol 204, 587-636 (1991).
S4 Novick, R. P. et al. Synthesis of staphylococcal virulence factors is controlled by a regulatory RNA molecule. Embo J12, 3967-3975 (1993).
S5 Kim, M. K., Ingremeau, F. B., Zhao, A., Bassler, B. L. & Stone, H. A. Local and global consequences of flow on bacterial quorum sensing. Nature Microbiology 1, 15005 (2016).
S6 Lyon, G. J., Mayville, P., Muir, T. W. & Novick, R. P. Rational design of a global inhibitor of the virulence response in Staphylococcus aureus, based in part on localization of the site of inhibition to the receptor-histidine kinase, AgrC. P Natl Acad Sci USA 97, 13330-13335 (2000).
S7 Geisinger, E., George, E. A., Muir, T. W. & Novick, R. P. Identification of ligand specificity determinants in AgrC, the Staphylococcus aureus quorum-sensing receptor. J Biol Chem 283, 8930-8938 (2008).
S8 Charpentier, E. et al. Novel cassette-based shuttle vector system for gram-positive bacteria. Appl Environ Microb 70, 6076-6085 (2004).
S9 Novick, R. P. & Geisinger, E. Quorum sensing in Staphylococci. Annu Rev Genet 42, 541-564 (2008).
S10 Lu, J., Shi, M. & Shoichet, M. S. Click chemistry functionalized polymeric nanoparticles target corneal epithelial cells through RGD-cell surface receptors. Bioconjugate Chem 20, 87-94 (2009).
S11 Zheng, J. S., Tang, S., Qi, Y. K., Wang, Z. P. & Liu, L. Chemical synthesis of proteins using peptide hydrazides as thioester surrogates. Nat Protoc 8, 2483-2495 (2013).
S12 George, E. A., Novick, R. P. & Muir, T. W. Cyclic peptide inhibitors of staphylococcal virulence prepared by Fmoc-based thiolactone peptide synthesis. J Am Chem Soc 130, 4914-4924 (2008).
S13 Fardink, J. R. Dr. Bence: A simple method for determining absolute concentration by ¹H NMR. Department of Chemistry, Princeton University (2010). Link: http://arks.princeton.edu/ark:/88435/dsp01m039k5538.

What is claimed is:

1. A surface comprising a quorum sensing (“QS”) modulating molecule attached to the surface by a linker.

2. The surface of claim 1, wherein:
a. the length of the linker is sufficient to traverse a bacterial cell's peptidoglycan layer, outer membrane layer or both;
b. the QS modulating molecule binds to a receptor on a cell membrane of a bacterial cell;
c. the linker has a diameter of less than 5 nm;
d. the linker has a length greater than 1, 2, 3, 4, 5, 6, 7, 8, 9, 10, 11, 12, 13, 14 or 15 nm;
e. the linker is a chemical bound;
f. the linker is selected from polyethylene glycol (PEGs), polyphosphazenes, polylactide, polyglycolide, polycaprolactone, or any other combinations thereof;
g. the linker is attached to the surface using one or more of the following types of chemical reactions: silanization, gold-sulfide bond formation, thiol-ene reactions, and surface-initiated polymerization;
h. the average surface coverage density of the QS modulating molecule is of a sufficient density to modulate QS;
i. the average surface coverage density of the QS modulating molecule is about 2.1×10² μm⁻² or greater;
j. the QS modulating molecule comprises an antagonist of QS that alters QS-controlled phenotypes of biofilm production, biofilm streamer production, and/or virulence factor production;
k. the QS modulating molecule comprises an agonist of QS that alters QS-controlled phenotypes of biofilm production, biofilm streamer production and/or virulence factor production;
l. the QS modulating molecule retains modulating activity after being stored at about 4° C. for up to 40 days;
m. the QS modulating molecule remains bound to the surface via the linker after exposure to laminar fluid flow;
n. the QS modulating molecule exhibits modulating activity when exposed to a population of bacterial cells;
o. the QS modulating molecule exhibits modulating activity when reexposed to another population of bacterial cells;
p. the QS modulating molecule is a molecule selected from Tables 1A or 1B or its derivative molecules;
q. the QS modulating molecule is attached to the linker using one or more of the following types of chemical reactions: biorthogonal reactions, click chemistry, thiol-ene reactions, gold-sulfide bond formation, esterification reactions, Grignard reactions, Michael reactions, ketone/hydroxylamine condensations, Staudinger ligations, strain-promoted alkyne-azide cycloadditions, photo-click cycloadditions, Diels-Alder cycloadditions, tetrazine-alkene/alkyne cycloadditions, Cu-catalyzed alkyne-azide cycloadditions, Pd-catalyzed cross coupling, strain promoted alkyne-nitrone cycloadditions, Cross-metathesis, Norbornene cycloadditions, Oxanorbornadiene cycloadditions, tetrazine ligations, or tetrazole photoclick chemistry;
r. the surface comprises glass, metal, stainless metal, silicon, plastic, polymer, metal, or ceramic material or any combination thereof;
s. the surface is a small particle, a nanoparticle, a flat surface or a curved surface; or
t. any combination of (a)-(s).

3. The surface of claim 2, wherein the bacterial cell is Gram-negative, Gram-positive or a mixture of Gram-negative and Gram-positve.

4. The surface of claim 2, wherein the bacterial cell is exposed to a permeability agent that forms holes in the outer membrane layer of the bacterial cell prior to contacting the surface.

5. The surface of claim 2, wherein:
a. the polymer is selected from polyethylene, polypropylene, polystyrene, polyester, polyester PLA and other biosorbable plastics, polycarbonate, polyvinyl chloride, polyethersulfone, polyacrylate (e.g., Acrylic, PMMA), hydrogel (e.g., acrylate), polysulfone, polyetheretherketone, thermoplastic elastomers (e.g., TPE, TPU), thermoset elastomers, silicone, poly-p-xylylene (e.g., Parylene), fluoropolymers;
b. the metal is selected from stainless steel, cobalt-base alloy, titanium, titanium-base alloy, and/or shape memory alloy; and/or
c. the ceramic material comprises glass ceramic, calcium phosphate ceramic, and/or carbon-based ceramic.

6. The surface of claim 2, wherein the surface comprises a second QS modulating molecule attached the surface by a second linker.

7. The surface of claim 6, wherein:
a. the length of the second linker is sufficient to traverse the bacterial cell's peptidoglycan layer, outer membrane layer or both;
b. the second QS modulating molecule competitively binds to the receptor on the cell membrane of the bacterial cell;
c. wherein the second QS modulating molecule binds to a different receptor on the cell membrane;
d. the second linker has a diameter of less than 5 nm;
e. the second linker has a length greater than 15 nm;
f. the second linker is a chemical bound;
g. the second linker is selected from polyethylene glycol (PEGs), polyphosphazenes, polylactide, polyglycolide, polycaprolactone, or any other combinations thereof;
h. the second linker is attached to the surface using one or more of the following types of chemical reactions: silanization, gold-sulfide bond formation, thiol-ene reactions, and surface-initiated polymerization;
i. the second linker is the same as the linker;
j. the second linker is different from the linker;
k. any combination of (a)-(j).

8. The surface of claim 1, wherein the surface is placed in an environment.

9. The surface of claim 8, wherein the environment is:
a. static;
b. under pressure;
c. a flow environment;
d. under controlled pressure; and/or e. an implantable medical device, part of machinery used in industrial processes, a culvert, a pool used in a waste water treatment facility, waste water treatment facility, a pipe, a cooling tower, a medical device, industrial fluid handling machinery, a wound, within the body, a medical process, an agricultural process, and/or machinery.

10. A method of modulating QS, biofilm formation, biofilm streamer formation, and/or a virulence factor production by a microorganism, wherein the method comprises contacting a microorganism with the surface of claim 1.

11. The method of claim 10, wherein the method inhibits a pathogenic behavior of a microorganism.

12. The method of claim 10, wherein the method promotes a beneficial behavior of a microorganism.

13. The method of claim 10, wherein the microorganism is selected from bacteria, archaea, protozoa, fungi, and/or algae.

14. The method of claim 13, wherein the bacteria is selected from Abiotrophia, Achromobacter, Acidaminococcus, Acidovorax, Acinetobacter, Actinobacillus, Actinobaculum, Actinomadura, Actinomyces, Aerococcus, Aeromonas, Afipia, Agrobacterium, Alcaligenes, Alloiococcus, Alteromonas, Amycolata, Amycolatopsis, Anabaena, Anabaenopsis, Anaerobospirillum, Anaerorhabdus, Aphanizomenon, Arachnia, Arcanobacterium, Arcobacter, Arthrobacter, Atopobium, Aureobacterium, Bacillus, Bacteroides, Balneatrix, Bartonella, Bergeyella, Bifidobacterium, Bilophila, Bordetella, Borrelia, Brachyspira, Branhamella, Brevibacillus, Brevibacterium, Brevundimonas, Brucella, Burkholderia, Buttiauxella, Butyrivibrio, Calymmatobacterium, Camesiphon, Campylobacter, Capnocytophaga, Capnylophaga, Cardiobacterium, Catonella, Cedecea, Cellulomonas, Centipeda, Chlamydia, Chlamydophila, Chromobacterium, Chryseomonas, Chyseobacterium, Citrobacter, Clostridium, Collinsella, Comamonas, Corynebacterium, Coxiella, Cryptobacterium, Cyanobacteria, Cylindrospermopsis, Delftia, Dermabacter, Dermatophilus, Desulfomonas, Desulfovibrio, Dialister, Dichelobacter, Dolosicoccus, Dolosigranulum, Edwardsiella, Eggerthella, Ehrlichia, Eikenella, Empedobacter, Enterobacter, Enterococcus, Erwinia, Erysipelothrix, Escherichia, Eubacterium, Ewingella, Exiguobacterium, Facklamia, Filifactor, Flavimonas, Flavobacterium, Francisella, Fusobacterium, Gardnerella, Gemella, Globicatella, Gloeobacter, Gordona, Haemophilus, Hafnia, Hapalosiphon, Helicobacter, Helococcus, Hemophilus, Holdemania, Ignavigranum, Johnsonella, Kingella, Klebsiella, Kocuria, Koserella, Kurthia, Kytococcus, Lactobacillus, Lactococcus, Lautropia, Leclercia, Legionella, Leminorella, Leptospira, Leptospirae, Leptotrichia, Leuconostoc, Listeria, Listonella, Lyngbya, Megasphaera, Methylobacterium, Microbacterium, Micrococcus, Microcystis, Mitsuokella, Mobiluncus, Moellerella, Moraxella, Morganella, Mycobacterium, Mycoplasma, Myroides, Neisseria, Nocardia, Nocardiopsis, Nodularia, Nostoc, Ochrobactrum, Oeskovia, Oligella, Orientia, Paenibacillus, Pantoea, Parachlamydia, Pasteurella, Pediococcus, Peptococcus, Peptostreptococcus, Phormidium, Photobacterium, Photorhabdus, Phyllobacterium, Phytoplasma, Planktothrix, Plesiomonas, Porphyromonas, Prevotella, Propionibacterium, Proteus, Providencia, Pseudoanabaena, Pseudomonas, Pseudonocardia, Pseudoramibacter, Psychrobacter, Rahnella, Ralstonia, Rhodococcus, Rickettsia, Rochalimaea, Roseomonas, Rothia, Ruminococcus, Salmonella, Schizothrix, Selenomonas, Serpulina, Serratia, Shewenella, Shigella, Simkania, Slackia, Sphaerotilus, Sphingobacterium, Sphingomonas, Spirillum, Spiroplasma, Spirulina, Staphylococcus, Stenotrophomonas, Stomatococcus, Streptobacillus, Streptococcus, Streptomyces, Succinivibrio, Sutterella, Suttonella, Tatumella, Tissierella, Trabulsiella, Treponema, Trichodesmium, Tropheryma, Tsakamurella, Turicella, Umezakia, Ureaplasma, Vagococcus, Veillonella, Vibrio, Weeksella, Wolinella, Xanthomonas, Xenorhabdus, Yersinia, Yokenella. Acinetobacter baumannii, Actinobacillus actinomycetemcomitans, Actinobacillus pleuropneumoniae, Actinomyces bovis, Actinomyces israelii, Bacillus anthracis, Bacillus ceretus, Bacillus coagulans, Bacillus liquefaciens, Bacillus popillae, Bacillus subtilis, Bacillus thuringiensis, Bacteroides distasonis, Bacteroides fragilis, Bacteroides thetaiotaomicron, Bacteroides vulgatus, Bartonella bacilliformis, Bartonella Quintana, Beneckea parahaemolytica, Bordetella bronchiseptica, Bordetella parapertussis, Bordetella pertussis, Borelia burgdorferi, Brevibacterium lactofermentum, Brucella abortus, Brucella canis, Brucella melitensis, Brucella suis, Burkholderia cepacia, Burkholderia mallei, Burkholderia pseudomallei, Campylobacter fetus, Campylobacter jejuni, Campylobacter pylori, Cardiobacterium hominis, Chlamydia pneumoniae, Chlamydia psittaci, Chlamydia trachomatis, Chlamydophila abortus, Chlamydophila caviae, Chlamydophila felis, Chlamydophila pneumonia, Chlamydophila psittaci, Chryseobacterium eningosepticum, Clostridium botulinum, Clostridium butyricum, Clostridium coccoides, Clostridium difficile, Clostridium leptum, Clostridium tetani, Corynebacterium xerosis, Cowdria ruminantium, Coxiella burnetii, Edwardsiella tarda, Ehrlichia sennetsu, Eikenella corrodens, Elizabethkingia meningoseptica, Enterobacter aerogenes, Enterobacter cloacae, Enterococcus faecalis, Escherichia coli, Escherichia hirae, Flavobacterium meningosepticum, Fluoribacter bozemanae, Francisella tularensis, Francisella tularensis biovar Tularensis, Francisella tularensis subsp. Holarctica, Francisella tularensis subsp. nearctica, Francisella tularensis subsp. Tularensis, Francisella tularensis var. palaearctica, Fudobascterium nucleatum, Fusobacterium necrophorum, Haemophilus ducreyi, Haemophilus influenzae, Helicobacter pylori, Kingella kingae, Klebsiella mobilis, Klebsiella oxytoca, Klebsiella pneumoniae, Lactobacillus acidophilus, Lactobacillus casei, Lactobacillus hilgardii, Lactobacillus pentosus, Lactobacillus plantarum, Lactobacillus rhamnosus, Lactococcus lactis, Legionella bozemanae corrig., Legionella pneumophila, Leptospira alexanderi, Leptospira borgpetersenii, Leptospira fainei, Leptospira inadai, Leptospira interrogans, Leptospira kirschneri, Leptospira noguchii, Leptospira santarosai, Leptospira weilii, Leuconostoc lactis, Leuconostoc oenos, Listeria ivanovii, Listeria monocytogenes, Moraxella catarrhalis, Morganella morganii, Mycobacterium africanum, Mycobacterium avium, Mycobacterium avium subspecies paratuberculosis, Mycobacterium bovis, Mycobacterium bovis strain BCG, Mycobacterium intracellulare, Mycobacterium kansasii, Mycobacterium leprae, Mycobacterium marinum, Mycobacterium tuberculosis, Mycobacterium typhimurium, Mycobacterium ulcerans, Mycoplasma hominis, Mycoplasma mycoides, Mycoplasma pneumoniae, Neisseria gonorrhoeae, Neisseria meningitidis, Neorickettsia sennetsu, Nocardia asteroides, Orientia tsutsugamushi, Pasteurella haemolytica, Pasteurella multocida, Plesiomonas shigelloides, Propionibacterium acnes, Proteus mirabilis, Proteus morganii, Proteus penneri, Proteus rettgeri, Proteus vulgaris, Providencia alcalifaciens, Providencia rettgeri, Pseudomonas aeruginosa, Pseudomonas mallei, Pseudomonas pseudomallei, Pyrococcus abyssi, Rickettsia akari, Rickettsia canadensis, Rickettsia canadensis corrig, Rickettsia conorii, Rickettsia montanensis, Rickettsia montanensis corrig, Rickettsia prowazekii, Rickettsia rickettsii, Rickettsia sennetsu, Rickettsia tsutsugamushi, Rickettsia typhi, Rochalimaea quintana, Salmonella arizonae, Salmonella choleraesuis subsp. arizonae, Salmonella enterica subsp. Arizonae, Salmonella enteritidis, Salmonella paratyphi, Salmonella typhi, Salmonella typhimurium, Selenomonas nominantium, Selenomonas ruminatium, Serratia marcescens, Shigella boydii, Shigella dysenteriae, Shigella flexneri, Shigella sonnei, Spirillum minus, Staphylococcus aureus, Staphylococcus epidermidis, Staphylococcus equi, Staphylococcus lugdunensis, Stenotrophomonas maltophila, Streptobacillus moniliformis, Streptococcus agalactiae, Streptococcus bovis, Streptococcus ferus, Streptococcus pneumoniae, Streptococcus pyogenes, Streptococcus viridans, Streptomyces ghanaenis, Streptomyces hygroscopicus, Streptomyces phaechromogenes, Treponema carateum, Treponema denticola, Treponema pallidum, Treponema pertenue, Vibrio cholerae, Vibrio parahaemolyticus, Vibrio vulnificus, Xanthomonas maltophilia, Yersinia enterocolitica, Yersinia pestis, Yersinia pseudotuberculosis, Zymomonas mobilis, or Fusospirochetes.

15. The method of claim 13, wherein the fungi is selected from Candida, Saccharomyces, or Cryptococcus.

16. The method of claim 10, wherein the method is used to treat sepsis, pneumonia, infections from cystic fibrosis, otitis media, chronic obstructive pulmonary disease, a urinary tract infection, periodontal disease, gingivitis, periodontitis, breath malodor, treat infections, Gram-negative infections, Gram-positive infections, otitis media, prostatitis, cystitis, bronchiectasis, bacterial endocarditis, osteomyelitis, dental caries, periodontal disease, infectious kidney stones, acne, Legionnaire's disease, chronic obstructive pulmonary disease (COPD), cystic fibrosis, an accumulation of biofilm in the lungs or digestive tract, emphysema, chronic bronchitis, also encompasses infections on implanted/inserted devices, medical device-related infections, biliary stent infections, orthopedic implant infections, catheter-related infections, skin infections, dermatitis, ulcers from peripheral vascular disease, a burn injury, trauma, rosacea, skin infection, pneumonia, otitis media, sinusitus, bronchitis, tonsillitis, and mastoiditis related to infection by Streptococcus pneumoniae, Haemophilus influenzae, Moraxella catarrhalis, Staphylococcus aureus, Peptostreptococcus spp. or Pseudomonas spp.; pharynigitis, rheumatic fever, and glomerulonephritis related to infection by Streptococcus pyogenes, Groups C and G streptococci, Clostridium diptheriae, or Actinobacillus haemolyticum; respiratory tract infections related to infection by Mycoplasma pneumoniae, Legionella pneumophila, Streptococcus pneumoniae, Haemophilus influenzae, or Chlamydia pneumoniae; uncomplicated skin and soft tissue infections, abscesses and osteomyelitis, and puerperal fever related to infection by Staphylococcus aureus, coagulase-positive staphylococci (i.e., S. epidermidis, S. hemolyticus, etc.), S. pyogenes, S. agalactiae, Streptococcal groups C-F (minute-colony streptococci), viridans streptococci, Corynebacterium spp., Clostridium spp., or Bartonella henselae; uncomplicated acute urinary tract infections related to infection by S. saprophyticus or Enterococcus spp.; urethritis and cervicitis; sexually transmitted diseases related to infection by Chlamydia trachomatis, Haemophilus ducreyi, Treponema pallidum, Ureaplasma urealyticum, or Nesseria gonorrheae; toxin diseases related to infection by S. aureus (food poisoning and Toxic shock syndrome), or Groups A, S, and C streptococci; ulcers related to infection by Helicobacter pylori; systemic febrile syndromes related to infection by Borrelia recurrentis; Lyme disease related to infection by Borrelia burgdorferi; conjunctivitis, keratitis, and dacrocystitis related to infection by C. trachomatis, N. gonorrhoeae, S. aureus, S. pneumoniae, S. pyogenes, H. influenzae, or Listeria spp.; disseminated Mycobacterium avium complex (MAC) disease related to infection by Mycobacterium avium, or Mycobacterium intracellulare; gastroenteritis related to infection by Campylobacter jejuni; odontogenic infection related to infection by viridans streptococci; persistent cough related to infection by Bordetella pertussis; gas gangrene related to infection by Clostridium perfringens or Bacteroides spp.; skin infection by S. aureus, Propionibacterium acne; atherosclerosis related to infection by Helicobacter pylori or Chlamydia pneumoniae; or the like.

17. A method of screening for a compound that modulates QS, biofilm formation, biofilm streamer formation, and/or a virulence factor production by a microorganism, wherein the method comprises contacting a compound with the surface of claim 1 and measuring whether QS, biofilm formation, biofilm streamer formation, and/or a virulence factor production by a microorganism is either increased, decreased or maintained.
